SEQUENCE LISTING (250 sequences)

SEQ ID NO: 1 - DNA, 1016 bp, Homo sapiens
  CDS (134)..(766)
agcgacctcagaggagtaaccgggccttaactttttgcgctcgttttgctataatttttctctatccacctccatcccacccccacaacactctttactgggggggtcttttgtgttccggatctccccctccatggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttgaagtgcgatagtgacattatggaagagtcaaaccacaaccattctttcttgtcatagttcccatcataaaataatgacccaaggagacgttcaaaatattaaagtctattttctactgagagactggatttggaaagaatattgagaaaaaaaaccaaaaaaaattttgactagaaatagatcatgatcactctttatatgtggattaagttcccttagatacattggattagtccttaccagtagac

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 358 bp, Homo sapiens
tcctttgacattgctagcaggttaactaatagaatggaaacttcagctatggggaaagatcctgggatattagaaccggagagcaccccatctttgtacagaaaactaagcctcagactgatgaaggcactttctagttacacagctagtgaggaagtcattaacaggagagaccctcccgatctagtatcttaacagacactgccttaacaatcattctcttgtttcttttaaccccttctcttcccaggcactgccggaggtattctgaaacacgtccgtctgtgttcccacccatatcttctttcgctttcccatttcctctttcctaaagtcgataccaagatacttgctttca

SEQ ID NO: 4 - DNA, 237 bp, Homo sapiens
gttgcacaatttccaaagaggagcttggctgaagaactaggcatgctcagtagccgggtggtcttcctcctcccccacccctccccccctttccttttcttttctcacccacatagaacttaggagctgagggaacctcagacaggtgagccctacaggtagcgaatgtgcccacggaaagttaatctgctacctcttcaggtgaacatttgcaagtctctaggtagacacgtaaat

SEQ ID NO: 5 - DNA, 52 bp, Homo sapiens
  variation (26)..(26): SNP rs1989754
agctcctctttggaaattgtgcaacgtgaaagcaagtatcttggtatcgact

SEQ ID NO: 6 - DNA, 248 bp, Homo sapiens
  variation (84)..(84): SNP rs1721100
aagtgcgatagtgacattatggaagagtcaaaccacaaccattctttcttgtcatagttcccatcataaaataatgacccaaggagacgttcaaaatattaaagtctattttctactgagagactggatttggaaagaatattgagaaaaaaaaccaaaaaaaattttgactagaaatagatcatgatcactctttatatgtggattaagttcccttagatacattggattagtccttaccagtagac

SEQ ID NO: 7 - DNA, 213 bp, Mus musculus
atgaatctagagccattgtttaaaaatcacagttcctgctgttaaataacaccgaagaagacgttcaggatattacgggagtctgcttttcactgaaagactctatttgggaagaaaattgagagtaaggaattaacttgaagcaaagcaagatcattctccgtaagtggattgtagttccttagacacgttgtttcagtcttaccagtagac

SEQ ID NO: 8 - DNA, 7770 bp, Homo sapiens
  CDS (1)..(7767)
atggaatcggaggaggagcagcacatgaccacgctgctgtgcatgggcttctcagaccccgccaccatccgcaaggccctgcgcctggccaagaacgacattaacgaggccgtggcactgctcaccaacgagcggccgggcctcgactacggcggctacgagcccatggacagcggcggcgggggcggcttcgaccccccgcccgcctaccacgaggtggtggacgcggagaagaatgatgagaatggaaactgctcaggggaaggaattgaattccctacaacaaatttatatgaactggaaagccgtgttttgactgatcattggtccatcccttacaagcgagaagaatcactaggcaaatgcctgttggcatctacctacctagcaagacttggtctttccgagtctgatgagaattgtagaaggtttatggacaggtgtatgcctgaagcatttaaaaagctcctgacatcaagtgctgttcacaagtggggtactgaaattcatgaaggaatttacaacatgttgatgctattaatagaactggtcgcagagagaataaaacgagatccaattcccattggtctcctgggtgtgcttacaatggctttcaatcctgataatgaataccattttaaaaacagaatgaaagtgtctcaaaggaattgggcacaagtgtctggagagggaactatgtttgctgtttcacctgtatcgactttccaaaaggagcctcatggatgggttgtggatttggtaaataagtttggagaattaggtggatttgcagcaatccaagccaagctccattcagaagatatagaacttggggctgtctcagcactgattcagcccttaggagtgtgtgcagagtacctcaattcctccgtggtacagcccatgctagacccagtcattcttactacaatccaggatgtacggagtgtagaagagaaagacctcaaagacaagagattggttagcatccctgagctcttgtctgccgttaagttactttgcatgcgcttccaaccggatctggtgacaattgtggatgaccttcgactagatattctattgcgcatgctgaaatcaccacatttcagtgctaagatgaattctctcaaagaagtaaccaaactaatagaagatagcactttatccaaatctgtgaagaatgctatagatacagacagattattagattggctagttgaaaactcagttctgtcgattgcactggaaggcaacatagaccaagcacaatactgtgaccgtataaagggaattattgaactcttgggtagtaaattgtcgttagatgaactcactaaaatttggaagatacagtcaggacaatcatctactgtgattgagaacattcatactattattgctgcagcggctgtgaaatttaattcagatcagcttaatcatttgtttgttctcattcagaagagctgggagactgagagtgatagagtaagacagaagcttttgagcctgattggacgaataggccgggaagctcgctttgagaccacttctggaaaggttttagacgtactctgggaactggctcaccttccaaccctgcccagtagccttattcagcaggccttggaggagcacctgacaatccttagtgatgcatatgcagtgaaagaagcaatcaagaggagctacatcatcaagtgcatagaagatattaagaggcctggagaatggtcaggtttggaaaaaaacaagaaggatggattcaagtcatctcagcttaataatccccagtttgtatgggtggtaccagctttgcgtcagctccatgaaattactcgctcattcataaaacaaacctatcaaaagcaagacaagagcattattcaagacttgaagaagaattttgaaatagtgaaattggtaacgggaagtttgatcgcttgtcatcggcttgcagctgctgtggccgggcctggaggcttaagtggctcgacactagtggatggccggtacacttaccgggagtatttagaggcacatctaaaatttctagcgtttttcttgcaagaagctactctgtatctgggctggaatcgtgccaaggagatctgggagtgtcttgkaactggccaggatgtttgtgaattagatagagagatgtgttttgaatggtttacaaaaggacagcatgatcttgagagtgatgttcagcagcagctcttcaaggaraaaattcttaaattggagtcatatgaaatcactatgaatggttttaacttatttaaaactttttttgaaaatgtgaatctttgtgatcatcgattgaaaagacaaggagctcagttgtatgtagaaaagctggaattgataggaatggatttcatttggaaaatagccatggaatcacctgatgaagaaattgctagtgaagctattcagctaatcataaactatagttacattaatctaaatcctagattaaagaaggattcagtatctttacataagaaattcattgctgattgctacacaagattagaagcagccagttcagcacttggtggccccactctaacacatgctgtgaccagagcaacaaaaatgcttacagcaactgccatgccaactgtagcaacctcagttcagtctccttatagatctactaaacttgtaataattgagagattgctgcttctggcagagcgctatgtgatcactatagaggatttttactctgttccacgaactattctacctcatggtgcctcatttcatggacatcttttaacccttaatgttacctatgagtctaccaaagataccttcactgtcgaggctcacagtaatgaaaccatagggagtgtccggtggaaactagccaagcagttgtgctctcctgtggataatatacagatatttacaaatgatagcctgctgacagtgaataaagatcaaaagctactccaccaactgggcttttctgatgaacaaatccttacagtgaagacttctggcagtgggaccccatctgggagttcagcagattcttcaaccagctccagcagcagcagcagtggggtttttagttcttcatatgccatggagcaggagaaatccctccctggtgtagtgatggctctcgtatgtaacgtatttgacatgctttatcagctcgccaatctggaagagccaaggataactctacgagtacggaagcttctgctcttgatacccactgatccagccattcaggaagcccttgatcaacttgattctttaggaagaaagaaaacattgctgtctgaatcaagttctcagtcctcaaaatctccatccctgtcatcaaagcaacagcaccagccaagtgccagttcaattttagaaagtctgtttcgatcttttgccccgggaatgtctaccttcagagtgctctacaacttagaagttctaagctccaaactcatgccaacagctgatgatgacatggccagaagctgtgccaaatccttctgtgaaaacttcctcaaagctggcggtttgagtttggttgtaaatgtcatgcagagagactccatcccatcagaagtagactatgaaacaaggcagggtgtttattccatctgtctacagcttgcaagatttttacttgtcggacaaacaatgtccacgttattagatgaagacctcaccaaagatggtatagaagcactttcttcccgcccattccgaaatgtcagccggcagacaagcagacagatgtccttatgtggtaccccagaaaagtcatcctaccgacagttgtccgtgtctgataggtcttctattagggttgaggaaatcatccctgctgctcgagttgcaatacaaacaatggaagtaagtgatttcacttctactgtggcttgcttcatgagattgtcatgggctgcggctgcaggacggcttgatcttgttgggagtagccagccaattaaagaaagtaattccctgtgtcctgctggaattcgaaacagactcagcagttcaggaagcaattgcagctctggaagtgaaggagaaccagtagccctgcatgcgggaatctgtgttcgacaacagtctgtatccaccaaagactcgctgattgcgggagaggctttgtctcttcttgttacgtgcctacagcttcggagccagcaactggcatctttctataacttgccctgtgttgctgatttcatcattgatattctgctcggatcaccaagtgctgagattcgccgggttgcctgtgatcagctgtacactcttagtcagacagacacatcagcgcatccagatgtgcagaagccaaatcagtttcttctaggcgtaatcctcacggctcagctgcctctctggtctccaactagtattatgagaggagtcaatcagagactgttatctcagtgtatggagtattttgatttgagatgccagttattagatgatctgacaacttcagaaatggagcagttaaggatcagcccagctacgatgcttgaagatgagattacttggctggataactttgaacctaatcgtacagctgaatgtgagaccagtgaagcggacaacatcttactggcagggcacttacgcctcatcaagacccttctttcactctgtggggcagaaaaggaaatgcttggttcatcactcattaaaccattgttagatgacttccttttccgagcttctagaattattttaaatagtcattctccagctggcagtgccgccatcagtcaacaggactttcatccaaagtgtagtacagcgaatagccgattggcagcctatgaagtccttgtgatgttggctgatagttcaccttcaaatcttcaaattattataaaagaactgctttctatgcatcaccagcctgaccctgctcttaccaaggagtttgattaccttcccccagtggatagcaggtccagttcagggtttgtggggctgagaaatggtggtgcaacttgttatatgaatgcagtcttccagcagctgtatatgcaacctgggctccctgagtcattactttcagtggatgatgacacagacaatccagatgatagcgtgttttaccaagtgcagtctctctttggacatttaatggaaagcaagctgcagtactatgtacctgagaatttttggaagattttcaagatgtggaataaagaactttatgtgagagaacagcaggatgcatatggattctttactagtctcattgatcagatggatgaatacctcaagaaaatggggagagaccaaatttttaagaatacatttcagggcatctactctgatcagaagatctgtaaagactgtcctcacagatatgagcgtgaagaagctttcatggctctcaatctaggagtgacttcttgtcagagtttggaaatttctttggaccaatttgttagaggagaagttctagaaggaagtaatgcgtactactgtgaaaagtgtaaagaaaagagaataacagtgaaaaggacctgtattaaatctttacctagcgtcttggtaattcacctaatgagatttgggtttgactgggaaagcggacgctccattaaatatgatgaacaaataaggtttccctggatgctaaacatggagccttacacagtttcaggaatggctcgccaagattcttcttctgaagttggggaaaatgggcgaagtgtggatcagggcggtggaggatccccacgaaaaaaggttgccctcacagaaaactatgaacttgtcggtgtcatcgtacacagtgggcaggcacacgcaggccactactattccttcattaaggacaggcgagggtgtggaaaaggaaagtggtataaatttaatgacacagttatagaagaatttgacctaaatgacgagaccctggagtatgaatgctttggaggagaatatagaccaaaagtttatgatcaaacaaacccatacactgatgtgcgccgaagatactggaatgcctatatgcttttctaccaaagggtgtctgatcagaactccccagtattaccaaagaaaagtcgagtcagcgttgtacggcaggaagctgaggatctctctctgtcagctccatcttcaccagaaatttcacctcagtcatcccctcggccccataggccgaacaatgaccggctgtctattcttaccaagctggttaaaaaaggcgagaagaaaggactgtttgtggagaaaatgcctgctcgaatataccagatggtgagagatgagaacctcaagtttatgaagaatagagatgtatacagtagtgattatttcagttttgttttgtctttagcttcattgaatgctactaaattaaagcatccatattatccttgcatggcaaaggtgagcttacagcttgctattcaattcctttttcaaacttatctacggacaaagaagaaactcagggttgatactgaagaatggattgctaccattgaagcattgctttcaaaaagttttgatgcttgtcagtggttagttgaatattttattagttctgaaggacgagaattgataaagattttcttactggagtgcaatgtgagagaagtacgagttgctgtggccaccattctggagaaaaccctagacagtgccttgttttatcaggataagttaaaaagccttcatcagttactggaggtactacttgctctgttggacaaagacgtcccagaaaattgtaaaaactgtgctcagtactttttcctgttcaacacttttgtacaaaagcaaggaattagggctggagatcttcttctgaggcattcagctctgcggcacatgatcagcttcctcctaggggccagtcggcaaaacaatcagatacgtcgatggagttcagcacaagcacgagaatttgggaatcttcacaatacagtggcgttacttgttttgcattcagatgtctcatcccaaaggaatgttgctcctggcatatttaagcaacgaccacccattagcattgctccctcaagccctctgttgcccctccatgaggaggtagaagccttgttgttcatgtctgaagggaaaccttacctgttagaggtaatgtttgctttgcgggagctgacaggctcgctcttggcactcattgagatggtagtgtactgctgtttctgtaatgagcatttttccttcacaatgctgcatttcattaagaaccaactagaaacggctccacctcatgagttaaagaatacgttccaactacttcatgaaatattggttattgaagatcctatacaagcagagcgagttaaatttgtgtttgagacagaaaatggattactagctttgatgcaccacagtaatcatgtggacagtagtcgctgctaccagtgtgtcaaatttcttgtcactcttgctcaaaagtgtcctgcagctaaggagtacttcaaggagaattcccaccactggagctgggctgtgcagtggctacagaagaagatgtcagaacattactggacaccacagagtaatgtctctaatgaaacatcaactggaaaaacctttcagcgaaccatttcagctcaggacacgttagcgtatgccacagctttgttgaatgaaaaagagcaatcaggaagcagtaatgggtcggagagtagtcctgccaatgagaacggagacaggcatctacagcagggttcagaatctcccatgatgattggtgagttgagaagtgaccttgatgatgttgatccctag

SEQ ID NO: 9 - protein, 2589 aa, Homo sapiens
  misc_feature (708)..(708): The ′Xaa′ at location 708 stands for Gly, or Val.
MESEEEQHMTTLLCMGFSDPATIRKALRLAKNDINEAVALLTNERPGLDYGGYEPMDSGGGGGFDPPPAYHEVVDAEKNDENGNCSGEGIEFPTTNLYELESRVLTDHWSIPYKREESLGKCLLASTYLARLGLSESDENCRRFMDRCMPEAFKKLLTSSAVHKWGTEIHEGIYNMLMLLIELVAERIKRDPIPIGLLGVLTMAFNPDNEYHFKNRMKVSQRNWAQVSGEGTMFAVSPVSTFQKEPHGWVVDLVNKFGELGGFAAIQAKLHSEDIELGAVSALIQPLGVCAEYLNSSVVQPMLDPVILTTIQDVRSVEEKDLKDKRLVSIPELLSAVKLLCMRFQPDLVTIVDDLRLDILLRMLKSPHFSAKMNSLKEVTKLIEDSTLSKSVKNAIDTDRLLDWLVENSVLSIALEGNIDQAQYCDRIKGIIELLGSKLSLDELTKIWKIQSGQSSTVIENIHTIIAAAAVKFNSDQLNHLFVLIQKSWETESDRVRQKLLSLIGRIGREARFETTSGKVLDVLWELAHLPTLPSSLIQQALEEHLTILSDAYAVKEAIKRSYIIKCIEDIKRPGEWSGLEKNKKDGFKSSQLNNPQFVWVVPALRQLHEITRSFIKQTYQKQDKSIIQDLKKNFEIVKLVTGSLIACHRLAAAVAGPGGLSGSTLVDGRYTYREYLEAHLKFLAFFLQEATLYLGWNRAKEIWECLXTGQDVCELDREMCFEWFTKGQHDLESDVQQQLFKEKILKLESYEITMNGFNLFKTFFENVNLCDHRLKRQGAQLYVEKLELIGMDFIWKIAMESPDEEIASEAIQLIINYSYINLNPRLKKDSVSLHKKFIADCYTRLEAASSALGGPTLTHAVTRATKMLTATAMPTVATSVQSPYRSTKLVIIERLLLLAERYVITIEDFYSVPRTILPHGASFHGHLLTLNVTYESTKDTFTVEAHSNETIGSVRWKLAKQLCSPVDNIQIFTNDSLLTVNKDQKLLHQLGFSDEQILTVKTSGSGTPSGSSADSSTSSSSSSSGVFSSSYAMEQEKSLPGVVMALVCNVFDMLYQLANLEEPRITLRVRKLLLLIPTDPAIQEALDQLDSLGRKKTLLSESSSQSSKSPSLSSKQQHQPSASSILESLFRSFAPGMSTFRVLYNLEVLSSKLMPTADDDMARSCAKSFCENFLKAGGLSLVVNVMQRDSIPSEVDYETRQGVYSICLQLARFLLVGQTMSTLLDEDLTKDGIEALSSRPFRNVSRQTSRQMSLCGTPEKSSYRQLSVSDRSSIRVEEIIPAARVAIQTMEVSDFTSTVACFMRLSWAAAAGRLDLVGSSQPIKESNSLCPAGIRNRLSSSGSNCSSGSEGEPVALHAGICVRQQSVSTKDSLIAGEALSLLVTCLQLRSQQLASFYNLPCVADFIIDILLGSPSAEIRRVACDQLYTLSQTDTSAHPDVQKPNQFLLGVILTAQLPLWSPTSIMRGVNQRLLSQCMEYFDLRCQLLDDLTTSEMEQLRISPATMLEDEITWLDNFEPNRTAECETSEADNILLAGHLRLIKTLLSLCGAEKEMLGSSLIKPLLDDFLFRASRIILNSHSPAGSAAISQQDFHPKCSTANSRLAAYEVLVMLADSSPSNLQIIIKELLSMHHQPDPALTKEFDYLPPVDSRSSSGFVGLRNGGATCYMNAVFQQLYMQPGLPESLLSVDDDTDNPDDSVFYQVQSLFGHLMESKLQYYVPENFWKIFKMWNKELYVREQQDAYGFFTSLIDQMDEYLKKMGRDQIFKNTFQGIYSDQKICKDCPHRYEREEAFMALNLGVTSCQSLEISLDQFVRGEVLEGSNAYYCEKCKEKRITVKRTCIKSLPSVLVIHLMRFGFDWESGRSIKYDEQIRFPWMLNMEPYTVSGMARQDSSSEVGENGRSVDQGGGGSPRKKVALTENYELVGVIVHSGQAHAGHYYSFIKDRRGCGKGKWYKFNDTVIEEFDLNDETLEYECFGGEYRPKVYDQTNPYTDVRRRYWNAYMLFYQRVSDQNSPVLPKKSRVSVVRQEAEDLSLSAPSSPEISPQSSPRPHRPNNDRLSILTKLVKKGEKKGLFVEKMPARIYQMVRDENLKFMKNRDVYSSDYFSFVLSLASLNATKLKHPYYPCMAKVSLQLAIQFLFQTYLRTKKKLRVDTEEWIATIEALLSKSFDACQWLVEYFISSEGRELIKIFLLECNVREVRVAVATILEKTLDSALFYQDKLKSLHQLLEVLLALLDKDVPENCKNCAQYFFLFNTFVQKQGIRAGDLLLRHSALRHMISFLLGASRQNNQIRRWSSAQAREFGNLHNTVALLVLHSDVSSQRNVAPGIFKQRPPISIAPSSPLLPLHEEVEALLFMSEGKPYLLEVMFALRELTGSLLALIEMVVYCCFCNEHFSFTMLHFIKNQLETAPPHELKNTFQLLHEILVIEDPIQAERVKFVFETENGLLALMHHSNHVDSSRCYQCVKFLVTLAQKCPAAKEYFKENSHHWSWAVQWLQKKMSEHYWTPQSNVSNETSTGKTFQRTISAQDTLAYATALLNEKEQSGSSNGSESSPANENGDRHLQQGSESPMMIGELRSDLDDVDP

SEQ ID NO: 10 - DNA, 60 bp, Homo sapiens
actcgggtggacctcagcagctcagtcctcacagcaggaaggactcaccaccgactggaa

SEQ ID NO: 11 - DNA, 100 bp, Homo sapiens
actcgggtggacctcagcagctcagtcctcccaggagactctgtggggctggctgtcattctgcacactgacagcaggaaggactcaccaccagctggaa

SEQ ID NO: 12 - DNA, 21 bp, Artificial
  Oligonucleotide primer
acatgtcacttttgcttccct

SEQ ID NO: 13 - DNA, 23 bp, Artificial
  Oligoncucleotide primer
aggccatgctccatgcagactgc

SEQ ID NO: 14 - DNA, 20 bp, Artificial
  Oligonucleotide primer
gggctgctttctggcatatg

SEQ ID NO: 15 - DNA, 20 bp, Artificial
  Oligonucleotide primer
cctcacttctgtcacaggtc

SEQ ID NO: 16 - DNA, 20 bp, Artificial
  Oligonucleotide probe
aggaaccacaggtgagggtg

SEQ ID NO: 17 - DNA, 23 bp, Artificial
  Oligonucleotide probe
agaaggaaccacaggtgagggta

SEQ ID NO: 18 - DNA, 24 bp, Artificial
  Oligonucleotide probe
agccccagagacccccaggcagtc

SEQ ID NO: 19 - DNA, 25 bp, Artificial
  Oligonucleotide primer
ccacccgggagcccaagaaggtgcc

SEQ ID NO: 20 - DNA, 22 bp, Artificial
  Oligonucleotide primer
ctggtgcttcaggttctcagtg

SEQ ID NO: 21 - DNA, 20 bp, Artificial
  Oligonucleotide probe
gggagcccaagaaggtggcg

SEQ ID NO: 22 - DNA, 23 bp, Artificial
  Oligonucleotide probe
cccgggagcccaagaaggtggca

SEQ ID NO: 23 - DNA, 35 bp, Artificial
  Oligonucleotide probe
gtggtccgtactccacccaagtcgccgtcttccgc

SEQ ID NO: 24 - DNA, 19 bp, Artificial
  Oligonucleotide primer
cgagtcctggcttcactcc

SEQ ID NO: 25 - DNA, 20 bp, Artificial
  Oligonucleotide primer
cttccaggcacagccatacc

SEQ ID NO: 26 - DNA, 20 bp, Artificial
  Oligonucleotide probe
ccatgccagacctgaagaac

SEQ ID NO: 27 - DNA, 23 bp, Artificial
  Oligonucleotide probe
tgcccatgccagacctgaagaat

SEQ ID NO: 28 - DNA, 28 bp, Artificial
  Oligonucleotide probe
gtcaagtccaagatcggctccactgaga

SEQ ID NO: 29 - DNA, 19 bp, Artificial
  Oligonculeotide primer
cgagtcctggcttcactcc

SEQ ID NO: 30 - DNA, 20 bp, Artificial
  Oligonucleotide primer
cttccaggcacagccatacc

SEQ ID NO: 31 - DNA, 20 bp, Artificial
  Oligonucleotide probe
agaacctgaagcaccagcca

SEQ ID NO: 32 - DNA, 23 bp, Artificial
  Oligonucleotide probe
ctgagaacctgaagcaccagccg

SEQ ID NO: 33 - DNA, 26 bp, Artificial
  Oligonucleotide probe
ggaggcgggaaggtgagagtggctgg

SEQ ID NO: 34 - DNA, 20 bp, Artificial
  Oligonucleotide primer
gctcattctctctcctcctc

SEQ ID NO: 35 - DNA, 25 bp, Artificial
  Oligonucleotide primer
ccaggactcctccaccccatgcagc

SEQ ID NO: 36 - DNA, 20 bp, Artificial
  Oligonucleotide probe
ggtgagggttgggacgggaa

SEQ ID NO: 37 - DNA, 23 bp, Artificial
  Oligonucleotide probe
gaaggtgagggttgggacgggag

SEQ ID NO: 38 - DNA, 37 bp, Artificial
  Oligonucleotide probe
ggtgcagggggtggaggagtcctggtgaggctggaac

SEQ ID NO: 39 - DNA, 18 bp, Artificial
  Oligonucleotide primer
ggtgggtcctctgtgcaa

SEQ ID NO: 40 - DNA, 27 bp, Artificial
  Oligoncucleotide primer
ggctgattattttaggaccaggaaaca

SEQ ID NO: 41 - DNA, 20 bp, Artificial
  Oligonucleotide probe
ctattgactcatatgccttg

SEQ ID NO: 42 - DNA, 19 bp, Artificial
  Oligonucleotide probe
tattgactcatacgccttg

SEQ ID NO: 43 - DNA, 22 bp, Artificial
  Oligonucleotide primer
tgcctgacccttactgcaattt

SEQ ID NO: 44 - DNA, 24 bp, Artificial
  Oligonucleotide primer
cctatgcacctacctacgtctctt

SEQ ID NO: 45 - DNA, 24 bp, Artificial
  Oligonucleotide probe
ttttaaaagctcataagctagaac

SEQ ID NO: 46 - DNA, 18 bp, Artificial
  Oligonucleotide probe
aagctcataggctagaac

SEQ ID NO: 47 - DNA, 18 bp, Artificial
  Oligonucleotide primer
gcctcccaggaacaggat

SEQ ID NO: 48 - DNA, 20 bp, Artificial
  Oligonucleotide primer
cgctgagaaggtgccatttt

SEQ ID NO: 49 - DNA, 20 bp, Artificial
  Oligoncucleotide probe
ccatagaattcacgggacaa

SEQ ID NO: 50 - DNA, 20 bp, Artificial
  Oligonucleotide probe
ccatagaattcatgggacaa

SEQ ID NO: 51 - DNA, 26 bp, Artificial
  Oligonucleotide primer
gtgcttgactgagttgattcttagtg

SEQ ID NO: 52 - DNA, 23 bp, Artificial
  Oligonucleotide primer
ggacagacaaccacagagttacg

SEQ ID NO: 53 - DNA, 17 bp, Artificial
  Oligonucleotide probe
acttctctccgtctgtc

SEQ ID NO: 54 - DNA, 17 bp, Artificial
  Oligonucleotide probe
acttctctccatctgtc

SEQ ID NO: 55 - DNA, 24 bp, Artificial
  Oligonucleotide primer
ttccttcaccctcatacaaacatc

SEQ ID NO: 56 - DNA, 21 bp, Artificial
  Oligonucleotide primer
gccaacgttcctgctgaatag

SEQ ID NO: 57 - DNA, 28 bp, Artificial
  Oligonucleotide probe
ctgctcttttgagaccattcgatcctct

SEQ ID NO: 58 - DNA, 24 bp, Artificial
  Oligonucleotide probe
tgctcttttgaggccattcgatcc

SEQ ID NO: 59 - DNA, 31 bp, Artificial
  Oligonucleotide primer
agtgtgactttattgaaaacatgatgctttt

SEQ ID NO: 60 - DNA, 32 bp, Artificial
  Oligonucleotide primer
gcaatcctttgttatattttacctctgagagt

SEQ ID NO: 61 - DNA, 16 bp, Artificial
  Oligonucleotide probe
ccctgtgttatttatg

SEQ ID NO: 62 - DNA, 16 bp, Artificial
  Oligonucleotide probe
ccctgtgttctttatg

SEQ ID NO: 63 - DNA, 19 bp, Artificial
  Oligonucleotide primer
caccatgcctggccaaaag

SEQ ID NO: 64 - DNA, 21 bp, Artificial
  Oligonucleotide primer
ccggttctcttccttcagagg

SEQ ID NO: 65 - DNA, 20 bp, Artificial
  Oligonucleotide probe
aaagcgtagttaaaagcata

SEQ ID NO: 66 - DNA, 19 bp, Artificial
  Oligoncucleotide probe
aagcgtagttaagagcata

SEQ ID NO: 67 - DNA, 21 bp, Artificial
  Oligonucleotide primer
gggaatcatggcaacgagtct

SEQ ID NO: 68 - DNA, 21 bp, Artificial
  Oligonucleotide primer
agtctgagatgcggtgaacac

SEQ ID NO: 69 - DNA, 16 bp, Artificial
  Oligonucleotide probe
aaagcttgggaggcag

SEQ ID NO: 70 - DNA, 14 bp, Artificial
  Oligonucleotide probe
agcttggaaggcag

SEQ ID NO: 71 - DNA, 24 bp, Artificial
  Oligonucleotide primer
ggcagaagtcacagctataactca

SEQ ID NO: 72 - DNA, 16 bp, Artificial
  Oligonucleotide primer
aggcggcgtggagatc

SEQ ID NO: 73 - DNA, 13 bp, Artificial
  Oligonucleotide probe
ctcccggcacgcc

SEQ ID NO: 74 - DNA, 13 bp, Artificial
  Oligonucleotide probe
ctccccgcacgcc

SEQ ID NO: 75 - DNA, 28 bp, Artificial
  Oligonucleotide primer
tcacagtttagagcagttaaacaaagga

SEQ ID NO: 76 - DNA, 25 bp, Artificial
  Oligonucleotide primer
aggcacaacattctgaagagtgatt

SEQ ID NO: 77 - DNA, 20 bp, Artificial
  Oligonucleotide probe
aagaatgatttgcataataa

SEQ ID NO: 78 - DNA, 19 bp, Artificial
  Oligonucleotide probe
agaatgatttgcgtaataa

SEQ ID NO: 79 - DNA, 30 bp, Artificial
  Oligonucleotide primer
tgatggactgccaataatatttttgtttcc

SEQ ID NO: 80 - DNA, 34 bp, Artificial
  Oligonucleotide primer
gcagaaaagagtacagtataataaataacaccca

SEQ ID NO: 81 - DNA, 18 bp, Artificial
  Oligonucleotide probe
cattttgtgttatttgcc

SEQ ID NO: 82 - DNA, 17 bp, Artificial
  Oligonucleotide probe
attttgtgttgtttgcc

SEQ ID NO: 83 - DNA, 29 bp, Artificial
  Oligonucleotide primer
ccaattttccatccatagatgcaaagatt

SEQ ID NO: 84 - DNA, 20 bp, Artificial
  Oligonucleotide primer
cttggcctcccaaagtgttg

SEQ ID NO: 85 - DNA, 13 bp, Artificial
  Oligonucleotide probe
ccccggccccctt

SEQ ID NO: 86 - DNA, 13 bp, Artificial
  Oligonucleotide probe
ccccagccccctt

SEQ ID NO: 87 - DNA, 22 bp, Artificial
  Oligonucleotide primer
tggataaaccttgcaaacatgc

SEQ ID NO: 88 - DNA, 23 bp, Artificial
  Oligonucleotide primer
gggaacagatcatgacttgccta

SEQ ID NO: 89 - DNA, 20 bp, Artificial
  Oligonucleotide probe
atatgatttgtatgaaatgt

SEQ ID NO: 90 - DNA, 20 bp, Artificial
  Oligonculeotide probe
tatgatttctatgaaatgtt

SEQ ID NO: 91 - DNA, 21 bp, Artificial
  Oligonucleotide primer
tttgtcagccaagcactggtt

SEQ ID NO: 92 - DNA, 26 bp, Artificial
  Oligonucleotide primer
gctcataaactcagtgaaggaatgaa

SEQ ID NO: 93 - DNA, 17 bp, Artificial
  Oligonucleotide probe
atctgggagtaagatag

SEQ ID NO: 94 - DNA, 19 bp, Artificial
  Oligonucleotide probe
atctggtagtaagatagac

SEQ ID NO: 95 - DNA, 23 bp, Artificial
  Oligonucleotide primer
ctgcctgctatctgtcatcttca

SEQ ID NO: 96 - DNA, 20 bp, Artificial
  Oligonucleotide primer
gtcctggccaaagcaatcag

SEQ ID NO: 97 - DNA, 18 bp, Artificial
  Oligonucleotide probe
caagaggaagacatagtt

SEQ ID NO: 98 - DNA, 16 bp, Artificial
  Oligonucleotide probe
agaggaaggcatagtt

SEQ ID NO: 99 - DNA, 17 bp, Artificial
  Oligonucleotide primer
ggcccctctccgtggat

SEQ ID NO: 100 - DNA, 25 bp, Artificial
  Oligonucleotide primer
ttaggcatttgcttcatttatctga

SEQ ID NO: 101 - DNA, 25 bp, Artificial
  Oligonucleotide probe
tctccctcctgctctcataccaccc

SEQ ID NO: 102 - DNA, 25 bp, Artificial
  Oligonucleotide probe
tctccctcctgctttcataccaccc

SEQ ID NO: 103 - DNA, 21 bp, Artificial
  Oligonucleotide primer
gtggcagaagtagcactgaga

SEQ ID NO: 104 - DNA, 25 bp, Artificial
  Oligonucleotide primer
gccacagagggaacttgtttttaac

SEQ ID NO: 105 - DNA, 18 bp, Artificial
  Oligonucleotide probe
cagagaaagtgacagatt

SEQ ID NO: 106 - DNA, 20 bp, Artificial
  Oligonucleotide probe
aacagagaaagtaacagatt

SEQ ID NO: 107 - DNA, 28 bp, Artificial
  Oligonucleotide primer
ccaatacagagcacttttacattcatta

SEQ ID NO: 108 - DNA, 26 bp, Artificial
  Oligonucleotide primer
aggtatgaaattgggtgtattgctaa

SEQ ID NO: 109 - DNA, 27 bp, Artificial
  Oligonucleotide probe
tggagtgaggcaaactaagtcccagaa

SEQ ID NO: 110 - DNA, 28 bp, Artificial
  Oligonucleotide probe
agtgaggcaaactgagtcccagaaactc

SEQ ID NO: 111 - DNA, 25 bp, Artificial
  Oligonucleotide primer
cacaaagaacactggcattttaaga

SEQ ID NO: 112 - DNA, 27 bp, Artificial
  Oligonucleotide primer
ttctcaaaatagctccacagtgtatgt

SEQ ID NO: 113 - DNA, 25 bp, Artificial
  Oligonucleotide probe
accaaacaaagcagaatgtcaggcc

SEQ ID NO: 114 - DNA, 27 bp, Artificial
  Oligonucleotide probe
ccaaacaaagtagaatgtcaggccctg

SEQ ID NO: 115 - DNA, 18 bp, Artificial
  Oligonucleotide primer
cggagctgcctgctagtc

SEQ ID NO: 116 - DNA, 18 bp, Artificial
  Oligonucleotide primer
gcccaagggctgaagagt

SEQ ID NO: 117 - DNA, 15 bp, Artificial
  Oligonucleotide probe
cagtgctaggtgccg

SEQ ID NO: 118 - DNA, 15 bp, Artificial
  Oligonucleotide probe
cagtgctaagtgccg

SEQ ID NO: 119 - DNA, 20 bp, Artificial
  Oligonucleotide primer
ccctgtttgcctggatgtca

SEQ ID NO: 120 - DNA, 20 bp, Artificial
  Oligonucleotide primer
ggagcaggcagcaatctttg

SEQ ID NO: 121 - DNA, 14 bp, Artificial
  Oligonucleotide probe
ctgttgcacaggct

SEQ ID NO: 122 - DNA, 14 bp, Artificial
  Oligonucleotide probe
ctgttgcgcaggct

SEQ ID NO: 123 - DNA, 25 bp, Artificial
  Oligonucleotide primer
accactctactgcaagtctcatgta

SEQ ID NO: 124 - DNA, 34 bp, Artificial
  Oligonucleotide primer
tcaccaaataaataatgcatattttcccaacaat

SEQ ID NO: 125 - DNA, 22 bp, Artificial
  Oligonucleotide probe
ctgatacaaccaattattcata

SEQ ID NO: 126 - DNA, 21 bp, Artificial
  Oligonucleotide probe
tgatacaaccaattgttcata

SEQ ID NO: 127 - DNA, 26 bp, Artificial
  Oligonucleotide primer
gtgtgttatccttggtcagactgatg

SEQ ID NO: 128 - DNA, 23 bp, Artificial
  Oligonucleotide primer
ctgtgtgaccagggatgttcatt

SEQ ID NO: 129 - DNA, 25 bp, Artificial
  Oligonucleotide probe
ccttctgcttgtccccccaggttct

SEQ ID NO: 130 - DNA, 24 bp, Artificial
  Oligonucleotide probe
ccttctgcttgttcccccaggttc

SEQ ID NO: 131 - DNA, 24 bp, Artificial
  Oligonucleotide primer
cacacacacacacacacacattat

SEQ ID NO: 132 - DNA, 30 bp, Artificial
  Oligonucleotide primer
ggctgggaaaaaatatttgcaaagtacata

SEQ ID NO: 133 - DNA, 20 bp, Artificial
  Oligonucleotide probe
tcgctctctctctctatata

SEQ ID NO: 134 - DNA, 19 bp, Artificial
  Oligonucleotide probe
cgctctctctctatatata

SEQ ID NO: 135 - DNA, 27 bp, Artificial
  Oligonucleotide primer
tctctgctgatttgtcatgtacagttt

SEQ ID NO: 136 - DNA, 26 bp, Artificial
  Oligonucleotide primer
gatgtggagaaacaactgttaaagca

SEQ ID NO: 137 - DNA, 19 bp, Artificial
  Oligonucleotide probe
atctggaaatcatatattg

SEQ ID NO: 138 - DNA, 18 bp, Artificial
  Oligonucleotide probe
tctggaaatcgtatattg

SEQ ID NO: 139 - DNA, 22 bp, Artificial
  Oligonucleotide primer
catcttctgggcataccacagt

SEQ ID NO: 140 - DNA, 35 bp, Artificial
  Oligonucleotide primer
tcttttggatttcatgtatttttaaagtgtgaaca

SEQ ID NO: 141 - DNA, 19 bp, Artificial
  Oligonucleotide probe
tttattgggtgcctacttt

SEQ ID NO: 142 - DNA, 14 bp, Artificial
  Oligonucleotide probe
tgggtgcctgcttt

SEQ ID NO: 143 - DNA, 25 bp, Artificial
  Oligonucleotide primer
tgtccatcacctaactgaacttcct

SEQ ID NO: 144 - DNA, 21 bp, Artificial
  Oligonucleotide primer
cactgtgtaccagggcaaaga

SEQ ID NO: 145 - DNA, 14 bp, Artificial
  Oligonucleotide probe
agggctcaacactg

SEQ ID NO: 146 - DNA, 15 bp, Artificial
  Oligonucleotide probe
aagggctcgacactg

SEQ ID NO: 147 - DNA, 29 bp, Artificial
  Oligonucleotide primer
gcttttccagtatgagagtagctttaaga

SEQ ID NO: 148 - DNA, 24 bp, Artificial
  Oligonucleotide primer
cgaactcctgacctcaagtgattc

SEQ ID NO: 149 - DNA, 16 bp, Artificial
  Oligonucleotide probe
agtggctcacacctgt

SEQ ID NO: 150 - DNA, 14 bp, Artificial
  Oligonucleotide probe
tggctcacgcctgt

SEQ ID NO: 151 - DNA, 25 bp, Artificial
  Oligonucleotide primer
agcagaaacttgtttaccactcact

SEQ ID NO: 152 - DNA, 24 bp, Artificial
  Oligonucleotide primer
agagaaagatagtgggccatacca

SEQ ID NO: 153 - DNA, 18 bp, Artificial
  Oligonucleotide probe
tcacctactcggtgtcag

SEQ ID NO: 154 - DNA, 20 bp, Artificial
  Oligonucleotide probe
tatcacctactctgtgtcag

SEQ ID NO: 155 - DNA, 22 bp, Artificial
  Oligonucleotide primer
cacatggcaaatggtgacacaa

SEQ ID NO: 156 - DNA, 28 bp, Artificial
  Oligonucleotide primer
gtaagcccagttttaaaaaatcccttca

SEQ ID NO: 157 - DNA, 18 bp, Artificial
  Oligonucleotide probe
ccttactttatcaggccc

SEQ ID NO: 158 - DNA, 17 bp, Artificial
  Oligonucleotide probe
cttactttttcaggccc

SEQ ID NO: 159 - DNA, 20 bp, Artificial
  Oligonucleotide primer
caaccatcgcaagcgttagc

SEQ ID NO: 160 - DNA, 18 bp, Artificial
  Oligonucleotide primer
ccccgcgaagggaagaag

SEQ ID NO: 161 - DNA, 15 bp, Artificial
  Oligonucleotide probe
tcaggaggccccgct

SEQ ID NO: 162 - DNA, 13 bp, Artificial
  Oligonucleotide probe
aggaggcgccgct

SEQ ID NO: 163 - DNA, 25 bp, Artificial
  Oligonucleotide primer
ccaaggacctccataaatagtgaca

SEQ ID NO: 164 - DNA, 21 bp, Artificial
  Oligonucleotide primer
acagaggtagggctgcaactg

SEQ ID NO: 165 - DNA, 28 bp, Artificial
  Oligonucleotide probe
catgactttgcaagagaccagaagcatt

SEQ ID NO: 166 - DNA, 26 bp, Artificial
  Oligonucleotide probe
atgactttgcaagaggccagaagcat

SEQ ID NO: 167 - DNA, 21 bp, Artificial
  Oligonucleotide primer
gtcttggcctgttctgcaaag

SEQ ID NO: 168 - DNA, 44 bp, Artificial
  Oligonucleotide primer
ggtgtgtcatatagtacattattacatgatttagaatctatttt

SEQ ID NO: 169 - DNA, 22 bp, Artificial
  Oligoncucleotide probe
ataatcactattgcttactttt

SEQ ID NO: 170 - DNA, 17 bp, Artificial
  Oligonucleotide probe
cactattgcctactttt

SEQ ID NO: 171 - DNA, 21 bp, Artificial
  Oligonucleotide primer
ggagcaagtcacctcttacgt

SEQ ID NO: 172 - DNA, 23 bp, Artificial
  Oligonucleotide primer
ttcctgcacaagctctctctttt

SEQ ID NO: 173 - DNA, 13 bp, Artificial
  Oligonucleotide probe
atggcggaaggca

SEQ ID NO: 174 - DNA, 13 bp, Artificial
  Oligonucleotide probe
atggcagaaggca

SEQ ID NO: 175 - DNA, 29 bp, Artificial
  Oligonucleotide primer
agcaacatgatctgaagcgtataatatac

SEQ ID NO: 176 - DNA, 24 bp, Artificial
  Oligonucleotide primer
gccacttctagtccccttatttcc

SEQ ID NO: 177 - DNA, 28 bp, Artificial
  Oligonucleotide probe
cgatcctgatgaagctttacagtgagga

SEQ ID NO: 178 - DNA, 28 bp, Artificial
  Oligonucleotide probe
cgatcctgatgaacctttacagtgagga

SEQ ID NO: 179 - DNA, 28 bp, Artificial
  Oligonucleotide primer
caataccaagggtttcagtaattatgtt

SEQ ID NO: 180 - DNA, 30 bp, Artificial
  Oligonucleotide primer
gcttggagacatattgaataaactgtagtc

SEQ ID NO: 181 - DNA, 29 bp, Artificial
  Oligonucleotide probe
agcaaacgattgcagatcacatgatttaa

SEQ ID NO: 182 - DNA, 26 bp, Artificial
  Oligonucleotide probe
agcaaacgattgcagaccacatgatt

SEQ ID NO: 183 - DNA, 26 bp, Artificial
  Oligonucleotide primer
ctccttactaacgtagagctcaccta

SEQ ID NO: 184 - DNA, 26 bp, Artificial
  Oligonucleotide primer
acacaagaaagaacatagtggatgct

SEQ ID NO: 185 - DNA, 20 bp, Artificial
  Oligonucleotide probe
aaaccctttttaagccttta

SEQ ID NO: 186 - DNA, 20 bp, Artificial
  Oligonucleotide probe
aaaccctttttaaaccttta

SEQ ID NO: 187 - DNA, 23 bp, Artificial
  Oligonucleotide primer
cgtgcctgtttgttgcttaaatg

SEQ ID NO: 188 - DNA, 27 bp, Artificial
  Oligonucleotide primer
agaccaagggataaacagttgaaaagt

SEQ ID NO: 189 - DNA, 24 bp, Artificial
  Oligonucleotide probe
tattctcacatatttatcattgtt

SEQ ID NO: 190 - DNA, 19 bp, Artificial
  Oligonucleotide probe
tcacatatttgtcattgtt

SEQ ID NO: 191 - DNA, 22 bp, Artificial
  Oligonucleotide primer
cccacctggagattctgactca

SEQ ID NO: 192 - DNA, 23 bp, Artificial
  Oligonucleotide primer
ctccctcccttcatcagttgttc

SEQ ID NO: 193 - DNA, 14 bp, Artificial
  Oligonucleotide probe
ccacccagacccag

SEQ ID NO: 194 - DNA, 14 bp, Artificial
  Oligonucleotide probe
ccacccacacccag

SEQ ID NO: 195 - DNA, 26 bp, Artificial
  Oligonucleotide primer
agaattcaatatggtgagatgaatgc

SEQ ID NO: 196 - DNA, 25 bp, Artificial
  Oligonucleotide primer
atcctctgaactgttctgagtgtca

SEQ ID NO: 197 - DNA, 22 bp, Artificial
  Oligonucleotide probe
tgccaaacccaagctgaaaggc

SEQ ID NO: 198 - DNA, 21 bp, Artificial
  Oligonucleotide probe
tgccaaacccacgctgaaagg

SEQ ID NO: 199 - DNA, 23 bp, Artificial
  Oligonucleotide primer
gtgctctgatagcaccagtgaga

SEQ ID NO: 200 - DNA, 27 bp, Artificial
  Oligonucleotide primer
gactggcaacttcttttaacattacct

SEQ ID NO: 201 - DNA, 27 bp, Artificial
  Oligonucleotide probe
aggcctaaaccctagaattggcaatga

SEQ ID NO: 202 - DNA, 23 bp, Artificial
  Oligonucleotide probe
aggcctaaaccctggaattggca

SEQ ID NO: 203 - DNA, 20 bp, Artificial
  Oligonucleotide primer
tgcccattacatgcctgaca

SEQ ID NO: 204 - DNA, 27 bp, Artificial
  Oligonucleotide primer
ccaggtaaacaaacaaatatgatatcg

SEQ ID NO: 205 - DNA, 27 bp, Artificial
  Oligonucleotide probe
tgtctcaagagttgagtggggaagaca

SEQ ID NO: 206 - DNA, 28 bp, Artificial
  Oligonucleotide probe
ctgtctcaagagttgattggggaagaca

SEQ ID NO: 207 - DNA, 25 bp, Artificial
  Oligonucleotide primer
gccagaaatcctactctttgggaaa

SEQ ID NO: 208 - DNA, 24 bp, Artificial
  Oligonucleotide primer
agcagaagtttggatggaggaaaa

SEQ ID NO: 209 - DNA, 16 bp, Artificial
  Oligonucleotide probe
caaatgctgcaagtac

SEQ ID NO: 210 - DNA, 16 bp, Artificial
  Oligonucleotide probe
caaatgctggaagtac

SEQ ID NO: 211 - DNA, 21 bp, Artificial
  Oligonucleotide primer
ctgggaccgaaaggagttagc

SEQ ID NO: 212 - DNA, 25 bp, Artificial
  Oligonucleotide primer
cagtttgctgggtactcactgataa

SEQ ID NO: 213 - DNA, 19 bp, Artificial
  Oligonucleotide probe
acatgattggatagagtta

SEQ ID NO: 214 - DNA, 19 bp, Artificial
  Oligonucleotide probe
acatgattggttagagtta

SEQ ID NO: 215 - DNA, 28 bp, Artificial
  Oligonucleotide primer
agtcccagttgaaacttactagatcaga

SEQ ID NO: 216 - DNA, 25 bp, Artificial
  Oligonucleotide primer
cagctattttactgtgcacaaccat

SEQ ID NO: 217 - DNA, 21 bp, Artificial
  Oligonucleotide probe
ataaatggtctctatggttct

SEQ ID NO: 218 - DNA, 16 bp, Artificial
  Oligonucleotide probe
tggtctctagggttct

SEQ ID NO: 219 - DNA, 27 bp, Artificial
  Oligonucleotide primer
aggcaaacaactttctcagtatcttct

SEQ ID NO: 220 - DNA, 30 bp, Artificial
  Oligonucleotide primer
acagttgcttctctttatgaaaatgatcct

SEQ ID NO: 221 - DNA, 16 bp, Artificial
  Oligonucleotide probe
agcacaaagagagaaa

SEQ ID NO: 222 - DNA, 17 bp, Artificial
  Oligonucleotide probe
cagcacaaatagagaaa

SEQ ID NO: 223 - DNA, 25 bp, Artificial
  Oligonucleotide primer
ggacactagaacctttgctacatct

SEQ ID NO: 224 - DNA, 26 bp, Artificial
  Oligonucleotide primer
ctgctgtttttgctagtatgcgtaat

SEQ ID NO: 225 - DNA, 17 bp, Artificial
  Oligonucleotide probe
ctgcaatttattttttg

SEQ ID NO: 226 - DNA, 17 bp, Artificial
  Oligonucleotide probe
ctgcaatttatattttg

SEQ ID NO: 227 - DNA, 27 bp, Artificial
  Oligonucleotide primer
gaccatgaaatacagagatgagtcaca

SEQ ID NO: 228 - DNA, 24 bp, Artificial
  Oligonucleotide primer
cctctgattggtcagtccttctca

SEQ ID NO: 229 - DNA, 15 bp, Artificial
  Oligonucleotide probe
ctcagggagattaca

SEQ ID NO: 230 - DNA, 16 bp, Artificial
  Oligonucleotide probe
tctcagggatattaca

SEQ ID NO: 231 - DNA, 25 bp, Artificial
  Oligonucleotide primer
ggatttcttcttggactcacactct

SEQ ID NO: 232 - DNA, 20 bp, Artificial
  Oligonucleotide primer
cccaacctgctcccactttt

SEQ ID NO: 233 - DNA, 18 bp, Artificial
  Oligonucleotide probe
cagtgaatttgcatttag

SEQ ID NO: 234 - DNA, 18 bp, Artificial
  Oligonucleotide probe
cagtgaatttgcgtttag

SEQ ID NO: 235 - DNA, 26 bp, Artificial
  Oligonucleotide primer
cctggaaaatctaatcgcatgaggta

SEQ ID NO: 236 - DNA, 23 bp, Artificial
  Oligonucleotide primer
ctgcccatgctgaaaatcctatg

SEQ ID NO: 237 - DNA, 18 bp, Artificial
  Oligonucleotide probe
ctggaaggaaaaccccat

SEQ ID NO: 238 - DNA, 17 bp, Artificial
  Oligonucleotide probe
tggaaggaaaacaccat

SEQ ID NO: 239 - DNA, 23 bp, Artificial
  Oligonucleotide primer
gcatgaggcactgagactaagtc

SEQ ID NO: 240 - DNA, 25 bp, Artificial
  Oligonucleotide primer
agtgcagtggaaatcagtctaaagg

SEQ ID NO: 241 - DNA, 14 bp, Artificial
  Oligonucleotide probe
tgccgccttttcat

SEQ ID NO: 242 - DNA, 15 bp, Artificial
  Oligonucleotide probe
ttgcccccttttcat

SEQ ID NO: 243 - DNA, 24 bp, Artificial
  Oligonucleotide primer
tcttttcagagctctcctcagact

SEQ ID NO: 244 - DNA, 23 bp, Artificial
  Oligonucleotide primer
gactgggaaggaacagagaaagg

SEQ ID NO: 245 - DNA, 17 bp, Artificial
  Oligonucleotide probe
actcattgacctcctcc

SEQ ID NO: 246 - DNA, 16 bp, Artificial
  Oligonucleotide probe
ctcattgaactcctcc

SEQ ID NO: 247 - DNA, 24 bp, Artificial
  Oligonucleotide primer
ctttccatttccctccactacact

SEQ ID NO: 248 - DNA, 30 bp, Artificial
  Oligonucleotide primer
aactacatagagactttcaaggtgaagaag

SEQ ID NO: 249 - DNA, 26 bp, Artificial
  Oligonucleotide probe
acttgtaagtctccgaccatgccatg

SEQ ID NO: 250 - DNA, 28 bp, Artificial
  Oligonucleotide probe
acttgtaagtctctgaccatgccatgct

(19) United States
(12) Patent Application Publication
(10) Pub. No.: US 2006/0068428 A1
(43) Pub. Date: Mar. 30, 2006

(54) Identification of genetic markers associated with parkinson disease

(75) Inventors: Jeffery M. Vance, Chapel Hill, NC (US); Yi-Ju Li, Durham, NC (US); Margaret A. Pericak-Vance, Chapel Hill, NC (US); Eden R. Martin, Hillsborough, NC (US); William K. Scott, Durham, NC (US); Michael A. Hauser, Durham, NC (US); Jeffrey M. Stajich, Hillsborough, NC (US); Sofia Oliveira, Lisbon (PT); Joelle van der Walt, Raleigh, NC (US)

Correspondence Address: MYERS BIGEL SIBLEY & SAJOVEC, PO BOX 37428, RALEIGH, NC, 27627, US

(73) Assignee: Duke University

(21) Appl. No.: 11/216,660

(22) Filed: Aug. 31, 2005

Related U.S. Application Data
(63) Continuation-in-part of application No. 10/979,297, filed on Nov. 2, 2004.
(60) Provisional application No. 60/516,861, filed on Nov. 3, 2003.

(51) Int. Cl. C12Q 1/68

(52) U.S. Cl. 435/6

(57) ABSTRACT

The present invention provides methods and compositions for screening a subject for Parkinson disease, for increased risk of developing Parkinson disease and/or for an earlier or later age of developing Parkinson disease, comprising detecting the presence of a genetic marker associated with Parkinson disease.

### CROSS REFERENCE TO RELATED APPLICATIONS

This application is a continuation-in-part of and claims priority to U.S. application Ser. No. 10/979,297, filed Nov. 2, 2004, which claims the benefit of U.S. Provisional Application Ser. No. 60/516,861, filed Nov. 3, 2003, the disclosures of each of which are incorporated herein by reference in their entireties.

### STATEMENT OF GOVERNMENT SUPPORT

This invention was made with Government support under grant numbers NS39764 and NS26630 from the National Institutes of Health and grant numbers R01 NS311530 and P50-NS-039764 from the National Institutes of Health/National Institute for Neurological Disorders and Stroke. The United States Government has certain rights in this invention.

### FIELD OF THE INVENTION

The present invention is directed to compositions and methods of screening a subject for Parkinson disease (PD), or increased risk of developing PD by identifying genetic markers associated with PD in the subject.

### BACKGROUND OF THE INVENTION

Parkinson disease is a progressive degenerative disease of the central nervous system. The risk of developing Parkinson disease increases with age, and afflicted individuals are usually adults over 40. Parkinson disease occurs in all parts of the world, and affects more than one million individuals in the United States alone.

While the primary cause of Parkinson disease is not known, it is characterized by degeneration of dopaminergic neurons of the substantia nigra. The substantia nigra is a portion of the lower brain, or brain stem, that helps control voluntary movements. The shortage of dopamine in the brain caused by the loss of these neurons is believed to cause the observable disease symptoms.

The symptoms of PD vary from patient to patient. The most common symptom is a paucity of movement: That is, rigidity characterized by an increased stiffness of voluntary skeletal muscles. Additional symptoms include resting tremor, bradykinesia (slowness of movement), poor balance, and walking problems. Common secondary symptoms include depression, sleep disturbance, dizziness, stooped posture, dementia, and problems with speech, breathing, and swallowing. The symptoms become progressively worse and ultimately result in death.

Surgical treatments available for PD include pallidotomy, brain tissue transplants, and deep brain stimulation. Such treatments are obviously highly invasive procedures accompanied by the usual risks of brain surgery, including stroke, partial vision loss, speech and swallowing difficulties, and confusion.

A variety of chemotherapeutic treatments for PD are also available. Perhaps the best known is administration of levodopa, a dopamine precursor. While levodopa administration can result in a dramatic improvement in symptoms, patients can experience serious side-effects, including nausea and vomiting. Concurrent carbidopa administration with levodopa is a significant improvement, with the addition of carbidopa inhibiting levodopa metabolism in the gut, liver and other tissues, thereby allowing more levodopa to reach the brain.

Amantadine hydrochloride is an indirect dopamine agonist (e.g., it either blocks dopamine reuptake or increases dopamine release), and is administered to patients as a monotherapy in the early stages of PD or administered in combination with levodopa (preferably also with carbidopa) as the disease progresses.

Anticholinergic agents such as trihexylphenidyl, benzotropine mesylate, and procyclidine can be administered to PD patients to decrease the activity of cholinergic systems of the brain in a substantially equivalent amount to the decrease experienced by the dopaminergic systems. The restore of a balance of activity between these two competing systems helps alleviate PD symptoms.

Selegiline or deprenyl administration to PD patients delays the need for levodopa administration when prescribed in the earliest stages of PD, and can also be used to boost the effectiveness of levodopa when administered in later stages of the disease.

Dopamine agonists such as bromocriptine, pergolide, pramipexole, and andropinirole are available for treating Parkinson disease, and can be administered to PD patients either alone or in combination with levodopa.

Catechol-O-methyltransferase (COMT) inhibitors such as tolcapone and entacapone can be administered to PD patients to inhibit COMT, an enzyme which breaks down levodopa before it reaches the brain. Obviously, COMT inhibitors must be used in combination with levodopa administration.

It will be appreciated that PD is unusual among neurodegenerative diseases in that a variety of treatments are available, including treatments that are beneficial in alleviating symptoms at even an early stage of the disease. Accordingly, means for screening subjects for Parkinson disease would extremely useful in insuring that appropriate treatments are promptly provided.

Genetic studies of common complex neurodegenerative diseases, such as Alzheimer's disease and Parkinson disease have focused on the identification of risk genes as targets for development of new treatments and improved diagnoses. This approach has identified the amyloid precursor protein (APP) (Goate et al., Nature 349:704-706 (1991)), presenilin 1 (PS1) (Sherrington et al., Nature 375:754-760 (1995)), presenilin 2 (PS2) (Levy-Lahad et al., Science 269:973-977 (1995); Rogaev et al., Nature 376:775-778 (1995)), and apolipoprotein E (APOE) (Corder et al., Science 261:921-923 (1993)) genes as contributing to risk in Alzheimer's disease. Three genes have been identified to associate with risk in Parkinson disease: alpha-synuclein (Polymeropoulos et al., Science 274:1197-1199 (1996)) for rare autosomal dominant early-onset Parkinson disease, Parkin (Abbas et al., Hum Mol Genet 8:567-574 (1999)) for rare autosomal recessive juvenile parkinsonism and autosomal recessive early-onset Parkinson disease, and tau (Martin et al., JAMA 286:2245-2250 (2001)) for classic Parkinson disease. Genomic screens in both Parkinson disease (Destefano et al., Neurology 57:1124-1126 (2001); Scott et al., JAMA 286:2239-2244 (2001)) and Alzheimer's disease (Kehoe et al., Hum Mol Genet 8:237-245 (1999); Pericak-Vance et al., Exp Gerontol 35:1343-1352 (2000)) have recently localized additional but, as yet, unknown risk genes.

Identification of further genes associated with PD provides new avenues of research with the potential to delay onset beyond the natural life span. Present knowledge about genes contributing to AAO in neurodegenerative diseases clearly lags behind the understanding of genes contributing to risk. There has been growing interest in using AAO information as a quantitative trait, to identify genes that influence onset of disease (Daw et al., Am J Hum Genet 64:839-851 (1999), Daw et al., Am J Hum Genet 66:196-204 (2000); Duggirala et al. Am J Hum Genet 64:1127-1140 (1999)). Rapid development of methods of mapping quantitative trait loci (QTLs) for general pedigrees (Goldgar, Am J Hum Genet 47:957-967 (1990); Amos, Am J Hum Genet 54:535-543 (1994); Blangero et al. Genet Epidemiol 14:959-964 (1997)) has now made the search for novel genes affecting AAO feasible. Thus, there is a continued need to develop new genetic linkages and markers as well as identifying new functional polymorphisms that are associated with Parkinson disease.

### SUMMARY OF THE INVENTION

The present invention provides a method of identifying a subject as having Parkinson disease or having an increased risk of developing Parkinson disease, comprising detecting in the subject the presence of a single nucleotide polymorphism in the human immunodeficiency virus type 1 enhancer binding protein 3 (HIVEP3) gene, wherein the single nucleotide polymorphism is correlated with Parkinson disease or an increased risk of developing Parkinson disease, thereby identifying the subject as having Parkinson disease or having an increased risk of developing Parkinson disease.

Additionally provided herein is a method of identifying a subject as having Parkinson disease or having an increased risk of developing Parkinson disease, comprising detecting in the subject the presence of a haplotype in the HIVEP3 gene of the subject comprising the following single nucleotide polymorphisms: rs648178_A (SNP 13_A), rs2038978_G (SNP 15_G), rs1039997_T (SNP 17_T), rs661225_G (SNP 19_G), and rs7554964_C (SNP 21_C).

The present invention further provides a method of identifying a subject as having Parkinson disease and/or having an earlier or later age of developing Parkinson disease and/or having an increased risk of developing Parkinson disease, comprising detecting in the subject the presence of a single nucleotide polymorphism in the eukaryotic translation initiation factor EIF2B3 gene, wherein the single nucleotide polymorphism is correlated with Parkinson disease and/or an earlier or later age of developing Parkinson disease and/or an increased risk of developing Parkinson disease, thereby identifying the subject as having Parkinson disease and/or having an earlier or later age of developing Parkinson disease and/or having an increased risk of developing Parkinson disease.

Furthermore, the present invention provides a method of identifying a subject as having Parkinson disease and/or having an increased risk of developing Parkinson disease and/or having an earlier or later age of developing Parkinson disease, comprising detecting in the subject the presence of a haplotype in the EIF2B3 gene of the subject comprising the following single nucleotide polymorphisms: rs263977_C (SNP 59_C), rs263978_C (SNP 60_C), rs546354_G (SNP 64_G), rs566063_T (SNP 65_T), and rs364482_G (SNP 66_G).

Also provided is a method of identifying a subject as having Parkinson disease and/or having an increased risk of developing Parkinson disease and/or having an earlier or later age of developing Parkinson disease, comprising detecting in the subject the presence of a haplotype in the EIF2B3 gene of the subject comprising the following single nucleotide polymorphisms: rs263977_A (SNP 59_A), rs263978_C (SNP 60_C), rs546354_A (SNP 64_A), rs566063_T (SNP 65_T), and rs364482_G (SNP 66_G).

In other embodiments, the present invention provides a method of identifying a subject as having Parkinson disease and/or having an increased risk of developing Parkinson disease and/or having an earlier or later age of developing Parkinson disease, comprising detecting in the subject the presence of a single nucleotide polymorphism in the ubiquitin-specific protease 24 (USP24) gene, wherein the single nucleotide polymorphism is correlated with Parkinson disease and/or an increased risk of developing Parkinson disease and/or an earlier or later age of developing Parkinson disease, thereby identifying the subject as having Parkinson disease and/or having an increased risk of developing Parkinson disease and/or having an earlier or later age of developing Parkinson disease.

Additionally provided is a method of identifying a subject as having Parkinson disease and/or having an increased risk of developing Parkinson disease and/or having an earlier or later age of developing Parkinson disease, comprising detecting in the subject the presence of a haplotype in the USP24 gene of the subject comprising the following single nucleotide polymorphisms: rs13312_C (SNP 218_C), rs1043671_T (SNP 219_T), and rs1165226_T (SNP 227_T).

Also provided herein is a method of identifying a subject as having Parkinson disease and/or having an increased risk of developing Parkinson disease and/or having an earlier or later age of developing Parkinson disease, comprising detecting in the subject the presence of a haplotype in the USP24 gene of the subject comprising the following single nucleotide polymorphisms: rs13312_C (SNP 218_C), rs1043671_T (SNP 219_T), and rs1165226_C (SNP 227_C).

The present invention additionally provides a method of identifying a subject as having Parkinson disease or having an increased risk of developing Parkinson disease, comprising detecting in the subject the presence of a single nucleotide polymorphism in the fibroblast growth factor 20 (FGF20) gene, wherein the single nucleotide polymorphism is correlated with Parkinson disease or an increased risk of developing Parkinson disease, thereby identifying the subject as having Parkinson disease or having an increased risk of developing Parkinson disease.

The present invention also provides a method of identifying a subject as having Parkinson disease or having an increased risk of developing Parkinson disease, comprising detecting in the subject the presence of a haplotype in the FGF20 gene of the subject comprising the following single nucleotide polymorphisms: 8p0217_A, rs1989756_G, rs1989754_C, rs1721100_C, and 8p0215_T.

A method is also provided herein of identifying a subject as having a decreased risk of developing Parkinson disease, comprising detecting in the subject the presence of a haplotype in the FGF20 gene of the subject comprising the following single nucleotide polymorphisms: 8p0217_A, rs1989756_G, rs1989754_G, rs1721100_G, and 8p0215_C.

In further embodiments, the present invention provides a method of identifying a subject as having Parkinson disease or having an increased risk of developing Parkinson disease, comprising detecting in the subject two or more genetic markers selected from the group consisting of: a) a single nucleotide polymorphism in the HIVEP3 gene, selected from the group consisting of rs648178 (SNP 13), rs661225 (SNP 19) and a combination of rs648178 (SNP 13) and rs661225 (SNP 19); b) a single nucleotide polymorphism in the EIF2B3 gene, selected from the group consisting of rs263977 (SNP 59), rs263978 (SNP 60), rs263965 (SNP 61), rs1022814 (SNP 62), rs12405721 (SNP 63), rs546354 (SNP 64), rs489676 (SNP 67 and any combination of rs263977 (SNP 59), rs263978 (SNP 60), rs263965 (SNP 61), rs1022814 (SNP 62), rs12405721 (SNP 63), rs546354 (SNP 64) and rs489676 (SNP 67); c) a single nucleotide polymorphism in the USP24 gene, selected from the group consisting of rs487230 (SNP 220), rs683880 (SNP 221), rs667353 (SNP 222), rs594226 (SNP 224), rs 1165226 (SNP 227), rs287235 (SNP 230), rs2047422 (SNP 231) and any combination of rs487230 (SNP 220), rs683880 (SNP 221), rs667353 (SNP 222), rs594226 (SNP 224), rs1165226 (SNP 227), rs287235 (SNP 230) and rs2047422 (SNP 231); d) a single nucleotide polymorphism in the FGF20 gene, selected from the group consisting of rs1989754, rs1721100, ss20399075, rs6985432, rs11203822, rs108881225, rs1227702208, rs172210282 and any combination of rs1989754, rs1721100, ss20399075, rs6985432, rs11203822, rs108881225, rs1227702208 and rs172210282; e) a functional polymorphism in the tau gene, selected from the group consisting of IVS3+9A→G, c1632A→G, c1716T→C, c1761G→A, IVS11+34G→A and any combination of IVS3+9A→G, c1632A→G, c1716T→C, c1761G→A and IVS11+34G→A; f) a deletion within base pairs 438-477 in exon 3 of the Parkin gene; g) a functional polymorphism in a segment of a chromosome selected from the group consisting of: a3) a segment of chromosome 2 bordered by D2S2982 and D2S1240; b3) a segment of chromosome 2 bordered by D2S1400 and D2S2291; c3) a segment of chromosome 2 bordered by D2S2161 and D2S1334; d3) a segment of chromosome 2 bordered by D2S161 and D2S2297; e3) a segment of chromosome 3 bordered by D3S1554 and D3S3631; f3) a segment of chromosome 3 bordered by D2S1251 and D3S3546; g3) a segment of chromosome 5 bordered by D5S2064 and D5S1968; h3) a segment of chromosome 5 bordered by D5S2027 and D5S1499; i3) a segment of chromosome 5 bordered by D5S816 and D5S1960; j3) a segment of chromosome 6 bordered by D6S1703 and D6S1027; k3) a segment of chromosome 6 bordered by D6S1581 and D6S2522; l3) a segment of chromosome 8 bordered by D8S504 and D8S258; m3) a segment of chromosome 9 bordered by D9S259 and D9S776; n3) a segment of chromosome 9 bordered by D9S1811 and D9S2168; o3) a segment of chromosome 10 bordered by D10S1122 and D10S1755; p3) a segment of chromosome 11 bordered by D11S4132 and D11S4112; q3) a segment of chromosome 12 bordered by D12S1042 and D12S64; r3) a segment of chromosome 14 bordered by D14S291 and D14S544; s3) a segment of chromosome 17 bordered by D17S1854 and D17S1293; t3) a segment of chromosome 17 bordered by D17S921 and D17S669; u3) a segment of chromosome 21 bordered by D21S1911 and D21S1895; v3) a segment of chromosome 22 bordered by D22S425 and D22S928; w3) a segment of chromosome X bordered by DXS6797 and DXS1205; and x3) a segment of chromosome X bordered by DXS9908 and X telomere; and any combination of (a3)-(x3), wherein the functional polymorphism is correlated with Parkinson disease or an increased risk of developing Parkinson disease; and h) any combination of (a)-(g) above, thereby identifying the subject as having Parkinson disease or having an increased risk of developing Parkinson disease.

The foregoing and other objects and aspects of the present invention are explained in detail in the drawings herein and the specification set forth below.

### BRIEF DESCRIPTION OF THE DRAWINGS

FIG. 1 demonstrates the alignment of human (SEQ ID NO:6) and mouse (SEQ ID NO:7) FGF20 3′UTR for rs1721100 and 8p0215.

FIG. 2 shows the mRNA (SEQ ID NO:8) and predicted protein sequence (SEQ ID NO:9) of the USP24_{L} gene. Protein sequence in bold corresponds to overlap with the AK127075 gene, and the underlined sequence matches the USP24 protein sequence. The DNA sequence in bold and underlined corresponds to the two additional exons of USP24_{L} in comparison to XM_{—}371254.

FIG. 3 shows the regions surrounding the 40 base deletion in Parkin Exon 3 (SEQ ID NOS:10 and 11).

### DETAILED DESCRIPTION OF THE EMBODIMENTS

The present invention is based on the identification of various genetic markers (e.g., single nucleotide polymorphisms or SNPs) associated with Parkinson disease and their use in methods of identifying a subject having Parkinson disease, as well as identifying a person having an increased risk of developing Parkinson disease and/or having an earlier or later age of developing Parkinson disease. Thus, in one embodiment, the present invention provides a method of identifying a subject as having Parkinson disease and/or having an increased risk of developing Parkinson disease, comprising detecting in the subject the presence of a single nucleotide polymorphism in the human immunodeficiency virus type 1 enhancer binding protein 3 (HIVEP3) gene, wherein the single nucleotide polymorphism is correlated with Parkinson disease and/or an increased risk of developing Parkinson disease, thereby identifying the subject as having Parkinson disease and/or having an increased risk of developing Parkinson disease. In this embodiment, the single nucleotide polypmorphism in the HIVEP2 gene can be, but is not limited to rs648178 (SNP 13), rs661225 (SNP 19) and/or a combination of rs648178 (SNP 13) and rs661225 (SNP 19).

Further provided herein is a method of identifying a subject as having Parkinson disease and/or having an increased risk of developing Parkinson disease, comprising detecting in the subject the presence of a haplotype in the HIVEP3 gene of the subject comprising the following single nucleotide polymorphisms: rs648178_A (SNP 13_A), rs2038978_G (SNP 15_G), rs1039997_T (SNP 17_T), rs661225_G (SNP 19_G), and rs7554964_C (SNP 21_C).

Identifying single nucleotide polymorphisms in the HIVEP3 gene and correlating them with Parkinson disease and/or an increased risk of developing Parkinson disease can be done according to the protocols set forth in the EXAMPLES section herein and according to well known art methods.

In other embodiments, the present invention provides a method of identifying a subject as having Parkinson disease and/or as having an earlier or later age of developing Parkinson disease and/or as having an increased risk of developing Parkinson disease, comprising detecting in the subject the presence of a single nucleotide polymorphism in the eukaryotic translation initiation factor EIF2B3 gene, wherein the single nucleotide polymorphism is correlated with Parkinson disease and/or an earlier or later age of developing Parkinson disease and/or an increased risk of developing Parkinson disease, thereby identifying the subject as having Parkinson disease and/or having an earlier or later age of developing Parkinson disease and/or having an increased risk of developing Parkinson disease. In this embodiment, the single nucleotide polymorphism in the EIF2B3 gene can be rs263977 (SNP 59), rs263978 (SNP 60), rs263965 (SNP 61), rs1022814 (SNP 62), rs12405721 (SNP 63), rs546354 (SNP 64), rs489676 (SNP 67) and/or any combination of rs263977 (SNP 59), rs263978 (SNP 60), rs263965 (SNP 61), rs1022814 (SNP 62), rs12405721 (SNP 63), rs546354 (SNP 64) and rs489676 (SNP 67).

The present invention additionally provides a method of identifying a subject as having Parkinson disease and/or having an increased risk of developing Parkinson disease and/or having an earlier or later age of developing Parkinson disease, comprising detecting in the subject the presence of a haplotype in the EIF2B3 gene of the subject comprising the following single nucleotide polymorphisms: rs263977_C (SNP 59_C), rs263978_C (SNP 60_C), rs546354_G (SNP 64_G), rs566063_T (SNP 65_T), and rs364482_G (SNP 66_G), or a haplotype in the EIF2B3 gene of the subject comprising the following single nucleotide polymorphisms: rs263977_A (SNP 59_A), rs263978_C (SNP 60_C), rs546354_A (SNP 64_A), rs566063_T (SNP 65_T), and rs364482_G (SNP 66_G).

Identifying single nucleotide polymorphisms in the EIF2B3 gene and correlating them with Parkinson disease and/or an increase risk of developing Parkinson disease and/or an earlier or later age of developing Parkinson disease can be done according to the protocols set forth in the EXAMPLES section herein and according to well known art methods.

A subject identified as having an increased risk of developing Parkinson disease is a subject whose level of risk of developing Parkinson disease is greater than the level of risk of developing Parkinson disease is for a person lacking the genetic marker of this invention. A subject identified as having a decreased risk of developing Parkinson disease is a subject whose level of risk of developing Parkinson disease is less than the level of risk of developing Parkinson disease is for a person lacking the genetic marker of this invention.

A subject identified as having an earlier age of developing Parkinson disease is a subject who has developed or is likely to develop Parkinson disease at an age that is earlier than the age of a person who lacks the AAO associated genetic marker. In some embodiments, an earlier age of developing PD is before the age of 40. In other embodiments, an earlier age of developing PD is about eight years earlier than the age at which a person (e.g., a family member) has or is likely to develop PD. A subject identified as having a later age of developing Parkinson disease is a subject who has developed or is likely to develop Parkinson disease at an age that is later than the age of onset of PD of a subject who lacks the AAO associated genetic marker. In some embodiments, a later age of developing Parkinson disease is about eight years later than the age at which a person (e.g., a family member) has or is likely to develop PD. In some embodiments, a later age of developing PD can be after the age of 50 or after the age of 55 or after the age of 60.

Furthermore, the present invention provides embodiments that include a method of identifying a subject as having Parkinson disease and/or having an increased risk of developing Parkinson disease and/or having an earlier or later age of developing Parkinson disease, comprising detecting in the subject the presence of a single nucleotide polymorphism in the ubiquitin-specific protease 24 (USP24) gene, wherein the single nucleotide polymorphism is correlated with Parkinson disease and/or an increased risk of developing Parkinson disease and/or an earlier or later age of developing Parkinson disease, thereby identifying the subject as having Parkinson disease and/or having an increased risk of developing Parkinson disease and/or having an earlier or later age of developing Parkinson disease. In this embodiment, the single nucleotide polymorphism in the USP24 gene can be rs487230 (SNP 220), rs683880 (SNP 221), rs667353 (SNP 222), rs594226 (SNP 224), rs1165226 (SNP 227), rs287235 (SNP 230), rs2047422 (SNP 231) and/or any combination of rs487230 (SNP 220), rs683880 (SNP 221), rs667353 (SNP 222), rs594226 (SNP 224), rs1165226 (SNP 227), rs287235 (SNP 230) and rs2047422 (SNP 231).

Also provided herein is a method of identifying a subject as having Parkinson disease and/or having an increased risk of developing Parkinson disease and/or having an earlier or later age of developing Parkinson disease, comprising detecting in the subject the presence of a haplotype in the USP24 gene of the subject comprising the following single nucleotide polymorphisms: rs13312_C (SNP 218_C), rs1043671_T (SNP 219_T), and rs1165226_T (SNP 227_T) or detecting in the subject the presence of a haplotype in the USP24 gene of the subject comprising the following single nucleotide polymorphisms: rs13312_C (SNP 218_C), rs1043671_T (SNP 219_T), and rs1165226_C (SNP 227_C).

Identifying single nucleotide polymorphisms in the USP24 gene and correlating them with Parkinson disease and/or an increase risk of developing Parkinson disease and/or an earlier or later age of developing Parkinson disease can be done according to the protocols set forth in the EXAMPLES section herein and according to well known art methods.

The present invention further provides a method of identifying a subject as having Parkinson disease and/or having an increased risk of developing Parkinson disease and/or having an earlier or later age of developing Parkinson disease, comprising detecting in the subject the presence of a genetic marker of this invention in the leucine rich region kinase (LRRK) gene, wherein the genetic marker is correlated with Parkinson disease and/or an increased risk of developing Parkinson disease and/or an earlier or later age of developing Parkinson disease, thereby identifying the subject as having Parkinson disease and/or having an increased risk of developing Parkinson disease and/or having an earlier or later age of developing Parkinson disease. The LRRK2 gene is linked to an autosomal dominant late-onset form of the disease (Zimprich et al., Neuron 18:601-607, 2004).

Further provided is a method of identifying a subject as having Parkinson disease and/or having an increased risk of developing Parkinson disease and/or having an earlier or later age of developing Parkinson disease, comprising detecting in the subject the presence of a genetic marker of this invention in the TESK2 gene, wherein the genetic marker is correlated with Parkinson disease and/or an increased risk of developing Parkinson disease and/or an earlier or later age of developing Parkinson disease, thereby identifying the subject as having Parkinson disease and/or having an increased risk of developing Parkinson disease and/or having an earlier or later age of developing Parkinson disease.

Additionally, the present invention provides a method of identifying a subject as having Parkinson disease and/or having an increased risk of developing Parkinson disease and/or having an earlier or later age of developing Parkinson disease, comprising detecting in the subject the presence of a genetic marker of this invention in the FLJ14442 gene, wherein the genetic marker is correlated with Parkinson disease and/or an increased risk of developing Parkinson disease and/or an earlier or later age of developing Parkinson disease, thereby identifying the subject as having Parkinson disease and/or having an increased risk of developing Parkinson disease and/or having an earlier or later age of developing Parkinson disease.

In further embodiments, the present invention provides a method of identifying a subject as having Parkinson disease and/or having an increased risk of developing Parkinson disease, comprising detecting in the subject the presence of a single nucleotide polymorphism in the fibroblast growth factor 20 (FGF20) gene, wherein the single nucleotide polymorphism is correlated with Parkinson disease and/or an increased risk of developing Parkinson disease, thereby identifying the subject as having Parkinson disease and/or having an increased risk of developing Parkinson disease. In this embodiment, the single nucleotide polymorphism in the FGF20 gene can be rs1989754, rs1721100, ss20399075, rs6985432, rs11203822, rs108881225, rs1227702208, rs172210282 and/or any combination of rs1989754, rs1721100, ss20399075, rs6985432, rs11203822, rs108881225, rs1227702208 and rs172210282.

Additionally provided herein is a method of identifying a subject as having Parkinson disease and/or having an increased risk of developing Parkinson disease, comprising detecting in the subject the presence of a haplotype in the FGF20 gene of the subject comprising the following single nucleotide polymorphisms: 8p0217_A, rs1989756_G, rs1989754_C, rs1721100_C, and 8p0215_T.

Also provided herein is a method of identifying a subject as having a decreased risk of developing Parkinson disease, comprising detecting in the subject the presence of a haplotype in the FGF20 gene of the subject comprising the following single nucleotide polymorphisms: 8p0217_A, rs1989756_G, rs1989754_G, rs1721100_G, and 8p0215_C.

It is also contemplated in the present invention that a subject can be identified as having Parkinson disease and/or as having an increased risk of developing Parkinson disease and/or an earlier or later age of developing Parkinson disease by detecting the presence of two or more of the genetic markers of this invention in the subject. For example a subject can be screened for two, three, four, five, six or more markers of this invention and two, three, four, five, six or more markers can be detected in the subject, thereby identifying the subject as having Parkinson disease and/or having an increased risk of developing Parkinson disease and/or having an earlier or later age of developing Parkinson disease. Thus, in further embodiments, the present invention provides a method of identifying a subject as having Parkinson disease and/or having an increased risk of developing Parkinson disease and/or having an earlier or later age of developing Parkinson disease, comprising detecting in the subject two or more genetic markers selected, for example from the genetic markers as set forth herein: a) a single nucleotide polymorphism in the HIVEP3 gene, including but not limited to, rs648178 (SNP 13), rs661225 (SNP 19) and/or a combination of rs648178 (SNP 13) and rs661225 (SNP 19); b) a single nucleotide polymorphism in the EIF2B3 gene, including but not limited to, rs263977 (SNP 59), rs263978 (SNP 60), rs263965 (SNP 61), rs1022814 (SNP 62), rs12405721 (SNP 63), rs546354 (SNP 64), rs489676 (SNP 67 and/or any combination of rs263977 (SNP 59), rs263978 (SNP 60), rs263965 (SNP 61), rs1022814 (SNP 62), rs12405721 (SNP 63), rs546354 (SNP 64) and rs489676 (SNP 67); c) a single nucleotide polymorphism in the USP24 gene, including but not limited to, rs487230 (SNP 220), rs683880 (SNP 221), rs667353 (SNP 222), rs594226 (SNP 224), rs1165226 (SNP 227), rs287235 (SNP 230), rs2047422 (SNP 231) and/or any combination of rs487230 (SNP 220), rs683880 (SNP 221), rs667353 (SNP 222), rs594226 (SNP 224), rs1165226 (SNP 227), rs287235 (SNP 230) and rs2047422 (SNP 231); d) a single nucleotide polymorphism in the FGF20 gene, including but not limited to, rs1989754, rs1721100, ss20399075, rs6985432, rs11203822, rs108881225, rs1227702208, rs172210282 and/or any combination of rs1989754, rs1721100, ss20399075, rs6985432, rs11203822, rs108881225, rs1227702208 and rs172210282; e) a functional polymorphism in the tau gene, including but not limited to, IVS3+9A→G, c1632A→G, c1716T→C, c1761G→A, IVS11+34G→A and/or any combination of IVS3+9A→G, c1632A→G, c1716T→C, c1761G→A and IVS11+34G→A; f) a deletion within base pairs 438-477 in exon 3 of the Parkin gene; g) a functional polymorphism in a segment of a chromosome selected from the group consisting of:
a3) a segment of chromosome 2 bordered by D2S2982 and D2S1240;
b3) a segment of chromosome 2 bordered by D2S1400 and D2S2291;
c3) a segment of chromosome 2 bordered by D2S2161 and D2S1334;
d3) a segment of chromosome 2 bordered by D2S161 and D2S2297;
e3) a segment of chromosome 3 bordered by D3S1554 and D3S3631;
f3) a segment of chromosome 3 bordered by D2S1251 and D3S3546;
g3) a segment of chromosome 5 bordered by D5S2064 and D5S1968;
h3) a segment of chromosome 5 bordered by D5S2027 and D5S1499;
i3) a segment of chromosome 5 bordered by D5S816 and D5S1960;
j3) a segment of chromosome 6 bordered by D6S1703 and D6S1027;
k3) a segment of chromosome 6 bordered by D6S1581 and D6S2522;
l3) a segment of chromosome 8 bordered by D8S504 and D8S258;
m3) a segment of chromosome 9 bordered by D9S259 and D9S776;
n3) a segment of chromosome 9 bordered by D9S1811 and D9S2168;
o3) a segment of chromosome 10 bordered by D10S1122 and D10S1755;
p3) a segment of chromosome 11 bordered by D11S4132 and D11S4112;
q3) a segment of chromosome 12 bordered by D12S1042 and D12S64;
r3) a segment of chromosome 14 bordered by D14S291 and D14S544;
s3) a segment of chromosome 17 bordered by D17S1854 and D17S1293;
t3) a segment of chromosome 17 bordered by D17S921 and D17S669;
u3) a segment of chromosome 21 bordered by D21S1911 and D21S1895;
v3) a segment of chromosome 22 bordered by D22S425 and D22S928;
w3) a segment of chromosome X bordered by DXS6797 and DXS1205; and

1x3) a segment of chromosome X bordered by DXS9908 and X telomere; and

any combination of (a3)-(x3), wherein the functional polymorphism is correlated with Parkinson disease or an increased risk of developing Parkinson disease; and h) a functional polymorphism in the LRRK gene, wherein the functional polymorphism is correlated with Parkinson disease or an increased risk of developing Parkinson disease and/or an earlier or later age of developing Parkinson disease; j) a functional polymorphism in the TESK2 gene, wherein the functional polymorphism is correlated with Parkinson disease or an increased risk of developing Parkinson disease and/or an earlier or later age of developing Parkinson disease; k) a functional polymorphism in the FLJ14442 gene, wherein the functional polymorphism is correlated with Parkinson disease or an increased risk of developing Parkinson disease and/or an earlier or later age of developing Parkinson disease; any combination of (a)-(k) above, thereby identifying the subject as having Parkinson disease and/or as having an increased risk of developing Parkinson disease and/or as having an earlier or later age of developing Parkinson disease.

It is also intended that the embodiments of this invention include the detection of a haplotype of this invention, in any combination with the other genetic markers listed herein to identify a subject as having Parkinson disease and/or as having an increased risk of developing Parkinson disease and/or as having an earlier or later age of developing Parkinson disease.

In further embodiments of this invention, the methods can include screening a subject for the presence of a mitochondrial haplogroup associated with a reduced risk of developing Parkinson disease (e.g., haplogroups J and K as described herein in Example 5) and/or for the presence of the SNP 10398G (associated with a reduced risk of developing Parkinson disease), and/or for the presence of SNP 9055A in ATP6 (reduced risk of developing PD in females) and/or for the presence of SNP 13708A in ND5 (reduced risk≧70 group) in addition to screening for other genetic markers of this invention. Also provided is a method of screening a subject for the presence of a mitochondrial haplogroup associated with increased risk of developing Parkinson disease (e.g., haplogroup U in Example 5) in addition to screening for other genetic markers of this invention. These markers can be screened for and/or identified in any combination of genetic markers of this invention.

For example, a subject of this invention can be screened for one or more genetic markers of this invention in the HIVEP3 gene, and/or one or more genetic markers of this invention in the EIF2B3 gene, and/or one or more genetic markers of this invention in the USP24 gene, and/or one more genetic markers of this invention in the FGF20 gene, and/or one or more genetic markers of this invention in the tau gene, and/or one or more genetic markers of this invention in the Parkin gene, and/or one or more genetic markers of this invention in a segment of chromosome described herein in the list designated a3 through x3, as well as any subcombination of genetic markers. A genetic marker of this invention includes a single nucleotide polymorphism, haplotype, deletion, functional polymorphism or other mutation as described herein as associated with Parkinson disease, an increased risk of developing Parkinson disease and/or an earlier or later age of developing Parkinson disease.

A subject of this invention can be identified as having Parkinson disease and/or having an increased risk of developing Parkinson disease and/or having an earlier or later age of developing Parkinson disease by detecting in the subject one or more of the genetic markers of this invention in any combination. For example, the subject can have a genetic marker of this invention in the HIVEP3 gene and a genetic marker of this invention in the tau gene. In other examples, the subject can have a genetic marker of this invention in the EIF2B3 gene, a genetic marker of this invention in the USP24 gene and a genetic marker of this invention in the segment of chromosome described herein in the list designated a3 through x3. In further examples, the subject can have two genetic markers of this invention in the FGF20 gene. In yet other examples, a subject can have one or more genetic markers of this invention in mitochondrial DNA (e.g., haplogroup J or K) that imparts a protective effect and one or more genetic markers of this invention in other genes of this invention that indicate increased risk and/or earlier or later age of developing PD. Thus, it is intended that a subject of this invention can be screened for any combination and any multiplicity of genetic markers of this invention and any combination and any multiplicity of genetic markers of this invention can be detected in a subject

The detection of two or more genetic markers of this invention in a subject can identify the subject as having the same level of increased risk of developing Parkinson disease as the level of increased risk associated with any of the genetic markers of this invention alone and/or the detection of two or more markers of this invention a subject can identify the subject as having a level of increased risk of developing Parkinson disease that is greater than the level of increased risk associated with any of the genetic markers of this invention alone.

In additional embodiments of this invention, methods are provided of identifying a subject with Parkinson disease as having a poor prognosis, comprising detecting in the subject one or more of the genetic markers of this invention. A poor prognosis for Parkinson disease would be identified by one of ordinary skill in the art. A genetic marker of this invention can be correlated with a subject with Parkinson disease having a poor prognosis according to the methods described herein and as are known in the art, in order to identify other subjects with Parkinson disease who are likely to have a poor prognosis.

Additionally, the present invention provides a method of identifying a subject with Parkinson disease as having an increased likelihood of responding effectively to a treatment, comprising: a) correlating the presence of one or more genetic marker of this invention in a test subject effectively responding to the treatment; and b) detecting the genetic marker(s) of step (a) in the subject.

Further provided is a method of identifying a subject with Parkinson disease as having a decreased likelihood of responding effectively to a treatment, comprising: a) correlating the presence of one or more genetic marker of this invention in a test subject who is responding poorly to the treatment; and b) detecting the genetic marker(s) of step (a) in the subject.

A genetic marker of this invention can be correlated with a subject with Parkinson disease having a positive (i.e., effective) response to a particular treatment or a negative response (i.e., ineffective or detrimental) to a particular treatment according to the methods described herein and as are known in the art, in order to identify other subjects with Parkinson disease who are likely to respond effectively to a particular treatment or not likely to respond effectively to a particular treatment. A treatment of this invention is any treatment known in the art or later developed for the treatment of Parkinson disease, for example, including but not limited to chemotherapeutic agents such as levodopa and carbidopa, separately or combined; amantadine hydrochloride, separately or in combination with levodopa and/or carbidopa; anticholinergic agents such as trihexyphenidyl, benzotropine mesylate and procyclidine, separately or in combination with other agents of this invention; selegiline and/or deprenyl separately or in combination with other agents of this invention; dopamine agonists such as bromocriptine, pergolide, pramipexole and andropinirole, separately or in any combination with agents of this invention; catechol-O-methyltransferase (COMT) inhibitors such as tolcapone and entacapone, in combination with levodopa and/or other agents of this invention.

As described herein the present invention includes a method of screening a subject for Parkinson disease and/or increased risk of developing Parkinson disease, comprising detecting the presence or absence of a Parkin gene exon 3 deletion mutation in said subject. The presence of such a deletion mutation indicates that the subject is afflicted with or at risk of developing Parkinson disease. The deletion mutation typically includes a deletion within base pairs 438-477 (e.g., of at least about 10, 20 or 30 or more bases within this region, optionally overlapping with deletions outside of this region). In one embodiment, the deletion mutation is a deletion of base pairs 438 through 477 inclusive. The detection of these markers in combination with other genetic markers of this invention identifies a subject as having Parkinson disease and/or as having an increased risk of developing Parkinson disease.

A further aspect of the present invention is a method of screening for susceptibility to Parkinson Disease in a subject, comprising: determining the presence or absence of an allele of a polymorphic marker in the DNA of the subject, wherein (i) the allele is associated with the phenotype of Parkinson disease, and wherein (ii) the polymorphic marker is within a segment preferably selected from the group consisting of: a segment of chromosome 2 bordered by D2S2982 and D2S1240; a segment of chromosome 2 bordered by D2S1400 and D2S2291; a segment of chromosome 2 bordered by D2S2161 and D2S1334; a segment of chromosome 2 bordered by D2S 161 and D2S2297; a segment of chromosome 3 bordered by D3S1554 and D3S3631; a segment of chromosome 3 bordered by D2S1251 and D3S3546; a segment of chromosome 5 bordered by D5S2064 and D5S1968; a segment of chromosome 5 bordered by D5S2027 and D5S1499; a segment of chromosome 5 bordered by D5S816 and D5S1960; a segment of chromosome 6 bordered by D6S1703 and D6S1027; a segment of chromosome 6 bordered by D6S1581 and D6S2522; a segment of chromosome 8 bordered by D8S504 and D8S258; a segment of chromosome 9 bordered by D9S259 and D9S776; a segment of chromosome 9 bordered by D9S1811 and D9S2168; a segment of chromosome 10 bordered by D10 S1122 and D10S1755; a segment of chromosome 11 bordered by D11S4132 and D11S4112; a segment of chromosome 12 bordered by D12S1042 and D12S64; a segment of chromosome 14 bordered by D14S291 and D14S544; a segment of chromosome 17 bordered by D17S1854 and D17S1293; a segment of chromosome 17 bordered by D17S921 and D17S669; a segment of chromosome 21 bordered by D21 S1911 and D21S1895; a segment of chromosome 22 bordered by D22S425 and D22S928; a segment of chromosome X bordered by DXS6797 and DXS1205; and a segment of chromosome X bordered by DXS9908 and X telomere; the presence of said allele identifying the subject as having an increased risk of developing Parkinson disease. The detection of these markers in combination with other genetic markers of this invention identifies a subject as having Parkinson disease and/or as having an increased risk of developing Parkinson disease.

A still further aspect of the present invention is a method of screening a subject for Parkinson disease, comprising: detecting the presence or absence of a polymorphism or functional polymorphism associated with a gene linked to Parkinson disease; the presence of which identifies the subject as afflicted with or at increased risk of developing Parkinson disease; wherein the gene is the tau gene on chromosome 17. In particular examples, the polymorphism is IVS3+9A>G (an A to G substitution at a location nine base pairs after the end of intron 3); c1632A>G; c1716T>C; c1761G>A; or IVS11+34G>A. The detection of these markers in combination with other genetic markers of this invention identifies a subject as having Parkinson disease and/or as having an increased risk of developing Parkinson disease.

Additionally provided herein is a method of identifying a subject as having Parkinson disease or having an increased risk of developing Parkinson disease and/or having an earlier or later age of developing Parkinson disease, comprising detecting in the subject a functional polymorphism in a gene selected from the group consisting of: a) the synphilin gene and/or the ubiquitin conjugating enzyme (UBE2B) gene on chromosome; b) the NAT1 gene and/or NAT2 gene on chromosome 8; c) the proteasome subunits Z and/or S5 genes and/or the Torsin A and/or Torsin B genes on chromosome 9; and d) the ubiquitin Be gene on chromosome 17, wherein the functional polymorphism is correlated with Parkinson disease or an increased risk of developing Parkinson disease, thereby identifying the subject as having Parkinson disease or having an increased risk of developing Parkinson disease.

As used herein, “a” or “an” or “the” can mean one or more than one. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The term “age at onset” (AAO) or “age of onset” (AOO) refers to the age at which a subject is affected with a particular disease.

The term “Parkinson disease” (PD) as used herein is intended to encompass all types of Parkinson disease. In some embodiments, the term Parkinson disease means idiopathic Parkinson disease, or Parkinson disease of unexplained origin: That is, Parkinson disease that does not arise from acute exposure to toxic agents, traumatic head injury, or other external insult to the brain. In some embodiment, the invention is directed to detecting or screening for late onset Parkinson disease, which refers to Parkinson disease that has a time of onset after the subject reaches about 40 years of age.

“Screening” as used herein refers to methods used to evaluate a subject for PD or an increased risk of developing Parkinson disease and/or of developing PD at an early age (e.g., before the age of 40). It is not required that the screening procedure be free of false positives or false negatives, as long as the screening procedure is useful and beneficial in determining which of those individuals within a group or population of individuals have PD are at increased risk of Parkinson disease, and/or are at increased risk of developing PD at an early age. A screening procedure can be carried out for both prognostic and diagnostic purposes (i.e., prognostic methods and diagnostic methods).

“Prognostic method” refers to methods used to help predict, at least in part, the course of a disease. For example, a screening procedure can be carried out on a subject who has not previously been diagnosed with Parkinson disease, or does not show substantial disease symptoms, when it is desired to obtain an indication of the future likelihood that the subject will be afflicted with Parkinson disease and/or the age at which the subject is likely to develop PD. In addition, a prognostic method can be carried out on a subject previously diagnosed with Parkinson disease or believed or suspected to have PD, when it is desired to gain greater insight into how the disease will progress for that particular subject (e.g., the likelihood that a particular subject will respond favorably to a particular drug or other treatment, and/or when it is desired to classify or separate Parkinson disease patients into distinct and different subpopulations for the purpose of administering a particular type of treatment and/or conducting a clinical trial thereon). A prognostic method can also be used to determine whether and/or how well a subject will respond to a particular drug and/or other treatment.

“Diagnostic method” as used herein refers to methods carried out on a subject to determine if the subject has PD. Such a subject can be someone having no known risk factors, or someone who may be at risk or has previously been determined to be at risk for a particular neurodegenerative disorder due to the presentation of symptoms or the results of a screening test or other type of diagnostic test.

“Functional polymorphism” or “genetic marker” as used herein refers to a change or modification in the nucleotide or base pair sequence of a gene that produces a qualitative or quantitative change in the activity of the gene product (e.g., protein) encoded by that gene (e.g., a change in specificity of activity; a change in level of activity). The presence of a functional polymorphism of this invention can indicate that the subject has PD or is at greater risk of developing PD and/or is at greater risk of developing PD at an early age, as compared to the general population. For example, the patient carrying the functional polymorphism can be particularly susceptible to chronic exposure to environmental toxins that contribute to Parkinson disease. A functional polymorphism of this invention can include but is not limited to mutations, deletions and insertions. In some embodiments, a functional polymorphism of this invention can be a single nucleotide polymorphism.

A “present” functional polymorphism or marker as used herein (e.g., one that is indicative of PD or of a risk factor for Parkinson disease) refers to the nucleic acid sequence corresponding to the functional polymorphism or marker that is found less frequently in the general population relative to Parkinson disease as compared to the alternate nucleic acid sequence or sequences found when such functional polymorphism is said to be “absent.”

“Mutation” as used herein can refer to a functional polymorphism or marker that occurs in less than one percent of the population, and is strongly correlated with the presence of a particular disorder (i.e., the presence of such mutation indicating a high risk of the subject being afflicted with a disease). However, “mutation” as used herein can also refer to a specific site and type of functional polymorphism or marker, without reference to the degree of risk that particular mutation poses to an individual for a particular disease.

“Linked” as used herein refers to a region of a chromosome that is shared more frequently in family members affected by a particular disease than would be expected by chance, thereby indicating that the gene or genes within the linked chromosome region contain or are associated with a marker or functional polymorphism that is correlated to the presence of, or risk of, disease. Once linkage is established association studies (linkage disequilibrium) can be used to narrow the region of interest or to identify the risk-conferring gene associated with Parkinson disease.

“Associated with” when used to refer to a marker or functional polymorphism and a particular gene means that the functional polymorphism or marker is either within the indicated gene, or in a different physically adjacent gene on that chromosome. In general, such a physically adjacent gene is on the same chromosome and within 2, 3, 5, 10 or 15 centimorgans of the named gene (i.e., within about 1 or 2 million base pairs of the named gene). The adjacent gene may span over 5, 10 or even 15 megabases.

A “centimorgan” as used herein refers to a unit of measure of recombination frequency. One centimorgan is equal to a 1% chance that a marker at one genetic locus will be separated from a marker at a second locus due to crossing over in a single generation. In humans, one centimorgan is equivalent, on average, to one million base pairs.

Markers and functional polymorphisms of this invention (e.g., genetic markers such as single nucleotide polymorphisms, restriction fragment length polymorphisms and simple sequence length polymorphisms) can be detected directly or indirectly. A marker can, for example, be detected indirectly by detecting or screening for another marker that is tightly linked (e.g., is located within 2 or 3 centimorgans) of that marker. Additionally, the adjacent gene can be found within an approximately 15 cM linkage region surrounding the chromosome, thus spanning over 5, 10 or even 15 megabases.

The presence of a marker or functional polymorphism associated with a gene linked to Parkinson disease indicates that the subject is afflicted with Parkinson disease or is at risk of developing Parkinson disease and/or is at risk of developing PD at an early age. A subject who is “at increased risk of developing Parkinson disease” is one who is predisposed to the disease, has genetic susceptibility for the disease and/or is more likely to develop the disease than subjects in which the detected functional polymorphism is absent. A subject who is “at increased risk of developing Parkinson disease at an early age” is one who is predisposed to the disease, has genetic susceptibility for the disease and/or is more likely to develop the disease at an age that is earlier than the age of onset in subjects in which the detected functional polymorphism is absent. Thus, the marker or functional polymorphism can also indicate “age of onset” of Parkinson disease, particularly in subjects at risk for Parkinson disease, with the presence of the marker indicating an earlier age of onset for Parkinson disease than in subjects in which the marker is absent. The methods described herein can be employed to screen for any type of idiopathic Parkinson disease, including, for example, late-onset or early-onset Parkinson disease.

Subjects with which the present invention is concerned are primarily human subjects, including male and female subjects of any age or race. Suitable subjects include, but are not limited to, those who have not previously been diagnosed with Parkinson disease, those who have previously been determined to be at risk of developing Parkinson disease and/or at risk of developing PD at an early age, and those who have been initially diagnosed with Parkinson disease or who are suspected of having PD where confirming and/or prognostic information is desired. Thus, it is contemplated that the methods described herein can be used in conjunction with other clinical diagnostic information known or described in the art used in the evaluation of subjects with Parkinson disease or suspected to be at risk for developing such disease.

The present invention discloses methods of screening a subject for Parkinson disease. The method comprises the steps of: detecting the presence or absence of a marker for Parkinson disease, and/or a functional polymorphism associated with a gene linked to Parkinson disease, with the presence of such a marker or functional polymorphism indicating that subject has PD, is at increased risk of developing Parkinson disease and/or is at increased risk of developing PD at an early age.

The detecting step can include determining whether the subject is heterozygous or homozygous for the marker and/or functional polymorphism, with subjects who are at least heterozygous for the functional polymorphism or marker being at increased risk for Parkinson disease and/or of developing PD at an early age. The step of detecting the presence or absence of the marker or functional polymorphism can include the step of detecting the presence or absence of the marker or functional polymorphism in both chromosomes of the subject (i.e., detecting the presence or absence of one or two alleles containing the marker or functional polymorphism). More than one copy of a marker or functional polymorphism (i.e., subjects homozygous for the functional polymorphism) can indicate a greater risk of developing Parkinson disease and/or a greater risk of developing Parkinson disease at an early age, as compared to heterozygous subjects.

The detecting step can be carried out in accordance with known techniques (See, e.g., U.S. Pat. Nos. 6,027,896 and 5,508,167 to Roses et al.), such as by collecting a biological sample containing nucleic acid (e.g., DNA) from the subject, and then determining the presence or absence of nucleic acid encoding or indicative of the functional polymorphism or marker in the biological sample. Any biological sample that contains the nucleic acid of that subject can be employed, including tissue samples and blood samples, with blood cells being a particularly convenient source.

Determining the presence or absence of a particular functional polymorphism or marker can be carried out, for example, with an oligonucleotide probe labeled with a suitable detectable group, and/or by means of an amplification reaction (e.g., with oligonucleotide primers) such as a polymerase chain reaction (PCR) or ligase chain reaction (the product of which amplification reaction can then be detected with a labeled oligonucleotide probe or a number of other techniques). Further, the detecting step can include the step of determining whether the subject is heterozygous or homozygous for the particular functional polymorphism or marker, as described herein. Numerous different oligonucleotide probe assay formats are known which can be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (the entire contents of each of which are incorporated herein by reference). The oligonucleotides can be used to hybridize to the nucleic acids of this invention. In some embodiments, the oligonucleotides can be from 2 to 100 nucleotides and in other embodiments, the oligonucleotides can be 5, 10, 12, 15, 18, 20, 25, 30 35, 40 45 or 50 bases, including any value between 5 and 50 not specifically recited herein (e.g., 16 bases; 34 bases).

Amplification of a selected, or target, nucleic acid sequence can be carried out by any suitable means. See generally, Kwoh et al., Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad Sci. USA 87, 1874-1878 (1990)), the Qβ replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra).

Polymerase chain reaction (PCR) can be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence can be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization (e.g., on a gel). When PCR conditions allow for amplification of all allelic types, the types can be distinguished by hybridization with an allelic specific probe, by restriction endonuclease digestion, by electrophoresis on denaturing gradient gels, or other well known techniques.

Nucleic acid amplification techniques such as the foregoing can involve the use of a probe or primer, a pair of probes or primer, or two pairs of probes or primers that specifically bind to nucleic acid containing the functional polymorphism or marker, but do not bind to nucleic acid that does not contain the functional polymorphism or marker. Alternatively, the probe or primer or pair of probes or primers could bind to nucleic acid that both does and does not contain the functional polymorphism or marker, but produces or amplifies a product (e.g., an elongation product) in which a detectable difference can be ascertained (e.g., a shorter product, where the functional polymorphism is a deletion mutation). Such probes and primers can be generated in accordance with standard techniques from the known sequences of nucleic acid in or associated with a gene linked to Parkinson disease or from sequences that can be generated from such genes in accordance with standard techniques.

It will be appreciated that the detecting steps described herein can be carried out directly or indirectly. Means of indirectly determining allelic type include measuring polymorphic markers that are linked to the particular functional polymorphism, as has been demonstrated for the VNTR (variable number tandem repeats) and the ApoB alleles (Decorter et al., DNA & Cell Biology 9(6):461-69 (1990)), and collecting and determining differences in the protein encoded by a gene containing a functional variant, as described for ApoE4 in U.S. Pat. Nos. 5,508,167 and 6,027,896 to Roses et al.

One form of genetic analysis is centered on elucidation of single nucleotide polymorphisms or “SNPs.” Factors favoring the usage of SNPs as markers of this invention are their high abundance in the human genome (especially compared to short tandem repeats, (STRs)), their frequent location within coding or regulatory regions of genes (which can affect protein structure or expression levels), and their stability when passed from one generation to the next (Landegren et al., Genome Research, 8:769-776 (1998)).

A “SNP” as used herein includes any position in the genome that exists in two variants, with the most common variant occurring less than 99% of the time. In order to use SNPs as widespread genetic markers, it is helpful to be able to genotype them easily, quickly, accurately, and cost-effectively. It is useful to type both large sets of SNPs in order to investigate complex disorders where many loci factor into one disease (Risch and Merikangas, Science 273:1516-1517 (1996)), as well as small subsets of SNPs demonstrated to be associated with known afflictions.

The present invention further provides kits useful for carrying out the methods of the present invention. A kit of this invention will, in general, comprise one or more oligonucleotide probes and/or primers and other reagents for carrying out the methods as described above, such as, e.g., restriction enzymes, optionally packaged with suitable instructions for carrying out the methods. Kits for determining if a subject is or was (in the case of deceased subjects) afflicted with or is or was at increased risk of developing Parkinson disease can include at least one reagent specific for detecting for the presence or absence of at least one functional polymorphism or marker as described herein and instructions for observing that the subject is or was afflicted with or is or was at increased risk of developing Parkinson disease if at least one of the functional polymorphisms is detected. The kit can optionally include one or more nucleic acid probes and/or primers for the amplification and/or detection of the functional polymorphism or marker by any of the techniques described above.

In further embodiments, the present invention provides a method of conducting a clinical trial on a plurality of human subjects or patients. Such methods advantageously permit the refinement of the patient population so that advantages of particular treatment regimens (typically administration of pharmaceutically active organic compound active agents) can be more accurately detected, particularly with respect to particular sub-populations of patients. Thus, the methods described herein are useful for matching particular drug or other treatments to particular patient populations for which the drug or other treatment shows any efficacy or a particular degree of efficacy and to exclude patients for whom a particular drug treatment shows a reduced degree of efficacy, a less than desirable degree of efficacy, or a detrimental effect.

In general, such methods comprise administering a test agent (e.g., active drug or prodrug) or therapy to a plurality of subjects (a control or placebo therapy typically being administered to a separate but similarly characterized plurality of subjects) as a treatment for PD, detecting the presence or absence of at least one mutation or polymorphism or marker of this invention in the plurality of subjects and correlating the presence or absence of the mutation, polymorphism or marker with efficacy or lack of efficacy of the test agent or therapy. The polymorphism or marker or mutation can be detected before, after, or concurrently with the step of administering the test agent or therapy. The correlation of one or more detected polymorphisms or mutations or markers or absent polymorphisms or mutations or markers with the results of the test therapy can then be determined based on any suitable parameter or potential treatment outcome or consequence, including but not limited to: the efficacy of said therapy, lack of side effects of the therapy, etc. The correlation of a particular polymorphism, marker and/or mutation of this invention with any of the tested parameters of the treatment can be determined according to the methods as described herein and as are well known in the art for making such statistical correlations.

The present invention further provides a computer-assisted method of identifying a proposed treatment for Parkinson disease (in a human subject) and identifying patients for whom a particular treatment would be effective, as well as patients for which a particular treatment would not be effective or would be detrimental. The method comprises: (a) storing a database of biological data for a plurality of patients, the biological data that is being stored including for each of said plurality of patients (i) a treatment type, (ii) at least one genetic marker and/or functional polymorphism associated with Parkinson disease, and (iii) at least one disease progression measure for Parkinson disease for which treatment efficacy can be determined; and (b) querying the database to determine the dependence on said genetic marker or functional polymorphism of the effectiveness of a treatment type in treating Parkinson disease, to thereby identify a proposed treatment as an effective treatment for a patient carrying a particular marker for Parkinson disease.

In one embodiment, treatment information for a patient can be entered into the database (through any suitable means such as a window or text interface), genetic marker information for that patient can be entered into the database, and disease progression information can be entered into the database. These steps are then repeated until the desired number of patients has been entered into the database. The database can then be queried to determine whether a particular treatment is effective for patients carrying a particular marker, not effective for patients carrying a particular marker, etc. Such querying can be carried out prospectively or retrospectively on the database by any suitable means, but is generally done by statistical analysis in accordance with known techniques, as described herein and as are well known in the art.

Any suitable disease progression measure can be used, including but not limited to measures of motor function such as tremor measures, rigidity measures, akinesia measures, and dementia measures, as well as combinations thereof. The measures are preferably scored in accordance with standard techniques for entry into the database. Measures are preferably taken at the initiation of the study, and then during the course of the study (that is, treatment of the group of patients with the experimental and control treatments), and the database preferably incorporates a plurality of these measures taken over time so that the presence, absence, or rate of disease progression in particular individuals or groups of individuals may be assessed.

An advantage of the present invention is the relatively large number of genetic markers for Parkinson disease (as set forth herein) that may be utilized in the computer-based method. Thus, for example, instead of entering a single marker into the database for each patient, two, three, five, seven or even ten or more markers may be entered for each particular patient. Note that, for these purposes, entry of a marker includes entry of the absence of a particular marker for a particular patient. Thus the database can be queried for the effectiveness of a particular treatment in patients carrying any of a variety of markers, or combinations of markers, or who lack particular markers.

In general, the treatment type may be a control treatment or an experimental treatment, and the database preferably includes a plurality of patients having control treatments and a plurality of patients having experimental treatments. With respect to control treatments, the control treatment may be a placebo treatment or treatment with a known treatment for Parkinson disease, and preferably the database includes both a plurality of patients having control treatment with a placebo and a plurality of patients having control treatments with a known treatment for Parkinson disease

Experimental treatments are typically drug treatments, which are compounds or active agents that are parenterally administered to the patient (i.e., orally or by injection) in a suitable pharmaceutically acceptable carrier.

Control treatments include placebo treatments (for example, injection with physiological saline solution or administration of whatever carrier vehicle is used to administer the experimental treatment, but without the active agent), as well as treatments with known agents for the treatment of Parkinson disease, such as administration of Levodopa, amantadine, anticholinergic agents, antihistamines, phenothiazines, centrally acting muscle relaxants, etc. See, e.g., L. Goodman and A. Gilman, The Pharmacological Basis of Therapeutics, 227-244 (5^{th} Ed. 1975), the entire contents of which is incorporated herein in its entirety for its teachings of treatment of Parkinson disease.

Administration of the treatments is preferably carried out in a manner so that the subject does not know whether that subject is receiving an experimental or control treatment. In addition, administration is preferably carried out in a manner so that the individual or people administering the treatment to the subject do not know whether that subject is receiving an experimental or control treatment.

Computer systems used to carry out the present invention may be implemented as hardware, software, or both hardware and software. Computer and hardware and software systems that may be used to implement the methods described herein are known and available to those skilled in the art. See, e.g., U.S. Pat. No. 6,108,635 to Herren et al. and the following references cited therein: Eas, M.A.: A program for the meta-analysis of clinical trials, Computer Methods and Programs in Biomedicine, Vol. 53, no. 3 (July 1997); D. Klinger and M. Jaffe, An Information Technology Architecture for Pharmaceutical Research and Development, 14^{th} Annual Symposium on Computer Applications in Medical Care, November 4-7, pp. 256-260 (Washington, D.C. 1990); M. Rosenberg, “ClinAccess: An integrated client/server approach to clinical data management and regulatory approval”, Proceedings of the 21^{st} annual SAS Users Group International Conference (Cary, N.C., Mar. 10-13, 1996). Querying of the database may be carried out in accordance with known techniques such as regression analysis or other types of comparisons such as with simple normal or t-tests, or with non-parametric techniques.

The present invention accordingly provides for a method of treating a subject for Parkinson disease, particularly late-onset Parkinson disease, which method comprises the steps of: determining the presence of a genetic marker for Parkinson disease in said subject; and then administering to said subject a treatment effective for treating Parkinson disease in a subject that carries said marker. The genetic marker is a marker such as described above, but to which a particular treatment has been matched. A treatment is preferably identified for that marker by the computer-assisted method described above. In one a particularly preferred embodiment, the method is utilized to identify patient populations, as delineated by preselected ones of markers such as described herein, for which a treatment is effective, but where that treatment is not effective or is less effective in the general population of Parkinson disease patient (that is, patients carrying other markers, but not the preselected marker for which the particular treatment has been identified as effective).

In further embodiments, the present invention provides a method of identifying a human subject as having Parkinson disease or having an increased risk of developing Parkinson disease and/or having an earlier or later age of developing Parkinson disease, comprising: a) correlating the presence of a single nucleotide polymorphism in the HIVEP3 gene, EIF2B3 gene, the USP24 gene and/or the FGF20 gene with Parkinson disease and/or an earlier or later age of onset of PD; and b) detecting the single nucleotide polymorphism of step (a) in the subject, thereby identifying a subject having Parkinson disease or having an increased risk of developing Parkinson disease and/or having an earlier or later age of developing Parkinson disease.

Also provided herein is a method of identifying a single nucleotide polymorphism in the HIVEP3 gene, the EIF2B3 gene, the USP24 gene and/or the FGF20 gene correlated with Parkinson disease or an increased risk of developing Parkinson disease and/or an earlier or later age of developing Parkinson disease, comprising: a) detecting in a subject with Parkinson disease the presence of a single nucleotide polymorphism in the HIVEP3 gene, the EIF2B3 gene, the USP24 gene and/or the FGF20 gene; and b) correlating the presence of the single nucleotide polymorphism of step (a) with the Parkinson disease in the subject and/or the age of onset of PD in the subject, thereby identifying a single nucleotide polymorphism in the HIVEP3 gene, the EIF2B3 gene, the USP24 gene and/or the FGF20 gene correlated with Parkinson disease or an increased risk of developing Parkinson disease and/or an earlier or later age of developing Parkinson disease.

In addition, the present invention provides a method of correlating a single nucleotide polymorphism in the HIVEP3 gene, the EIF2B3 gene, the USP24 gene and/or the FGF20 gene with Parkinson disease or an increased risk of developing Parkinson disease and/or an earlier or later age of developing Parkinson disease, comprising: a) determining the nucleotide sequence of the HIVEP3 gene, the EIF2B3 gene, the USP24 gene and/or the FGF20 gene of a subject with Parkinson disease; b) comparing the nucleotide sequence of step (a) with the nucleotide sequence of an HIVEP3 gene, the EIF2B3 gene, the USP24 gene and/or the FGF20 gene of a subject without Parkinson disease; c) detecting a single nucleotide polymorphism in the nucleotide sequence of (a); and d) correlating the single nucleotide polymorphism of (c) with Parkinson disease and the age of onset of Parkinson disease.

The present invention is explained in greater detail in the examples that follow. These examples are intended as illustrative of the invention and are not to be taken as limiting thereof.

### EXAMPLES

### Example 1

### Genetic Markers for PD in the FGF20 Gene

The pathogenic process responsible for the loss of dopaminergic neurons within the substantia nigra of Parkinson disease patients is not well understood. However, there is strong evidence to support the involvement of fibroblast growth factor 20 (FGF20) in the survival of dopaminergic neurons. FGF20 belongs to a highly conserved family of growth factor polypeptides that regulate CNS development and function. Additionally, FGF20 is involved in differentiation of rat stem cells into dopaminergic cells. FGF20 is preferentially expressed in rat substantia nigra tissue. The human homologue has been mapped to 8p21.3 to 8p22.

Single nucleotide polymorphisms found in the public record (rs 1989754, rs1989756, and rs1721100) were tested. It was found that the SNP rs1989754 was significantly associated with an increased risk of developing Parkinson disease (Table 1).

Additionally, using DNA sequencing analysis of control DNA, a new polymorphism was discovered, called 8p0215. Association testing demonstrated that this SNP is also highly associated with an increased risk with getting Parkinson disease (Table 1). The “2” allele, which corresponds to the T allele, is the allele associated with increased risk for Parkinson disease. Another SNP, 8p0217, was discovered using the same technique.

Haplotype analysis demonstrated that the h4 haplotype (Table 2) was positively associated with risk for PD, and the h1 haplotype is negatively associated with risk.

The location for 8p215 in the FGF20 cDNA sequence (SEQ ID NO: 1) lies at position 817C>T in the cDNA. The location is shown below. The first base, which is the MET codon, is numbered 1+. The translation and peptide sequence for FGF20 (SEQ ID NO:2) is shown below the coding region.

[image]

[image]

It was determined that SNP rs1989754 lies in the first intron, and 8p0215 lies in the 3′ UTR of FGF20. This SNP is in an intronic area, thus it is best noted by the rs designation. The actual sequence number may change with each number thus one skilled in the art will appreciate that the number may change. The sequence shown below is shown flanking the polymorphism as is characterized as dbSNP rs1989754, has the genomic location Chromosome 8:16,938,312, was characterized by the Sanger Center and was submitted on Oct. 13, 2003. The flanking sequence information and observed SNP are as follows:

| (SEQ ID NO:3) | |
|---|---|
| 5′ flank: | tcctttgaca ttgctagcag gttaactaat agaatggaaa |
| | cttcagctat ggggaaagat cctgggatat tagaaccgga |
| | gagcacccca tctttgtaca gaaaactaag cctcagactg |
| | atgaaggcac tttctagtta cacagctagt gaggaagtca |
| | ttaacaggag agaccctccc gatctagtat cttaacagac |
| | actgccttaa caatcattct cttgtttctt ttaacccctt |
| | ctcttcccag gcactgccgg aggtattctg aaacacgtcc |
| | gtctgtgttc ccacccatat cttctttcgc tttcccattt |
| | cctctttcct aaagtcgata ccaagatact tgctttca |
| Observed: S(c/g) | |
| (SEQ ID NO :4) | |
| 3′ flank: | gttgcacaat ttccaaagag gagcttggct gaagaactag |
| | gcatgctcag tagccgggtg gtcttcctcc tcccccaccc |
| | ctccccccct ttccttttct tttctcaccc acatagaact |
| | taggagctga gggaacctca gacaggtgag ccctacaggt |
| | agcgaatgtg cccacggaaa gttaatctgc tacctcttca |
| | ggtgaacatt tgcaagtctc taggtagaca cgtaaat |

The rs1989754 SNP is located in a HIF1 alpha binding site, which is a known inducer for expression during hypoxia, is shown below (SEQ ID NO:5). The letters in bold (CGTG) are the consensus binding site for HIF1alpha binding. Variation introduced by the rs1989754 SNP disrupts the binding site, with the allele causing an increase in risk with PD disrupting the site, and the allele associated with decreased risk, keeping the site as the consensus sequence.

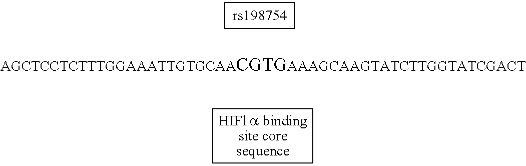

This implies that FGF20 could be induced to express during hypoxia. Using PC12 cells and hypoxic conditions, we demonstrated for the first time that FGF20 is indeed induced by hypoxia.

A Multi-locus genotype PDTsum demonstrates the genotype 22—1,2 is the genotype giving the most significant allele association. (Table 3).

Linkage disequilibrium (LD) analysis demonstrated that the two associated SNPs are in LD with each other (Table 4).

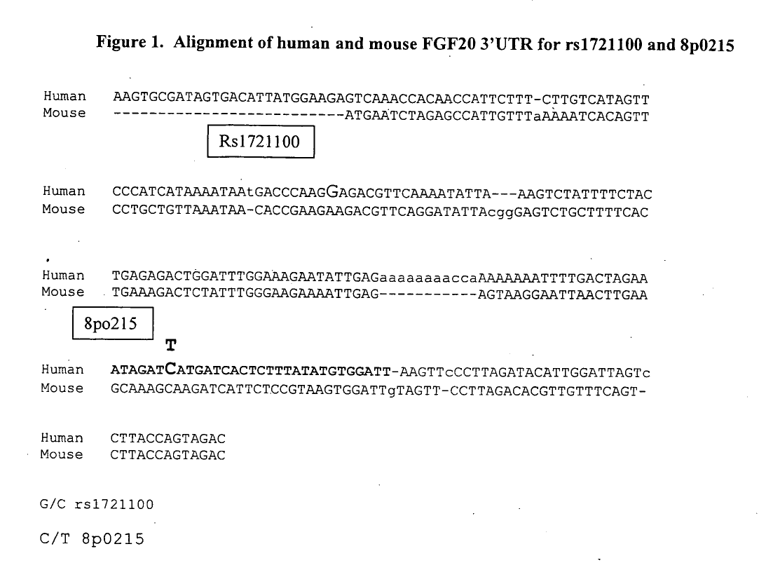

Thus, either or both could illustrate increasing risk for Parkinson disease, either independently or through interaction between them. The SNP 8p0215 we found lies in a highly conserved region of the FGF20 gene, and lies within a PUF binding site, the SNP highlighted in FIG. 1. PUF are proteins that are involved in mRNA stabilization.

In describing the mutations disclosed herein in the novel nucleic acids described herein, and the nucleotides encoding the same, the naming method is as follows: [nucleic acid replaced] [nucleic acid number in sequence of known sequence][alternate nucleic acid]. For example, for the 817^{th} position is cytosine and is replaced with a thymine.

A total of 644 families were genotyped. Of these families, 289 were multiplex families (2 or more affected individuals within a family), and 355 were singleton families (1 affected individual within a family). Exonic, intronic and untranslated regions (UTR) were screened for SNPs by sequencing pools of individuals.

Microarray Gene Expression Study: Total RNA was extracted using TRIzol reagent (Invitrogen, Carlsbad, Calif.) according to the manufacturer's instructions. To label the RNA for hybridization to the microarray chip, 7 μg of total RNA were used for double-stranded cDNA synthesis using the SuperScript Choice System (Gibco BRL Life Technologies, Rockville, Md.) in conjunction with a T7-(dT)-24 primer (Geneset Oligos, La Jolla, Calif.). The cDNA was purified using Phase Lock Gel (3 Prime, Inc., Boulder, Colo.). In vitro transcription was performed to produce biotin-labeled cRNA using a BioArray HighYield RNA Transcript Labeling Kit (Affymetrix, Santa Clara, Calif.) according to the manufacturer's instructions. The biotinylated RNA was cleaned using the RNeasy Mini kit (Qiagen, Valencia, Calif.). See, Lockhart et al., Nat. Biotechnol. 14, 1675 (1996); and Warrington et al., Physiol Genomics 2, 143 (2000).

To probe the microarray, 20 μg of biotinylated cRNA was fragmented and hybridized to microarrays (GeneChip Human Genome U133A array, Affymetrix) using previously described protocols. See, Lockhart et al. The intensity of all features of microarrays was recorded and examined for artifacts (Affymetrix GeneChip® Software v 4.0). O'Dell et al., Eur. J. Hum. Genet 7, 821 (1999). Quantitative gene expression values measured by the average difference between the hybridization intensity with the perfect match probe sets and the mismatch probe sets were then multiplied by a scaling factor to make the mean expression level on the microarray equal to a target intensity of 100. The Affymetrix software to normalize the gene expression levels automatically performs this scaling.

For quality control, all arrays were visually inspected to exclude hybridization artifacts. To control for partial RNA degradation, 3′/5′ end ratios for the housekeeping genes actin and GAPDH were examined. Arrays with high 3′/5′ end ratios suggestive of partial RNA degradation were excluded from further analysis.

Microarray Data Analysis: Since genes with low signal intensity often cause high variability between arrays and Northern blots usually do not confirm positive results for genes with signal intensity less than 500, only genes with average expression intensities of=500 were considered for further analysis. A log₂ (logarithm base 2) was used for data normalization, so data within each chip are in agreement with normal distribution. A two-sample t-test was used to examine whether the gene expression between case and control groups is significantly different. Disease status was randomly assigned to each sample for 1000 times to estimate an empirical p-value for each gene. A nominal significance level of 0.05 was compared with the empirical p-values to declare a result significant.

SNP detection and genotyping: Public domain databases (Japanese JSNP, NCBI dbSNP, and Applied Biosystems) were utilized to identify SNPs located in or near the candidate genes. All other SNPs were genotyped using the assays-on-demand from Applied Biosystems (ABI, Foster City, Calif.). Genomic DNA was extracted from whole blood using the PureGene system (Gentra Systems, Minneapolis, Minn.) and genotyped using the TaqMan allelic discrimination assay. See, Saunders et al., Neurol. 43:1467 (1993); and Vance et al., Approaches to Gene Mapping in Complex Human Diseases, (Wiley-Liss, New York, 1998), Chapter 9.

Association Analysis: All SNPs were tested for Hardy-Weinberg equilibrium (HWE) and linkage disequilibrium (LD) in the affected group (one affected from each family) and the unaffected group (one unaffected from each family). An exact test implemented in Genetic Data Analysis (GDA) program was used to test HWE, in which 3,200 replicate samples were simulated for estimating the empirical P value. See, Zaykin et al., Genetica, 96:169 (1995). The GOLD (Graphical Overview of Linkage Disequilibrium) program was used to estimate the Pearson correlation (r²) of alleles for each pair of SNPs as the measurement of LD. See, Abecasis et al. The higher the r² (0<r²<1), the stronger the LD. In general, r²>0.3 is considered to be a minimum useful value for detecting association with an unmeasured variant related to disease risk by genotyping a nearby marker in LD with that variant See, Ardlie et al., Nat. Rev. Genet. 3:299 (2002). Additionally, the Pedigree Disequilibrium Test (PDT) and GenoPDT were utilized as statistical methods.

The orthogonal model takes information from a general pedigree. It can incorporate covariate effects when necessary. The association between the marker and age-at-onset was identified by testing within family effect, which is equivalent to the additive effect of the marker locus. The empirical p-values were computed through 1000 permutations to avoid false-positive results.

### Example 2

### Screening for Markers Linked to Parkinson Disease

As noted above, the present invention provides a method of screening (e.g., diagnosing or prognosing) for Parkinson disease in a subject. In some embodiments, the method of this invention comprises detecting the presence or absence of a functional polymorphism associated with a gene linked to Parkinson disease as set forth in Table 5.

The present invention can be carried out by screening for markers within particular segments of DNA as described in, for example, U.S. Pat. No. 5,879,884 to Peroutka (the disclosure of which is incorporated by reference herein in its entirety). Examples of suitable segments are provided herein in Table 6.

In general, a method of screening for susceptibility to Parkinson Disease in a subject comprises determining the presence or absence of an allele of a polymorphic marker in the DNA of the patient, wherein (i) the allele is associated with the phenotype of Parkinson disease, and wherein (ii) the polymorphic marker is within a segment set forth in column 3 of Table 6, or within 5, 10, or 15 centiMorgans (cM) of the markers set forth in column 1 of Table 6. The presence of the allele indicates the subject had Parkinson disease or is at increased risk of developing Parkinson disease.

To carry out the methods of this invention, nucleic acid samples can be collected from individuals of a family having multiple individuals afflicted with Parkinson disease. Linkage within that family is then assessed within the regions set forth above in accordance with known techniques, such as have been employed previously, for example, in the diagnosis of disorders such as Huntington's disease, and as described in U.S. Pat. No. 5,879,884 to Peroutka.

Another way to carry out the foregoing methods is to statistically associate alleles at a marker within the segments described herein with Parkinson disease, and use such alleles in genetic testing in accordance with known procedures, such as described for the polymorphism described herein in connection with the tau gene.

Identification of a Parkin Gene Exon 3 Deletion Mutation in Parkinson Disease Families

Multiplex sibship families were collected and a complete genomic screen (N=325 markers; 10 cM grid) was conducted to identify susceptibility genes for familial Parkinson disease (PD).

Individuals with PD (N=379; mean age of onset (AOO)=60.1±12.7 years) and their families (N=175 families with ≧2 members with PD) were collected from 13 sites using strict consensus clinical criteria. This PD dataset is clinically similar to other clinic based populations of Parkinson disease (Hubble et al., Neurology 52:A13 (1999)). Several areas of interest were found including the region containing the Parkin gene. Areas of greatest interest are set forth in Table 5.

Subsequent genetic analysis of these data demonstrated a significant genetic effect in individuals with PD in the chromosome 6 region around the Parkin gene. This effect was strongest in families with at least one member with Parkinson disease onset prior to age 40. Age of onset in this subset (N=89) ranged from 12 to 80 years. This subset was then prioritized for screening of the Parkin gene using denaturing high pressure liquid chromatography (dHPLC). Unique changes in 46 of the 88 individuals screened were identified. Analysis of PCR products of exon 3 of one of the changes revealed a small deletion of bases 438 to 477, present in a homozygous and heterozygous state in at least five different families (range of AOO: 19-53). Examination of these families shows that they have the same 40 bp deletion for exon 3. They were collected from all over the United States of America. Thus this deletion is a relatively common allele in the population, and clearly contributes to PD in the USA, in families not known to have an autosomal recessive inheritance pattern. In fact, the heterozygotes are compound heterozygotes, with a mutation in the other allele in another exon.

Deletions in both copies of the Parkin gene (homozygous deletions) result in a single band that travels farther in on a 2% metaphor gel due to its smaller size. Deletion in only one of the copies (heterozygous deletion) results in two bands. The band that travels farther is the deletion and the other band is the copy of the gene without the deletion (see U.S. Patent Publication No. US-2004-0248092, the entire contents of which are incorporated by reference herein).

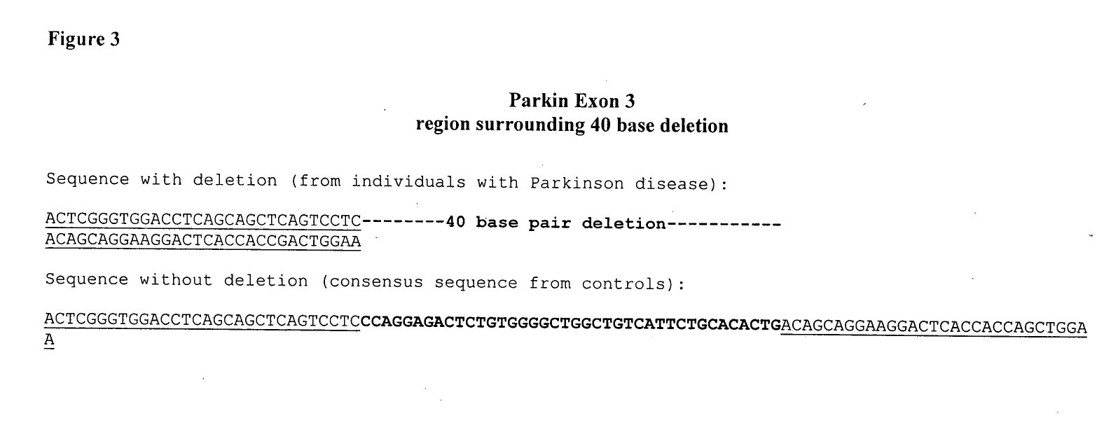

FIG. 3 shows the Parkin gene exon 3 deletion mutation. The upper strand shows exon 3 with the deletion present (SEQ ID NO: 10), as found in individuals with Parkinson disease; the lower strand shows exon 3 without the deletion (SEQ ID NO: 11, consensus sequence from controls). Information such as set forth in FIG. 3 can be used to develop oligonucleotide probes useful for detecting functional polymorphisms in screening procedures for particular functional polymorphisms, as set forth herein.

PCR Screening Procedures

Blood or other biological samples containing DNA are obtained from a subject. DNA is extracted from these samples using conventional techniques. Polymerase chain reaction is performed on the genomic DNA of the subject using the primers for Parkin Exon 3 described in Kitada et al. (Nature 392:605 (1988); the disclosure of which is incorporated herein by reference in its entirety), as follows:

(SEQ ID NO:12)

| | |
|---|---|
| forward | (5′-3′) ACATGTCACTTTTGCTTCCCT |

(SEQ ID NO:13)

| | |
|---|---|
| reverse | (5′-3′) AGGCCATGCTCCATGCAGACTGC |

The shortened PCR product produced by the 40 base pair exon 3 deletion mutation (bp438-477) (numbering based upon the cDNA of Kitada et al.) can be detected from the amplification products of such primers by a variety of techniques. For example, agarose gel separation of the PCR products in which two bands would be obtained can be used, with the smaller molecular weight band being the one containing the deletion. The size of the deletion can be measured using a molecular weight standard. In the alternative, denaturing high performance liquid chromatography (DHPLC) can be used, in which a distinct peak representing the deletion is detected that comes off the column earlier than control peaks. Identification of this specific deletion would require subsequent sequencing of the PCR product.

Parkin Mutations and Idiopathic Parkinson Disease

The marker D6S03, parkin intron 7, was found in further screening of 174 linked early onset (n=18) and late onset (n=156) Parkinson disease families to be strongly linked to Parkinson disease, with a peak Lod score of 5.0.

Familial and sporadic PD cases were screened for parkin mutations, unselected for age at onset or inheritance pattern. Samples were from 88 affected individuals (mean age of onset: 38.6±14.2; selected from 57 families containing individuals with age of onset less than 40; 83% with a reported family history of PD) as well as pools of affected individuals from 308 families (mean age of onset 54.4±13 years; selected individual with earliest age of onset from each family; pools of 5 samples; 97% with reported family history of PD).

A two stage mutation screening strategy was employed, with exons amplified using PCR primers from Hattori et al. (Ann. Neurol. 44:935-41 (1998)). Products were initially screened using denaturing high-pressure liquid chromatography (DHPLC), and DHPLC abnormalities were studied further by sequencing. Results are summarized in Table 7 (numbering based on the cDNA of Kitada et al.).

Ten distinct mutations were detected, only three of which were previously reported. Two mutations (exon 7, Asp>Asn and exon 3, Ala>Glu) were detected only in late-onset families.

The mutations noted in Table 7 can be used to carry out the methods described herein.

Genomic Screening for Additional Parkinson Disease Markers

To identify additional regions of the genome with genes contributing to idiopathic PD, we performed a complete genomic screen for linkage analysis in 174 PD families containing at least one affected relative pair.

Family Ascertainment. The Duke Center for Human Genetics (DCHG)/GlaxoSmithKline/Deane Laboratory Parkinson Disease Genetics Collaboration is a 13-center effort established to ascertain multiplex (two or more participating individuals diagnosed with PD) families for genetic studies of PD. Family history of PD was documented for each family by conducting a standard interview with the proband or a knowledgeable family informant. The results of this interview were used to generate pedigrees documenting the extent of family history of PD out to three degrees of relationship (1^{st} cousins). Consensus diagnostic and exclusion criteria were developed by all participating clinicians prior to beginning ascertainment of families. All participants are examined prior to enrollment in the study by a board-certified neurologist or a physician assistant trained in neurological disease and supervised by a neurologist. Participants are classified as affected, unclear, or unaffected based on neurological exam and clinical history. Affected individuals possess at least two cardinal signs of PD (rest tremor, bradykinesia, and rigidity) and have no atypical clinical features or other causes of parkinsonism. Unclear individuals possess only one sign and/or have a history of atypical clinical features, and unaffected individuals have no signs of PD. Excluded from participation are individuals with a history of encephalitis, neuroleptic therapy within the year prior to diagnosis, evidence of normal pressure hydrocephalus, or a clinical course with unusual features, suggestive of atypical or secondary parkinsonism. Age at onset was self-reported, defined as the age at which the affected individual could first recall noticing one of the primary signs of PD. Physician and patient observations of response to levodopa therapy were used to classify individuals as responsive or non-responsive to levodopa. Individuals for whom levodopa was of uncertain benefit or who never received levodopa therapy were classified as having unknown levodopa response. To ensure diagnostic consistency across sites, clinical data for all participants was reviewed by a clinical adjudication board, consisting of a board certified neurologist with fellowship training in movement disorders, a dually board-certified neurologist and Ph.D. medical geneticist, and a certified physician assistant. All participants gave informed consent prior to venipuncture and data collection according to protocols approved by each center's institutional review board.

The first 174 families with sampled affected relative pairs were included in this initial genomic screen. The number of sampled affected family members and affected relative pairs is presented in Table 8. The families contained an average of 2.3 affected individuals and an average of 1.5 affected relative pairs per family. While the majority of the affected relative pairs were affected sibpairs (185/260), there were 75 other affected relative pairs (avuncular, cousin, and parent-child pairs) in the data set. These data illustrate that, while smaller family aggregates without a recognizable mode of inheritance were studied, families were often multigenerational in structure and that the study was not limited to affected sibpairs.

All families studied were Caucasian. Overall, 870 individuals (an average of 5 per family) from these families were studied: 378 affected with PD (43%), 379 unaffected (44%), and 113 with unclear affection status (13%). In affected individuals, the mean age at onset of PD was 59.9±12.6 years (range: 12-90), and the mean age at examination was 69.9±10.2 years (range: 33-90). Mean age of examination in unaffected individuals was 67.1±12.9 years (range 31-96), and mean age of examination in those with unclear affection status was 72.1±11.6 years (range 49-90).

Molecular Analysis. Genomic DNA was extracted from whole blood using Puregene© in methods previously described (Vance, in Approaches to Gene Mapping in Complex Human Diseases, Haines and Pericak-Vance, Eds., Wiley-Liss, New York, 1998, Chap. 8). Analysis was performed on 344 microsatellite markers with an average spacing of 10 cM. Genotyping was performed by the FAAST method previously described (Vance & Ben Othmane, in Approaches to Gene Mapping in Complex Human Diseases, Haines and Pericak-Vance, Eds., Wiley-Liss, New York, 1998; Chap. 9). Systematic genotyping errors were minimized using a system of quality control checks with duplicated samples (Rimmler et al., Am. J. Hum. Genet. 65:A442 (1999)). On each 96-well PCR plate, two standard samples from CEPH families are included and 6 additional samples are duplicates of samples either on that plate or another plate in the screen. Laboratory technicians are blinded to the location of these QC samples to avoid bias in interpretation of results. Automated computer scripts check each set of genotypes submitted by the technician for mismatches between the duplicated samples; mismatches are indicative of potential genotype reading errors, mis-loading of samples, and sample mix-ups.

As an additional quality control measure, potential pedigree errors were checked using the program RELPAIR (Boehnke & Cox, Am. J. Hum. Genet. 61:423 (1997)), which infers likely relationships between pairs of relatives using IBD sharing estimates from a set of microsatellite markers.

Statistical Analysis. Data analysis consisted of a multianalytical approach consisting of both parametric lod score and non-parametric affected relative pair methods. Maximized parametric lod scores (MLOD) for each marker were calculated using the VITESSE and HOMOG program packages (O'Connell & Weeks, Nat. Genet. 11:402 (1995); Ott, Analysis of Human Genetic Linkage. (The Johns Hopkins University Press, Baltimore, Ed. 3, 1999); The MLOD is the lod score maximized over the two genetic models tested, allowing for genetic heterogeneity. Dominant and recessive low-penetrance (affecteds-only) models were considered. Prevalence estimates for PD range from 0.3% in individuals aged 40 and older to 2.5% in individuals aged 70 and older [Tanner & Goldman, Neurol. Clin. 14:317 (1996)]. Based on these prevalence estimates and allowing for age-dependent or incomplete penetrance, disease allele frequencies of 0.001 for the dominant model and 0.20 for the recessive model were used. Marker allele frequencies were generated from over 150 unrelated Caucasian individuals. Multipoint non-parametric lod scores (LOD*) were calculated using GENEHUNTER-PLUS software (Kong & Cox, Am. J. Hum. Genet. 61:1179 (1997)) and sex-averaged intermarker distances from the Marshfield Center for Medical Genetics genetic linkage maps were used in these analyses. In contrast to non-parametric linkage approaches which consider allele sharing in pairs of affected siblings [Risch, Am. J. Hum. Genet. 46:222 (1990)], GENEHUNTER-PLUS considers allele sharing across pairs of affected relatives (or all affected relatives in a family) in moderately sized pedigrees. We selected GENEHUNTER-PLUS to take advantage of the additional power contributed to the sample by the 75 affected relative pairs that would be ignored by an affected sibpair analysis. Due to computational constraints on pedigree size, 27 unaffected individuals from 12 families were omitted from GENEHUNTER-PLUS analysis.

Due to the potential genetic heterogeneity in this sample, a priori we stratified the data set in two ways. The first was to divide the sample by age at onset. Families with at least one member with early-onset (<40 years (Golbe, Neurology 41:168 (1991))) PD (n=18) were considered separately from the rest of the (late-onset) families (n=156). Mean age at onset in the early-onset families was 39.7 years (range: 12-66), while mean age at onset in the late-onset families was 62.7 years (range: 40-90). The two age of onset groups were similar with respect to average family size and structure. Also, nine families (all late-onset) contained at least one affected individual who was determined to be non-responsive to levodopa therapy; these families were considered separately from the rest of the late-onset families (n=147).

The intent of an initial complete genomic screen is to identify regions of the genome likely harboring susceptibility loci for more thorough analysis. Because genetic heterogeneity likely reduces the power to detect statistically significant evidence of linkage using the traditional criterion of a lod score>3, we chose a more liberal criterion of a lod score>1 in the overall sample for consideration of a region as interesting and warranting initial follow-up. Regions were then prioritized into two groups for efficient laboratory analysis: regions generating lod scores>1 on both two-point and multipoint analyses were classified as priority 1, while regions with lod scores>1 on only one test were designated priority 2. While this approach may increase the number of false-positive results that are examined in more detail, it decreases the more serious (in this case) false-negative rate.

Genetic regions generating LOD*>1 are listed in Table 9. Markers on chromosomes 5p, 5q, 8p, 9q, 14q, 17q, and Xq generated interesting two-point lod scores (MLOD>1) in the overall sample of 174 families. Four of these regions also produced multipoint LOD* scores>1 and were classified as priority 1 for follow-up. The strongest evidence for linkage in the overall data set was on chromosome 8p (MLOD=2.01 at D8S520; LOD*=2.22). Other regions with interesting two-point and multipoint results were 5q (MLOD=2.39 at D5S816; LOD*=1.5), 17q (MLOD=1.92 at D17S921; LOD*=2.02), and 9q (MLOD=1.59 at D9S2157; LOD*=1.47). Three regions with two-point lod scores>1 (5p, 14q, Xq) did not have multipoint LOD*>1 and were designated priority 2 for follow-up.

Two-point results obtained from the subset of 156 late-onset families were essentially similar. In addition to the seven interesting regions identified in the overall sample, lod scores were >1 at markers on chromosomes 21p and 22q. The strongest result in this subset was on 17q (MLOD=2.05 at D17S1293; LOD*=2.31), followed by 8p (MLOD=1.96 at D8S520; LOD*=1.92), and 9q (MLOD=1.36; LOD*=1.4). The other six regions with interesting two-point results (5p, 5q, 14q, 21p, 22q, and Xq) generated multipoint LOD*<1.

In the subset of 18 early-onset families, only two regions identified in the overall sample (5q and 17q) generated interesting two-point results. Five additional regions (2q, 6q, 10q, 11q, and 12q) generated lod scores>1 in this subset. A highly significant result was obtained at D6S305 (MLOD=5.07; LOD*=5.47). An additional region with interesting two-point and multipoint results was identified on chromosome 11q (MLOD=1.22 at D11S4131; LOD*=1.53). Multipoint LOD* scores on chromosomes 2q, 5q, 10q, 12q, and 17q were less significant (LOD*<1).

Examination of the nine families containing affected individuals whose PD was not responsive to levodopa therapy produced several novel results. In addition to supporting linkage to regions on chromosomes 5q, 9q, 17q, and 22q indicated by the overall late-onset subset, these nine families also implicated regions on chromosomes 3q, 6q, 20p, and a second region on 9q. The strongest results in this subset were obtained from the multipoint analysis of chromosome 9q (MLOD=0.98 at D9S2157; LOD*=2.59). Analysis of the 147 remaining late-onset families separately did not generate any significantly different two-point results from the analysis of all 156 late-onset families.

In summary, these results provide very strong evidence that several genes influence the development of familial PD and that age at onset and levodopa response pattern influence the evidence for linkage to each gene. In contrast to recent contentions that most late-onset PD is caused by environmental factors (Tanner et al., JAMA 281:341 (1999)), these data suggest that several genes may influence the development of late-onset familial PD.

### Example 3

### Association of tau with Late-Onset Parkinson Disease

To examine the role of the tau gene in PD, five polymorphisms in the tau gene were tested for association with PD in a sample of PD families.

Study Subjects. The sample consists of 1056 individuals in 235 families (N=17). Most families in this study are discordant sibships (at least one affected and one unaffected sibling) without parental samples (N=156). A smaller number are nuclear families with at least one affected individual with both parents (N=40) or only one parent (N=3) sampled. The remaining families are more complex, containing more than a single nuclear family or sibship (N=36). This data set contains many of the families used in the PD genomic screen described herein and some additional families. Only families with at least one affected individual with either both parents sampled or at least one unaffected sibling sampled were included to provide more flexibility in the association analyses. When possible, unaffected siblings who were older at age of exam than the age of onset of their affected siblings were sampled. The mean age of onset in affected individuals in the sample is 57.5 years, and the mean age of unaffected individuals is 66.8 years (Age at onset was self-reported, defined as the age at which the affected individual could first recall noticing one of the cardinal signs of PD).

Excluded from participation are individuals with a history of encephalitis, neuroleptic therapy within the year prior to diagnosis, evidence of normal pressure hydrocephalus, or a clinical course with unusual features, suggestive of atypical or secondary parkinsonism. To exclude PSP, FTDP, and other parkinsonian conditions from the PD affected group, all subjects in the PD affected group had to meet strict clinical criteria. All subjects affected with PD in this study had asymmetric motor symptoms at onset, no postural instability with falls early in the disease course, and no supranuclear down- or lateral-gaze palsy. The presence of any one of these exclusion criteria was sufficient to prevent inclusion in the PD affected group, and excluded subjects with clinical features of PSP and other atypical parkinsonian syndromes. Subjects with FTDP were excluded from the PD affected group by clinical criteria requiring the absence of dementia at onset and the presence of asymmetric onset of motor symptoms. Other parkinsonian syndromes were screened by additional clinical criteria such as absence of severe autonomic neuropathy or signs of significant cerebellar dysfunction (multiple system atrophy, MSA); absence of abrupt symptom onset or of a stepwise course (vascular parkinsonism); and absence of unilateral dystonia with apraxia or cortical sensory loss (cortical-basal ganglionic degeneration, CBGD).

Family history of PD was documented for each family by conducting a standard interview with the proband or a knowledgeable family informant. The results of this interview were used to generate pedigrees documenting the extent of family history of PD out to three degrees of relationship (first cousins).

Molecular Analysis. Five SNPs in tau, previously tested for association with PSP (Baker et al., Hum. Mol. Genet. 8:711 (1999)), were chosen for analysis of association in the PD family sample. Two SNPs are intronic: one in intron 3 (SNP 3) and one in intron 11 (SNP 11). The other three SNPs chosen are all in exon 9 (SNPs 9i, 9ii, 9iii). The dinucleotide repeat polymorphism between exons 9 and 10 was also tested (Conrad et al., Ann. Neurol. 41:277 (1997)).

DNA was extracted from whole blood using Puregene kits (Gentra Systems, Minneapolis, Minn.) by the Center for Human Genetics DNAbanking Core. SNPs were genotyped using a modification of the gel-based Oligonucleotide Ligation Assay (OLA) (Eggerding et al., Hum. Mutat. 5:153 (1995)), which consists of an initial multiplex PCR amplification followed by a subsequent ligation (PCR amplification was performed in 10 μL reactions (30 ng DNA, 1X Gibco PCR buffer, 0.6 mM dNTP, 3.0 mM Mg, 0.5 U Gibco Platinum Taq and 0.04 μg forward and reverse primers) using MJ PTC200 or Primus96Plus (MWG-Biotech, Ebersberg, Germany) thermocyclers for 40 cycles (94° C 4 min.; 5×[94° C./30 sec., 55° C./30 sec, 72° C./30 sec]; 20×[94° C./5 sec., 55° C./30 sec, 72° C./45 sec];15×[94° C./5 sec., 55° C./45 sec, 72° C./80 sec]; 72° C./7 min) followed by a 30 minute incubation at 94° C. to heat kill the enzyme. Two microliters of the PCR reaction mix were transferred and dried prior to being resuspended in 10 μL of Ligation mix [1X Taq DNA ligase buffer, 4 U Taq DNA thermostable ligase] (New England BioLabs, Beverly, Mass.). Allele specific probes were fluorescently labeled using Fam or Cy3 and common probes were phosphorylated on the 5′ end. Ligations were performed in a MJ PTC200 or Primus96Plus thermocycler (40X[94° C., 20 sec; 50° C., 1 min]). Reactions were stopped with the addition of 20 μl of loading/stop dye (98% deionized formamide, 10 mM EDTA, 0.025% xylene cyanol, 0.025% bromophenol blue). Approximately 4 μl of each sample was loaded onto a 6% polyacrylamide gel, run for approximately 40 minutes, and scanned on a Hitachi FMBio II fluorescence static scanner. Images were analyzed using Biolmage software. Genotyping of the microsatellite marker was performed by fluorescence imaging using the FASST method previously described (Vance & Ben Othmane, Methods of Genotyping, Haines and Pericak-Vance, Eds., John Wiley & Sons, Inc., New York, 1998). To ensure correct OLA genotyping, representative OLA genotypes were checked for accuracy using sequencing (CEQ2000XL). Table 10 shows PCR primers and OLA probes for SNPs used in this study.

Quality control was conducted by the Center for Human Genetics Data Coordinating Center (DCC) using a set of internal QC samples to which the technicians were blinded (Rimmler et al., Am. Soc. Hum. Gen. 63:A240 (1998)). As an additional level of QC for our candidate gene analyses, each pair of markers within each gene was tested for recombination using Fastlink (Cottingham et al., Am. J. Hum. Gen. 53:252 (1993); Schaffer et al., Hum. Hered. 44:225 (1994)). All individuals in families showing evidence of recombination between markers were checked for genotype misreads. Because four of these SNPs have been reported elsewhere (Baker et al., Hum. Mol. Genet. 8:711 (1999)) to be in strong linkage disequilibrium, genotypes of individuals showing evidence of haplotypes that were not expected were also checked. In each case, rereads or direct sequencing resolved the recombination or haplotype discrepancy.

Statistical Analysis. Two complementary methods for association analysis that are appropriate for this family data were used: (1) the pedigree disequilibrium test (PDT) (Martin et al., Am. J. Hum. Genet. 67, 146 (2000)), and (2) the likelihood ratio test (LRT) implemented in the program Transmit (Clayton, Am. J. Hum. Gen. 65:1170 (1999)). A version of the PDT based on the PDT-sum statistic was used (Martin et al., Am. J. Hum. Gen. 68:1065-1067 (2001)). The robust variance estimator was used in the LRT of Transmit to assure validity as a test of association in sibships of arbitrary size. The data set used for association analyses consists of few extended pedigrees, thus the Transmit analysis is reported based on all nuclear families. P-values for a global test of significance were computed using the chi-squared distribution with h-1 degrees of freedom, where h is the number of distinct haplotypes observed (h=2 for single-locus tests). SNPs were analyzed individually using both methods. Haplotype analysis was also conducted, testing for association with haplotypes including multiple SNPs, using Transmit (inferred haplotypes with frequencies<0.01 were combined with more frequent haplotypes).

To further refine the analyses, two criteria were considered for stratification. Families were classified as family-history positive if a relative of the proband is reported to be affected with PD, or family-history negative if there was no report of PD in the family other than the proband. Families were classified as early-onset if there was at least one individual with age of onset<40 years and late-onset if all individuals had age of onset≧40 years. Nine of the early-onset families have known mutations in the parkin gene. To improve homogeneity in the sample, the early-onset families excluding those with known parkin mutations were also analyzed. The PDT and Transmit test were conducted using families within each stratum.

A single affected and unaffected individual were selected at random from each family for tests of Hardy-Weinberg disequilibrium (HWD) and linkage disequilibrium between markers. Analysis was conducted in the affected sample and unaffected sample separately. The tests implemented in the Genetic Data Analysis Program (version 1.0 d16b) were used (Lewis & Zaykin, Genetic Data Analysis: Computer program for analysis of allelic data. 1.0(d15) (2000)). P-values were estimated using 3200 permutations.

Table 11 shows p-values for single-locus association analyses using PDT and Transmit. The Transmit test was significant (p<0.05) for three of the markers (SNPs 3, 9i and 11). The PDT shows the same trend as the Transmit tests, giving marginally significant results at the same markers. For each marker, it is the more common allele (allele 2) that is positively associated with PD in our sample. Maximum likelihood estimates for allele frequencies of the positively associated allele, from Transmit, are shown in Table 11. For PDT, the positively associated allele occurs more frequently in affected siblings than in unaffected siblings. For Transmit, the positively associated allele is transmitted from parents to affected individuals more frequently than expected. For each marker, PDT and Transmit both show the same allele to be positively associated. The high frequency of the allele at SNP 9iii (Table 11) offers an explanation for why no association was detected. If the positively-associated allele is at high frequency in the population, it will be difficult to detect the association since there cannot be a large difference between the allele frequency in the population and in the affecteds, even if the allele has a frequency of 1.0 in the affecteds.

As has been reported elsewhere (Baker et al., Hum. Mol. Genet. 8:711 (1999)), there was considerable linkage disequilibrium between the markers. In all individuals, the two haplotypes H1 and H2 observed by Baker et al. were the only haplotypes directly observed for SNPs 3, 9i, 9ii and 11. There was no evidence of the existence of other haplotypes for these four markers in our sample. P-values smaller than 1/3200 were estimated for all combinations of these markers. For SNP 9iii, the rare allele occurs almost exclusively with common haplotype, suggesting other haplotypes are old and this allele at 9iii arose more recently on the common Hi haplotype. Significant linkage disequilibrium was not detected between SNP 9iii and the other four markers in either the affected or the unaffected samples. No evidence for deviation from Hardy-Weinberg equilibrium was found in affecteds or unaffecteds for any of the markers.

Table 12 shows the results of the haplotype association analysis with Transmit for the five-locus haplotypes. Only three common haplotypes were observed for the five loci. Individual p-values for the two most common haplotypes were significant with p<0.01. The haplotype carrying alleles 11121 (at SNPs 3, 9i, 9ii, 9iii and 11, respectively) is significantly under-transmitted to affected individuals, while the haplotype carrying alleles 22222 is significantly over-transmitted to affected individuals. Interestingly, the 22222 haplotype corresponds to the Hi haplotype previously associated with PSP (Baker et al., supra). There is no evidence for association with the H1 sub-haplotype carrying allele 1 at 9iii, suggesting that the putative susceptibility allele may occur with increased frequency on the H1-haplotype carrying allele 2 at 9iii.

Table 13 shows results for stratified analyses using Transmit. The single-locus and haplotype association tests in family-history-positive families are close to the p-values in the overall sample. The tests in family-history-negative families are not significant for any of the comparisons. The level of significance tends to decrease in the early- and late-onset families relative to the whole sample, however the results in the late-onset subset are marginally significant (p<0.1) for three of the SNPs and the five locus haplotype. In general, significance decreased for tests in the early-onset families when families with known parkin mutations were excluded. However, this subset contains only 30 families, thus it would be quite difficult to detect an association, even if the sample is more homogeneous.

A dinucleotide repeat polymorphism, previously associated with PSP (Baker et al., supra), positioned between exons 9 and 10 in the tau gene, was also examined for association with PD. The repeat was typed in a set of 249 multiplex PD families, ascertained for family-based linkage studies as described above, which overlaps with the data set used for SNP analyses. A significant association was found with the LRT of Transmit (global test p=0.0286), with the common allele, a0, being significantly overtransmitted to affected individuals and allele a3 being significantly undertransmitted. These results are consistent with the findings of Baker et al., supra for PSP, though not as significant, and further supports the recent report by Pastor et al. of a difference in a0 allelic frequency between PD patients and controls (Neurol. 47:242 (2000)).

### Example 4

### Identification of Risk and Age-at-Onset Genes on Chromosome 1p in Parkinson Disease

In this study, we present the application of the genomic convergence approach combined with “iterative association mapping” to screen a dense map of SNPs in the 1 LOD score region of the chromosome 1p linkage peak. In this region, there are 199 Ensemb1 genes (NCBI build 35) and 4,924 SNPs with a minor allele frequency (MAF) of >10% in the Caucasian population. Using this approach, we have identified several genes that show association with AAO, and surprisingly, one gene that shows association with risk.

Patients and Families. Affected individuals and family members were collected by the Morris K. Udall Parkinson Disease Research Center of Excellence (PDRCE) located within the Duke Center for Human Genetics (DCHG), and the 13 centers of the Parkinson Disease Genetics Collaboration (PDGC) (Scott et al. 2001). A standard clinical evaluation involves a neurological examination including the Unified Parkinson Disease Rating Scale (UPDRS) (Fahn et al. 1987). A rigorous clinical assessment was performed by all participating clinicians in order to provide a clear diagnosis of PD and to exclude any individuals who displayed atypical features of Parkinsonism (Scott et al. 2001; Hubble et al. 1999). Individuals characterized as “affected” showed at least two of the cardinal signs of PD (resting tremor, bradykinesia, and rigidity). AAO for affected individuals was defined as the age at which an affected individual first noticed one of the cardinal signs of PD. Participants characterized as “unaffected” demonstrated no signs of the disease and participants categorized as “unclear” showed only one cardinal sign and/or atypical features. All participants signed informed consents prior to blood and data collection. Institutional review boards at each participating center approved study protocols and consent forms.

The data set consists of multiplex (N=267) and singleton (N=361) white families. We defined singleton and multiplex families based on the total number of parent-child triads and discordant sibpairs (DSP) in a family that can contribute to the association test. Singleton families have only one group (either triad or DSP) contributing to the association test, that is, only one affected individual, with either the parent (affected or unaffected) or unaffected sibling sampled in addition to the affected individual. Multiplex families have at least two groups (triads or DSPs) contributing to the association test, that is, they have at least two affected siblings sampled in the family. Families with Parkin mutation carriers were excluded from this study. The multiplex data set includes 609 affected individuals (average AAO±SD=61.0±11.6 yrs; range: 14-90 yrs; 58.8% males) and 666 unaffected individuals (42.8% males). The singleton families include 391 affected individuals (average AAO±SD=55.5±13.0 yrs; range: 15-85 yrs; 69% males) and 356 unaffected individuals (42.7% males).

DNA extraction and genotyping. DNA samples were prepared and stored by the DCHG DNA bank core. Genomic DNA was extracted from whole blood using the PureGene system (Gentra Systems Autopure LS). A total of 284 SNPs (17) were genotyped using Applied Biosystems (ABI) Assays-on-demand (AoD) or Assays-by-design (AbD), or with the use of primers and probes designed using the ABI Primer Express 2.0 software. The SNPs were chosen first on the basis of their location (e.g., average 100 kilobases [kb] distance between SNPs), and then on the basis of frequency, in order to capture a wide range of frequencies among all selected SNPs. The TaqMan allelic discrimination assay was used to genotype all SNPs. The PCR amplification was performed in 5 μl reactions (2.6 ng dried DNA, 1X TaqMan® universal PCR master mix from ABI, 1X genotyping mix for AoDs and AbDs or 900 nM of each primer and 200 nM of each probe for self-designed assays). PCR was performed using the GeneAmp PCR system 9700 thermocyclers (ABI) and using a 40-cycle program [95° C./10 min; 40X (95° C./15 s, Tₘ/1 min), where Tₘ is 60° C. for AoDs and AbDs and ranges from 58° C. to 64° C. for self-designed assays]. The fluorescence generated during the PCR amplification was read using the ABI Prism 7900HT sequence detection system and analyzed with SDS software (ABI).

Stringent quality control measures were taken to ensure data consistency. Internal controls consisted of 24 duplicated individuals per 384-well plate. In addition, two samples from the Centre d'Étude du Polymorphisme Humain (CEPH) were plated eight times per plate to assure plate-to-plate consistency. All genotypers were blinded to these internal controls. Quality control samples were compared in the DCHG Data Coordinating Center. Data were stored and managed by the PEDIGENE® system (Haynes et al. 1995). In order to pass quality control, genotyping plates must have retained a 100% match for quality control samples and must have at least 95% overall efficiency.

Candidate genes derived for the genomic convergence approach. Two independent gene expression studies on human midbrain tissues from PD patients and normal controls, by use of microarray and serial analysis of gene expression (SAGE) technologies, were conducted as a part of current Duke PDRCE projects (Hauser et al. 2003; Noureddine et al. 2005a). By combining these two studies, we found six genes that were significantly differentially expressed between patients with PD and control samples, and that mapped to the chromosome 1p AAO linkage region (Table 14). In this study, we tested SNPs in these six genes for association with risk and AAO in PD.

Iterative association mapping. We developed a second approach, “iterative association mapping,” to identify candidate genes in a linkage region. The overall concept is to reduce the number of SNPs genotyped while maximizing the chance of discovering a significant association. SNPs are first chosen at 100 kb intervals and tested for association with traits of interest, which in this case are risk and AAO in PD. If no significant association is detected, the marker-to-marker distance is decreased by one-half each time (50 kb, 25 kb, etc.) until a significant association result is found. When a significant association is detected, additional SNPs are then tested in the surrounding region based on known linkage disequilibrium (LD) patterns, or physical iteration in the surrounding region of the associated SNP if no previous LD patterns are available.

Statistical Analyses. All SNPs were tested for Hardy-Weinberg equilibrium (HWE) and LD in the affected (one affected from each family) and unaffected groups (one unaffected from each family). An exact test implemented in the Genetic Data Analysis (GDA) program was used to test HWE, in which 3,200 permutations were performed to estimate the empirical p-value for each marker (Zaykin et al. 1995). The Graphical Overview of Linkage Disequilibrium (GOLD) package was used to calculate LD (as measured by the Pearson correlation coefficient r² and the Lewontin's standardized disequilibrium coefficient D′) between pairs of SNPs (Abecasis and Cookson 2000). Both r² and D′ range from 0 (no LD) to 1 (perfect LD). However, there is no clear definition on how to interpret intermediate LD values. Here, we chose an arbitrary cutoff by considering two markers in strong LD if r²>0.60 or D′>0.90.

AAO was treated as a quantitative trait. We used both the orthogonal model (OM) (Abecasis et al. 2000) and the Monks-Kaplan (MK) method (Monks and Kaplan 2000) implemented in the QTDT program to test the association between markers and AAO. The MK method not only provides an association signal, but also detects the direction of association, i.e., positive association for allele A is declared when the majority of allele A carriers have an AAO higher than the average AAO. In addition to nominal p-values, we also performed 10,000 permutation tests to obtain an empirical p-value for each marker based on the MK method. The global significance level was derived from permutation tests.

We performed haplotype analysis for genes with significant markers. Prior to the haplotype analysis, we identified tagging SNPs (tagSNPs) for each gene using the 1dSelect program (Carlson et al. 2004). The 1dSelect program generates groups of markers in LD on the basis of a given threshold of r². These groups are referred to as “LD-bins.” A tagSNP is then selected from each LD-bin. To perform the haplotype association analysis for AAO on the tagSNPs, we first used the FBAT-o option (Laird et al. 2000) to estimate the optimal offset of the AAO for each tagSNP. We then performed the HBAT-e option (Horvath et al. 2004) on the adjusted AAO data (subtracting AAO with the average optimal offset estimate) for testing the association between haplotypes and AAO. When the number of tagSNPs is large, the computational time is substantial and the haplotype frequencies tend to be small, which is difficult to interpret even if significant p-values are found. Therefore, we limited our haplotype computation to five tagSNPs. For genes with more than five tagSNPs, we analyzed all possible combinations of five tagSNPs.

The pedigree disequilibrium test (PDT) (Martin et al. 2000; Martin et al. 2003) was used to determine the association between markers and PD risk. Two PDT statistics were used: the PDT-sum statistic for allelic effects and the genotype-PDT for genotypic effects. We also performed haplotype analysis on the risk genes detected by PDT. The approach of selecting tagSNPs is as described above. We used HBAT-e option to test the haplotype association between a set of tagSNPs and PD.

Several criteria were used in determining the final levels of significance in the presence of multiple comparisons. First, a significance level of p≦0.05 was used for evaluating the initial set of markers with 100 kb spacing. Second, a cluster approach (described below) was used to generate a significance level for further iterations. This requires that two or more markers, which have an r² correlation <0.6, be significant within a cluster of SNPs. Finally, at least one marker in the candidate gene or region needs to meet the global significance level derived from the permutation test.

Assume a total of N markers with low LD (r²<0.6) across the region of interest and x markers located in each cluster, which leads to y cluster (y=N/x). We hypothesized that a cluster would be significant only if two markers within the cluster are significant. We can formulate the probability (α_{c}) that one out of y clusters is significant as a function of the probability of a marker being significant where α is the significance level of a marker:

$${α}_{c} = ( \begin{matrix}y \\ 1\end{matrix} ) ( \begin{matrix}x \\ 2\end{matrix} ) {{{α}^{2} ( 1 - α )}^{x - 2} [ 1 - ( \begin{matrix}x \\ 2\end{matrix} ) {{α}^{2} ( 1 - α )}^{x - 2} ]}^{y - 1} . \qquad ( 1 )$$

By restricting the significant level of a cluster to be α_{c}, we can compute the probability that a marker is significant. In other words, the probability that two markers within a cluster are significant at the level of α will result in probability α_{c} that one cluster is significant. Clearly, α decreases when the number of significant markers within a cluster decreases or when α_{c}, the significance level of a cluster, decreases. The calculation of the global significance level is described above.

The multiplex families used in this study include 167 families that were previously used in the AAO linkage study (hereafter called “the linkage data set”) (Li et al. 2002). We performed SOLAR (Almasy and Blangero 1998) PEDLOD analysis with our previous chromosome 1 peak marker (D S12134) to obtain family-specific LOD scores for the 167 families. We then stratified the linkage data set to positive and negative linkage subsets based on the family-specific LOD scores. The genes significantly associated with AAO in the overall data set were also tested for association with AAO using the MK method in the positive and negative linkage subsets. We did not use the OM approach because it requires a normal distribution for the quantitative trait of interest, which is a problem for these small, stratified data sets.

mRNA analysis for USP24. Total RNA was isolated from human midbrain tissue and reverse transcribed using poly-dT primers to generate a cDNA library. Primers to amplify fragments of the USP24 transcript were designed using the Primer3 website (Whitehead Institute for Biomedical Research; sequences available upon request). We generated several PCR products of the expected size from the cDNA library and sequenced them. Exon-intron structure of the complete USP24 transcript was deduced from genomic alignment of the overlapping RT-PCR fragments.

Identification of the linkage subsets of families. The SOLAR PEDLOD analysis of D1 S2134 identified 83 families with positive LOD scores (i.e., with positive linkage) and 84 with negative LOD scores (i.e., negative linkage) from the linkage data set (Li et al. 2002). Throughout this study, we performed association analyses with the overall PD data set as well as in these two stratified linkage subsets.

Genomic convergence. We identified two differentially expressed genes from a previous microarray study (Hauser et al. 2005) and four from a SAGE study (Noureddine et al. 2005b) that mapped to our chromosome 1p AAO linkage region (Table 14). We generated an LD pattern of these six genes (pairwise r² values) (Table 18) by analysis of SNPs (Table 17) in each of these six genes using the PD multiplex data set.

The exclusion of a gene as a candidate from an association study is not always straightforward. The degree of confidence in which one excludes a gene from association is based on the depth of the search. One measure is at the level of LD defined by the current HapMap data. Because we began genotyping our data set prior to the availability of the HapMap dataset, and because we genotyped as many SNPs with as wide of a variety of frequencies as possible from what was available in public (NCBI) and private (Applied Biosystems) databases, some of our markers are not in the HapMap data set. To evaluate whether we have sufficiently covered each gene, we compared our SNP coverage of each gene to the current HapMap data. The number of LD-bins identified on the basis of HapMap SNPs with a minor allele frequency (MAF) greater than 10% is as follows: one LD-bin for ATP6VOB, UQCRH, and C1orf8; two for TTC4; three for RNF11; and 12 for PPAP2B. Overall, our SNPs included the HapMap tagSNPs in all genes except RNF11 and PPAP2B, we missed one HapMap tagSNP in RNF11 and covered only two HapMap tagSNPs (of seven SNPs genotyped) in PPAP2B.

None of these genes show significant association with PD risk and only SNP 193 in C1orf8 was significant for association with AAO in PD. The association of SNP 193 was not verified in the positive linkage subset.

ELAVL4. The embryonic-lethal, abnormal vision, Drosophila-like 4 gene (ELAVL4) encodes for a neuron-specific RNA-binding protein. This gene was studied as a biological candidate marker through an ongoing project in the Duke PDRCE (Antic and Keene 1997). Two polymorphisms (SNPs 136 and 143) were previously found to be significantly associated with AAO in PD (Noureddine et al. 2005b). However, these markers were not found to reach significant p values in the positive linkage subset in this study.

Iterative association mapping and linkage disequilibrium. The initial association map consisted of 200 SNPs (one SNP genotyped, on average, every 100 kb) in the genomic region “one LOD score down” from the peak (40.4-59.2 Mb on NCBI build 34). With additional genotyping in the regions of interest, the average SNP density in our final association map was one marker every 66 kb, with a total of 284 SNPs genotyped. The MAFs of the SNPs varied from 0.03 and 0.50 (median and average=0.29). All but 20 SNPs (7%) were in HWE in both the affected and unaffected samples at a p=0.05 level (Table 17). The genotype distributions of these 20 SNPs were re-examined by a technician in the laboratory and tested for HWE again. The results remained the same. Considering a 5% random chance of obtaining markers not in HWE, the 7% frequency detected in our project is within a reasonable range. Furthermore, it is important to note that the MK and PDT tests do not require HWE.

The pairwise LD (as measured by the Pearson correlation coefficient r², and Lewontin's standardized disequilibrium coefficient, D1) in the group without PD, between all 264 markers in HWE was plotted. A similar LD pattern was observed in the affected group. LD is mostly restricted to intragenic areas, with no extensive LD for long stretches of DNA, or across distant loci for the majority of polymorphisms. Only SNPs with a low MAF (recent SNPs) show high levels of D′ with most neighboring SNPs.

To obtain a p value for the cluster analysis, 210 markers were identified whose r² was <0.6 for LD. Using these 210 markers and assuming 7 markers lying within each cluster, a significance level of 0.01 for each marker was derived. In addition, we obtained a global significance level of 0.001. Among the first 200 SNPs studied (100 kb map), evidence for association with AAO was found by either the OM or MK tests in the genes for translation initiation factor EIF2B3 (SNP 63, P=0.009 [OM] and P=0.0004 [MK]), the testis-specific protein kinase 2 (TESK2, SNP 76, P=0.008 [MK]), hypothetical protein FLJ14442 (SNP 117, P=0.01 [MK]), and the ubiquitin-specific protease 24 (USP24, SNP 220, P=0.004 [OM]). These markers have empirical p-values by permutation tests that are slightly lower than the nominal p-values. For example, the empirical p-value for SNP 63 in EIF2B3 was 0.0002. Evidence of association with risk for PD by use of the PD multiplex data set was found only in the human immunodeficiency virus type 1 enhancer-binding protein 3 gene (HIVEP3) for SNPs 13 (P=0.008) and 19 (P=0.004). We proceeded to increase the SNP density in these genes.

TESK2 and FLJ14442. Additional SNPs (SNPs 72, 74, 75 in TESK2, and 116, 118, 120, 122, 124 in FLJ14442) were genotyped, to a final average density of one marker per 29 kb for TESK2 and one marker per 51 kb for FLJ14442. Although we detected two sets of cluster markers for AAO association, no markers were significant after correction for multiple testing, nor did they show evidence of association in the positive linkage subset.

EIF2B3. Ten additional SNPs (SNPs 57-62 and 64-67) were genotyped in the EIF2B3 gene (136 kb), leading to a final average density of one marker per 12 kb. Several markers that were close to significance in the overall data set became significantly associated with AAO in the positive linkage subset (Table 16), despite the subset being only one-third of the total sample size (83 families). Therefore, at least two clusters of markers in low LD (r²<0.6) (SNPs 59-61 and 62-64) are strongly associated with AAO in this gene. More interestingly, SNPs 62-64 are still significant after correcting for multiple testing (P<0.001).

Five tagSNPs (SNPs 59-60, 64-66) were found in EIF2B3. Haplotype analysis with these five tagSNPs using the overall PD data set produced two haplotypes significantly associated with AAO: C-C-G-T-G (haplotype frequency=17.2%, P=0.002) and A-C-A-T-G (haplotype frequency=15.2%, P=0.002) (Table 15). These two haplotypes showed p-values comparable to what we detected for SNP 64 alone (P=0.01 by OM and 0.0001 by MK).

USP24 and AK127075. In total, we genotyped 14 SNPs (SNPs 218-231) with approximately 17 kb spacing in the region from USP24 to the cDNA FKJ45132 clone BRAWH3037979 (GenBank Accession No. AK127075), a region in which seven SNPs (SNPs 220-222, 224, 227, and 230-231) are significantly associated with AAO (p<0.01). The most significant marker was SNP 227, with P-values of 0.0006 by the OM and 0.007 by the MK method.

In silico, several lines of evidence suggested that the annotated USP24 gene in NCBI build 34 (as defined by the mRNA for KIAA1057 protein (GenBank Accession No. AB028980)) may actually be a truncated version of the full-length USP24 transcript. The 5′ end of the AB028980 transcript (exons 1-11) matches the 3′ end of the AK127075 mRNA (exons 25-35), and the human THC1877380 transcript from the TIGR Human Gene Index overlaps both genes. Genscan predicts the existence of the NT_{—}032977.390 mRNA (composed of the AB028980 and AK127075 mRNAs and 12 additional exons at the 5′ end) and there is a cluster of human overlapping spliced ESTs (e.g., GenBank Accession nos. BM458550, AW853346, and CD687922) that support the existence of a longer USP24 transcript. Furthermore, the mouse AK045043 significantly overlaps with this cluster of ESTs, but has two additional distant exons at the 5′ end. The putative first exon is supported by the FirstEF program prediction, contains an ATG start codon with sequences conforming to a Kozak consensus [(A/G)CC ATG G], has a nearby CpG island, and is close to predicted promoter sequences; all of which strongly reinforce the idea that it encodes the first exon of the larger USP24 open reading frame. This gene produces a predicted mRNA of approximately 8 kb.

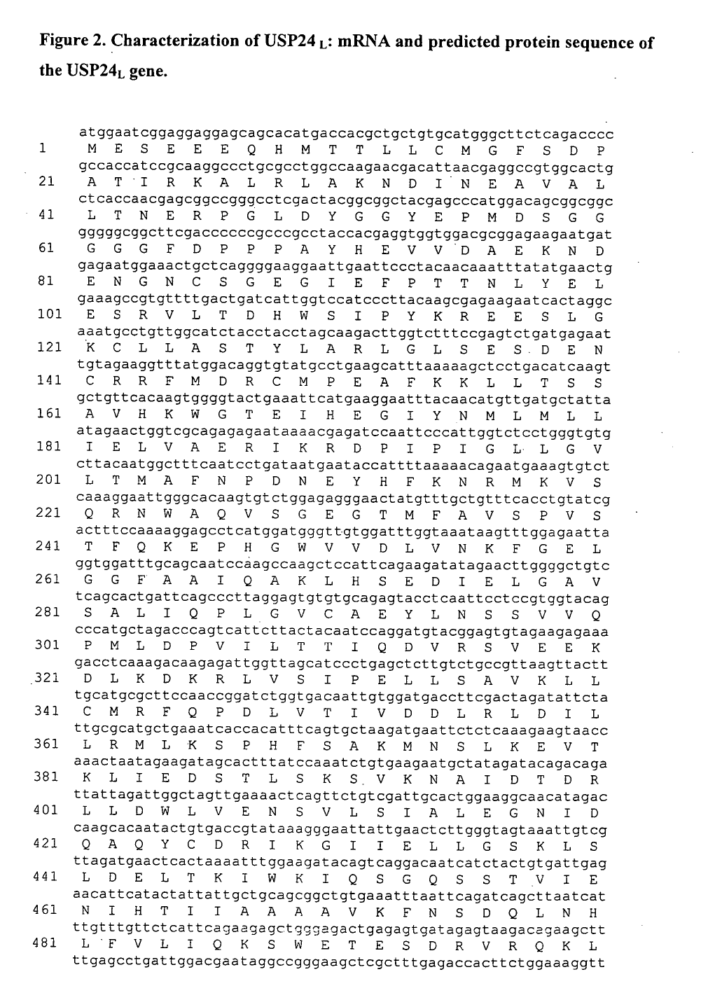

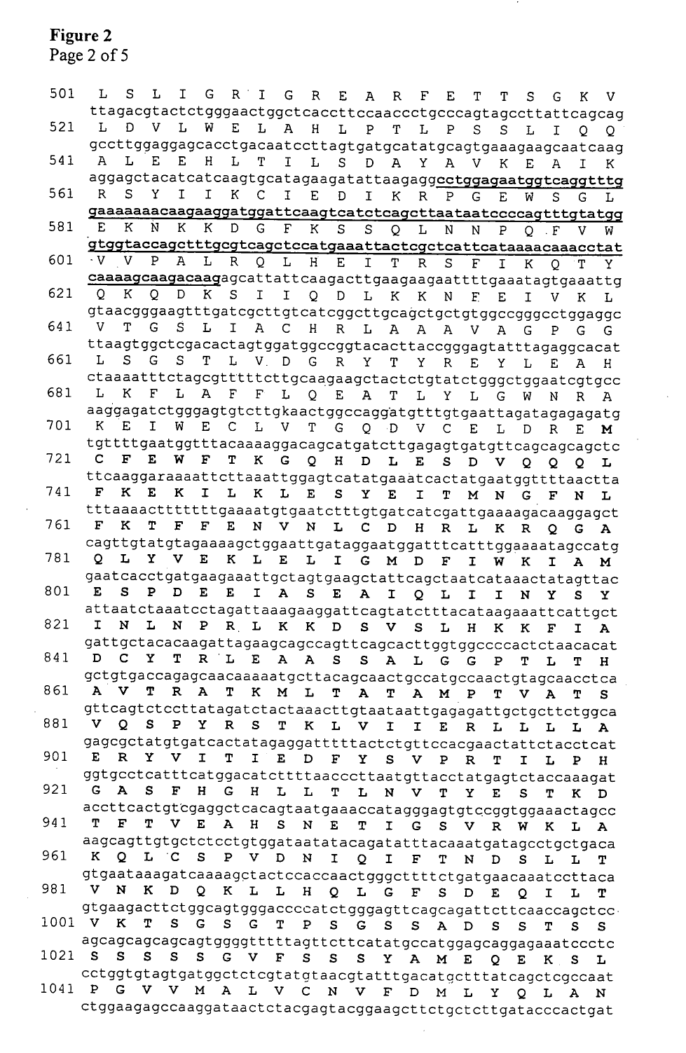

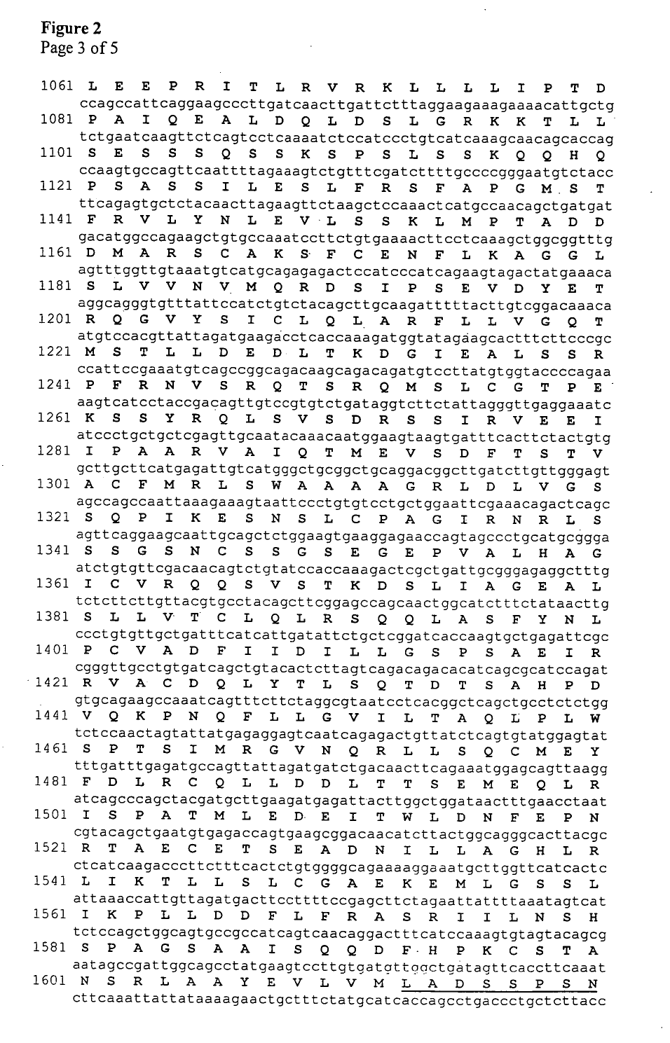

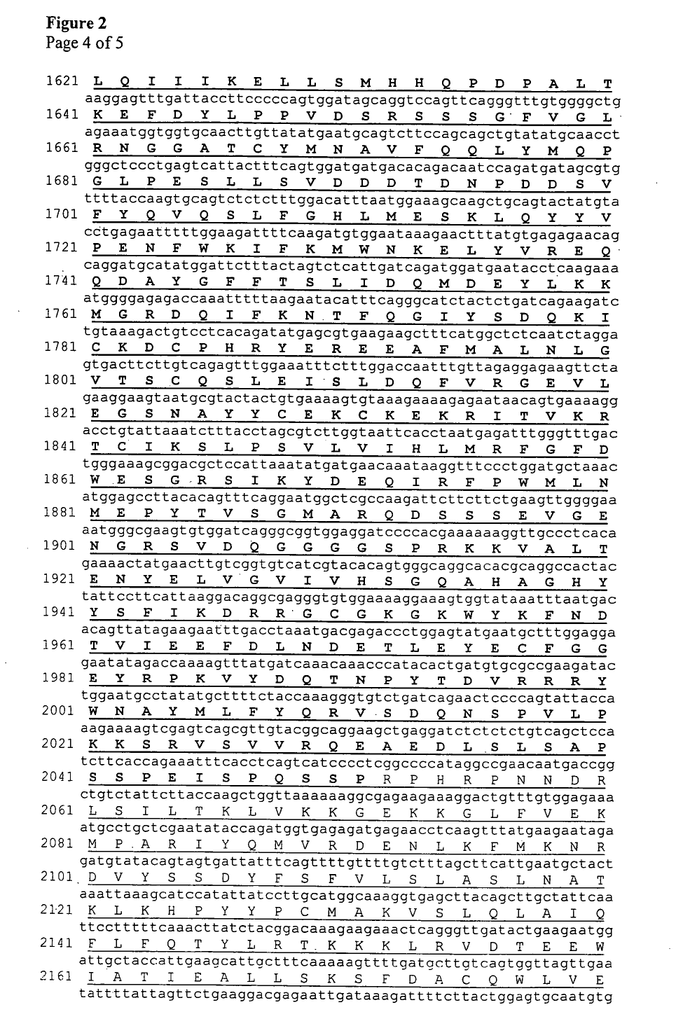

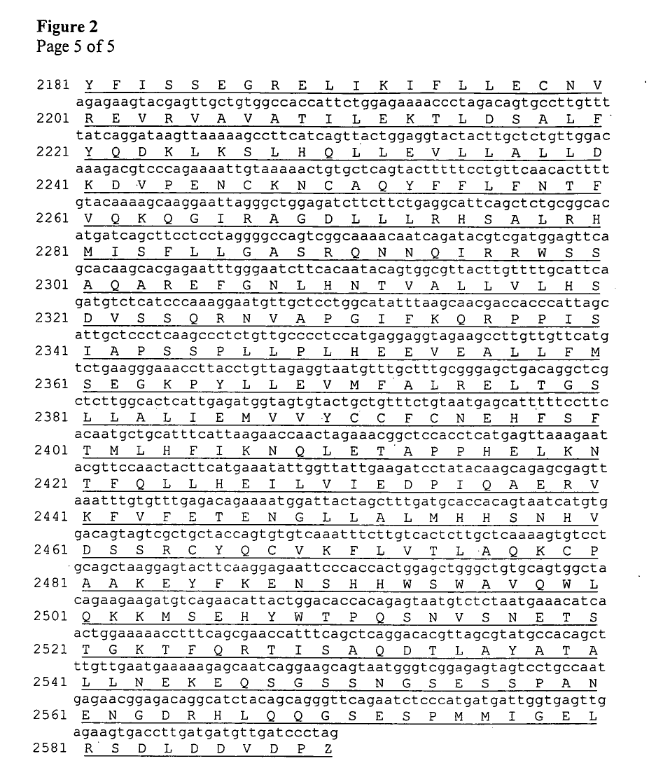

To evaluate the existence of this larger USP24 transcript, termed “USP24_{L},” we used strategically positioned primers to amplify overlapping transcript fragments from a human midbrain cDNA library. We obtained RT-PCR products of the expected sizes, and direct sequencing of these products confirmed the existence of the USP24_{L} transcript. Using the BLAT tool implemented in the University of California-Santa Cruz website, we aligned the experimentally amplified composite cDNA with the genomic sequence. The sequence of our USP24_{L} transcript (SEQ ID NO:8) carried more exons than the Genscan NT_{—}032977.390 and GNOM XM_{—}371254 predictions, some of which are supported by human or mouse ESTs. All splice junctions followed the canonical AG/GT rule. The composite cDNA is predicted to encode a protein of 2,590 amino acids (FIG. 2, SEQ ID NO:9) distributed over 69 exons and spanning over 146 kb of genomic sequence (chromosome 1: 54904635-55050704 bp). The LD block observed from SNP 216 through SNP 231, which encompasses the USP24_{L} gene and flanking regulatory sequences only, also supports the size of the USP24_{L} gene.

Since the SNPs significantly associated with AAO in this region completely span the USP24_{L} gene, and strong LD exists throughout USP24_{L} but not with neighboring genes, we concluded that the association originates from USP24_{L} itself. Three LD-bins were found in this region on the basis of the 14 SNPs genotyped (SNPs 218-231) in this study. The seven SNPs significantly associated with AAO were, in fact, originating from two LD-bins, The first LD-bin is formed by SNPs 220, 221, 224 and 230 [max. P=0.007] and the second is formed by SNPs 222, 227 and 231 [max. P=0.003]), which implies that there are two independent polymorphisms in USP24_{L} that have significant effect on AAO. Although none of the SNPs in USP24_{L} were significantly associated in either the positive or negative linkage subsets by the MK test, SNPs 221, 224, and 230 were close to significant (0.05<P<0.06) in the positive linkage subset (Table 16).

Three tagSNPs (SNPs 218, 219, and 227) were identified in USP24. Two haplotypes, C-T-T (62.6%, P=0.003) and C-T-C (19.9%, P=0.026), were found to be significantly associated with AAO (Table 15). Overall, these haplotypes in USP24 did not provide any more information on the association with AAO than SNP 227 alone.

HIVEP3. A total of nine markers in this gene were genotyped at a final average density of one marker for every 45 kb. The new SNPs failed to reveal any further significant association with risk for developing PD. However, SNP 12 was close to significant in both the allelic (P=0.058) and genotypic (P=0.057) association tests, and SNP 18 (P=0.059) was close to significant in the PDT test since it is in relatively high LD with SNP 19 (r²=0.75 in the unaffected group). To test for association of SNPs 13 and 19 in a second independent data set, we genotyped these two markers in the PD singleton data set. We did not find evidence of association of these SNPs in the singleton data set alone. However, both markers showed stronger significant association in the combined multiplex and singleton data set (P=0.006 [SNP 13] and P=0.002 [SNP 19]) than in the multiplex data set. Clearly, some singleton families also contribute to the association of these two markers.

We identified eight tagSNPs (SNPs 13-17, 19-21) in HIVEP3. Haplotype analyses based on five tagSNPs revealed the best results by use of tagSNPs 13, 15, 17, 19, and 21, in which a rare A_G_T_G_C haplotype (frequency: 2.1%) was significantly associated with risk for PD (P=0.003) (Table 15). HIVEP3 is a relatively large gene (408 kb) and very low levels of LD were observed among the SNPs genotyped. The lack of LD between SNPs 13 and 19 (r²=0 and D′=0.02) provides two independent lines of evidence for the involvement of this gene in controlling risk for developing PD.

In this study, we present a systematic approach termed “iterative association mapping” to identify susceptibility genes and genetic modifiers in a linkage region. This methodology has the advantage of being unbiased by any pre-conceived ideas about the pathogenic mechanisms of a disease (as in candidate gene studies). In addition, our analysis strategies include single locus association tests in the overall, positive, and negative linkage subsets, as well as haplotype association analysis based on tagSNPs in the overall data set.

Because a large number of SNPs was tested in this study, we wished to correct for multiple testing while maintaining an appropriate threshold to screen for potential areas of association, without eliminating any potential candidates. The Bonferroni correction is too conservative and would become exclusionary at a time when we want to avoid missing any potential associations. One can prioritize genes based on the order of p-values or use the global significance level derived from the permutation test, but either method may exclude too many potential leads and therefore these options do not fit the purpose of the first few iterations. Therefore, we added an intermediate criterion for analysis, as we considered the presence of multiple significant markers in low LD within a regional cluster to be more important than sporadic results across the region. The concept of this method is relatively straightforward: if multiple comparisons lead to significant SNPs only by chance, then these false positive SNPs (if we assume for the moment that all SNPs in high LD are the same measure) should be randomly distributed across the physical region to be tested. That is, there is no reason for them to be clustered physically together if they are just significant only due to chance. Thus, we are seeking two SNPs with a defined level of significance that lie within a small physical region, and have a correlation that is low enough (r²<0.6) that the significant associations of each individual marker with AAO are not likely the result of measuring the same chance event. This approach allows us to lower the significance level, which is more stringent than the conventional approach using a nominal significance level, and take into account the locations of the significant markers.

The EIF2B3 gene ranks as the most significant AAO gene in this region. Two clusters of markers in this gene were significantly associated with AAO in the overall set and positive linkage subsets. We also detected two clusters of markers in USP24 that are significantly associated with AAO at both significance levels of p=0.01 and p=0.001. However, the association evidence was not as strong as EIF2B3 due to less significant findings in the positive linkage subset. We therefore would consider USP24 to be the second most significant AAO gene in the region for further follow-up. Finally, HIVEP3 is the only gene found in this region that is associated with risk for developing PD.

The finding of multiple associated genes under the peak was unexpected. If one assumes that not all of the statistically significant genes found here are biologically important in PD, is there a way to prioritize them for further study? Conceptually, as linkage analysis localized the initial peak (Li et al. 2002), the associations we identified should be “responsible” for the linkage. Thus, we identified those families contributing to the chromosome 1 linkage localization and examined this subset for association. However, by reducing the sample size to one third (only 83 families had positive LOD scores at marker D1S2134), one would expect that the P-values of the associated SNPs would become less significant on the basis of power alone. But in reducing the sample size, we also expect to render our sample more homogeneous and therefore to increase the significance in the true susceptibility polymorphisms. The most significant polymorphism in EIF2B3 remained equally significant despite the sample size loss, while two polymorphisms in EIF2B3 (SNPs 59 and 61) that were close to significant in the overall data set became more significant in the positive linkage subset. This implicates EIF2B3 in controlling the AAO of Parkinson disease. The ability to subdivide the data on the basis of linkage also demonstrates one of the additional strengths of family-based association data.

EIF2B3 is the γ subunit of the heteropentamer eIF2B (α, β, γ, δ, and ε subunits). The translation initiation factor eIF2B catalyzes the exchange of guanine nucleotides on the initiation factor, eI2F, which itself mediates the binding of the initiator Met-tRNA to the 40S ribosomal subunit during translation initiation. EI2FB is important because it regulates global rates of protein synthesis, particularly when the cell is under mild cellular stress. Protein synthesis is generally decreased during periods of cellular stress in order to lower the amount of detrimental unfolded and damaged proteins that can be toxic to the cell (van der Knaap et al. 2002). Interestingly, eIF2B causes vanishing white matter disease (VWM [MIM 603896]), an autosomal recessive disorder characterized by cerebellar ataxia, spasticity, inconstant optic atrophy and a relatively mild mental decline. The early-onset of this disease reflects the hypothetical maximal expression levels of eIF2B −β, −γ, −δ, and −ε during embryonic development and lower levels with aging (Inamura et al. 2003). It is well known that mild head trauma or fever is highly correlated with rapid clinical decline in these patients. Van der Knapp et al. suggested that this clinical deterioration is due to the failure of eIF2B in the critical role of regulating protein synthesis under mild cellular stress. Furthermore, the observed phenotypic variation in patients with identical eIF2B mutations suggests that genetic polymorphisms may influence the effect of the mutation (van der Knaap et al. 2002). Thus, the biological activity of this gene fits well with the current ideas of cellular stress having a major role in PD.

USP24, the second most significant AAO gene, is a member of the family of ubiquitin-specific proteases (USPs) that remove polyubiquitin from target proteins, rescuing them from degradation by the proteasome. Wherein genes involved in the proteolytic pathway and aggregation of proteins (Parkin, α-synuclein) contribute to PD pathology, USP24 appears also to be an excellent biological candidate gene for controlling AAO in Parkinson disease. We identified several polymorphisms in USP24 significantly associated with AAO, one of which (SNP 220) is non-synonymous (alanine to valine change). The effect of this polymorphism on protein function is not currently known.

Unlike EIF2B3 and USP24, HIVEP3 was found to be associated with the risk of developing PD. The HIVEP3 protein is a member of the HIVEP (human immunodeficiency virus [HIV] enhancer-binding protein) family that encodes large zinc finger proteins and regulates transcription via the κB enhancer motif (Allen et al. 2002). This motif is an important element controlling the transcription of viral genes and many cellular genes that are involved in immunity, cell cycle regulation, and inflammation. As we reported previously, the GSTO1 (glutathione S-transferase omega 1) gene is associated with AAO of PD (Li et al. 2003), and also possibly plays a role in inflammation during the pathogenesis of PD, because of its involvement in the post-translational modification of the inflammatory cytokine interleukin-1β (Laliberte et al. 2003). The mouse homolog of HIVEP3, the kappa recognition component (KRC), participates in the signal transduction pathway leading from the tumor necrosis factor (TNF) receptor to gene activation, and may play a critical role in inflammatory and apoptotic responses (Oukka et al. 2002). Patients with HIV have been reported to have decreased levels of dopamine (DA), but normal levels of other neurotransmitters, suggesting selective and profound loss of DA neurons (Lopez et al. 1999).

### References for Example 4

Abecasis et al. (2000) A general test of association for quantitative traits in nuclear families. Am J Hum Genet 66:279-292
Abecasis and Cookson (2000) GOLD—graphical overview of linkage disequilibrium. BioInformatics 16:182-183
Allen et al. (2002) The kappa B transcriptional enhancer motif and signal sequences of V(D)J recombination are targets for the zinc finger protein HIVEP3/KRC: a site selection amplification binding study. BMC Immunol 3: 10
Almasy and Blangero (1998) Multipoint quantitative-trait linkage analysis in general pedigrees. Am J Hum Genet 62:1198-1211
Antic & Keene (1997) Embryonic lethal abnormal visual RNA-binding proteins involved in growth, differentiation, and posttranscriptional gene expression. Am J Hum Genet 61:273
Blomqvist et al. (2004) Sequence variation in the proximity of IDE may impact age at onset of both Parkinson disease and Alzheimer disease. Neurogenetics 5:115-119
Carlson et al. (2004) Selecting a maximally informative set of single-nucleotide polymorphisms for association analyses using linkage disequilibrium. Am J Hum Genet 74:106-120
Destefano et al. (2002) PARK3 influences age at onset in Parkinson disease: a genome scan in the GenePD study. Am J Hum Genet 70:1089-1095
Fahn et al. (1987) Unified Parkinson Disease rating scale. In Fahn et al. (eds.) Recent Developments in Parkinson Disease. Florham Park, N.J.: MacMillan Health Care Information
Hauser et al. (2005)Expression Profiling of Substantia Nigra in Parkinson, PSP, and FTDP-17. Arch Neurol 62:917-921
Hauser et al. (2003) Genomic convergence: identifying candidate genes for Parkinson disease by combining serial analysis of gene expression and genetic linkage. Hum Mol Genet 12:671-677
Haynes et al. (1995) PEDIGENE: A comprehensive data management system to facilitate efficient and rapid disease gene mapping. Am J Hum Genet 57:A193
Hicks et al. (2002) A susceptibility gene for late-onset idiopathic Parkinson disease. Ann Neurol 52:549-555
Horvath et al. (2004) Family-based tests for associating haplotypes with general phenotype data: application to asthma genetics. Genet Epidemiol 26:61-69
Hubble et al. (1999) Parkinson Disease: Clinical features in sibships. Neurology 52:A13
Inamura et al. (2003) Developmental changes of eukaryotic initiation factor 2B subunits in rat hippocampus. Neurosci Lett 346:117-119
Karamohamed et al. (2003) A haplotype at the PARK3 locus influences onset age for Parkinson disease: the GenePD study. Neurology 61:1557-1561
Kitada et al. (1998) Mutations in the parkin gene cause autosomal recessive juvenile parkinsonism. Nature 392:605-608
Kolsch et al. (2004) Polymorphisms in glutathione S-transferase omega-1 and AD, vascular dementia, and stroke. Neurology 63:2255-2260
Laird et al. (2000) Implementing a unified approach to family-based tests of association. Genet Epidemiol 19 Suppl 1:S36-S42
Laliberte et al. (2003) Glutathione s-transferase omega 1-1 is a target of cytokine release inhibitory drugs and may be responsible for their effect on interleukin-1beta posttranslational processing. J Biol Chem 278:16567-16578
Leroy et al. (1998) Deletions in the Parkin gene and genetic heterogeneity in a Greek family with early onset Parkinson disease. Hum Genet 103:424-427
Li et al. (2004) Apolipoprotein E controls the risk and age at onset of Parkinson Disease. Neurology 62:2005-2009
Li et al (2003) Glutathione S-transferase omega-1 modifies age-at-onset of Alzheimer disease and Parkinson disease. Hum Mol Genet 12:3259-3267
Li et al (2002) Age at onset in two common neurodegenerative diseases is genetically controlled. Am J Hum Genet 70:985-993
Lopez et al. (1999) Dopamine systems in human immunodeficiency virus-associated dementia. Neuropsychiatry Neuropsychol Behav Neurol 12:184-192
Martin et al. (2003) Genotype-based association test for general pedigrees: the genotype-PDT. Genet Epidemiol 25:203-213
Martin et al. (2000) A test for linkage and association in general pedigrees: the pedigree disequilibrium test. Am J Hum Genet 67:146-154
Monks and Kaplan (2000) Removing the sampling restrictions from family-based tests of association for a quantitative-trait locus. Am J Hum Genet 66:576-592
Noureddine et al. Genomic Convergence to identify candidate genes for Parkinson disease: SAGE analysis of the substantia nigra. Mov Disord online publication Jun. 17, 2005
Noureddine et al. Association between the neuron-specific RNA-binding protein ELAVL4 and Parkinson disease. Hum Genet April, 2005
Oukka et al. (2002) A mammalian homolog of Drosophila schnurri, KRC, regulates TNF receptor-driven responses and interacts with TRAF2. Mol Cell 9:121-131
Paisan-Ruiz et al. (2004) Cloning of the gene containing mutations that cause PARK8-linked Parkinson disease. Neuron 44:595-600
Polymeropoulos et al. (1997) Mutation in the alpha-synuclein gene identified in families with Parkinson disease. Science 276:2045-2047
Scott et al (2001) Complete genomic screen in Parkinson disease: evidence for multiple genes. JAMA 286:2239-2244
Valente et al. (2004) Hereditary early-onset Parkinson disease caused by mutations in PINK1. Science 304:1158-1160
van der Knaap et al. (2002) Mutations in each of the five subunits of translation initiation factor eIF2B can cause leukoencephalopathy with vanishing white matter. Ann Neurol 51:264-270
Zaykin et al. (1995) Exact tests for association between alleles at arbitrary numbers of loci. Genetica 96:169-178

### Example 5

### Mitochondrial Polymorphisms Associated with Parkinson Disease

Mitochondrial (mt) impairment, particularly within complex I of the electron transport system, has been implicated in the pathogenesis of Parkinson disease (PD). More than half of mitochondrially encoded polypeptides form part of the NADH dehydrogenase (ND) complex I enzyme. To test the hypothesis that mtDNA variation contributes to PD expression, we genotyped ten single nucleotide polymorphisms (SNPs) that define the European mtDNA haplogroups (H, I, J, K, T, U, V, W and X) in 609 Caucasian PD patients and 340 unaffected Caucasian controls. Overall, individuals classified as haplogroup J [odds ratio (OR)=0.55;95%, confidence interval (CI)=0.34-0.91;p=0.02] or K (OR=0.52;95% CI=0.30-0.90;p=0.02) demonstrated a significant decrease in risk of PD versus individuals carrying the most common haplogroup, H. Furthermore, a specific SNP that defines these two haplogroups, 10398G, is strongly associated with this protective effect (OR=0.53;95% CI=0.39-0.73;p=0.0001). SNP 10398G causes a non-conservative amino acid change from threonine to alanine within ND3 of complex I. Stratification by sex revealed that this decrease in risk appeared stronger in females (OR=0.43;95% CI=0.27-0.71;p=0.0009). Additionally, SNP 9055A of ATP6 also demonstrated a protective effect within females (OR=0.45; 95% CI=0.22-0.93;p=0.03).

Subjects. A total of 609 unrelated Caucasian PD cases were included in this study. Cases were ascertained through the Duke Center for Human Genetics (DCHG) Morris K. Udall Parkinson's Disease Center of Excellence and from the DCHG/GlaxoSmithKline Parkinson's Disease Genetics Collaboration. The 340 Caucasian controls were collected from spouses of Alzheimer disease patients ascertained through the Joseph and Kathleen Bryan Alzheimer's Disease Research Center. Controls had no significant signs of cognitive or neurological impairment when enrolled in the study. Mean age-at-onset (AAO) in affected individuals in the sample is 62±12 years (mean±SD). AAO is self reported by the PD patient and defined as the age at which the affected individual first noticed one of the cardinal signs of PD. PD patient mean age-at-examination (AAE) is 66±12 years while control mean AAE is 69±9 years. AAE was defined as the age at which study personnel clinically examined the affected or unaffected participant. The overall sample consists of 57% males and 43% females. The PD case group is composed of 63% males and 37% females while the control group consists of 44% males and 56% females. Written consent was obtained from all participants in agreement with protocols approved by the institutional review board at each contributing center. A board-certified neurologist specializing in movement disorders or physician assistant experienced in neurological disorders examined individuals following rigorous clinical criteria for diagnosis of PD. All PD patients had at least two principal signs of PD (resting tremor, bradykinesia, rigidity) and no clinical features of any other parkinsonian syndromes.

Classification of Haplogroups. Ten SNPs within coding genes and the control region were chosen for genotyping (Torroni et al. (1996)). SNPs within restriction fragment length polymorphism (RFLP) sites were identified so that the allelic discrimination method Taqman® could be employed (Table 19). By comparing the complete, revised Cambridge genomic sequence (Andrews et al. 1999) with the Japanese (Anderson et al. 1981), Swedish (Arnason et al. 1996) and African (Horai et al. 1995) reference sequence genomes, we were able to identify the nucleotide change within each restriction site. (Mitochondrial reference sequences: Cambridge (#NC001807), revised Cambridge (#J01415), Japanese (#AB055387), Swedish (#X93334) and African (#D38112)).

SNP Genotyping. Genomic DNA was isolated from whole blood samples by the DCHG DNA banking Core using Puregene (Gentra Systems, Minneapolis, MN). High-throughput genotyping was established using the 5′ nuclease allelic discrimination Taqman® assay in a 384 well format on the ABI Prism® 7900HT Sequence Detection System (Applied Biosystems, Foster City, Calif.). In each chamber of the 384-well sample plates, 20 ng of DNA was distributed using a Hydra HTS Workstation microdispensing system (Robbins Scientific, Sunnyvale, Calif.). Probes and primers for each SNP were designed using ABI Prism® Primer Express software Version 2.0 (Applied Biosystems, Foster City, Calif.). All probes designed with a black-hole quencher reporter were generated by Integrated DNA Technologies, Inc. (Coralville, Iowa) and all minor groove binding (MGB) Taqman probes were manufactured by Applied Biosystems (Foster City, Calif.).

To each well, 5 μl of master mix (0.2 U/μl Taqman®V Universal PCR Master Mix; 0.9 ng/μl of each forward and reverse primer; and 0.2 ng/μl of each probe) was dispensed by a MultiProbe2 204DT (Packard Instruments, Shelton, Conn.). The amplification reaction was conducted on an ABI Dual 384-well GeneAmp® PCR System 9700 utilizing the following program: 50° C. for 2 minutes; 95° C. for 10 minutes; 95° C. for 15 seconds and 62° C. for 1 minute, repeated for 40 cycles; and held at 4° C. upon cycling completion. Data were generated on an ABI Prism® 7900HT Sequence Detection System (SDS) and analyzed using the associated SDS version 2.0 software.

The few samples falling outside SNP clusters were sequenced for genotyping. Sequencing primers were designed using the Vector NTI Suite 6 software package (InforMax, Inc., Bethesda, Md.) and Primer3 website. DNA sequencing was conducted on an ABI Prism® 3100 Genetic Analyzer (Applied Biosystems, Foster City, Calif.). Sequencing analysis was performed using the ABI Prism® Sequencing Analysis Software version 3.7 and Sequencher® software version 4.0.5. In addition to the positive controls, four negative controls were also assayed per plate. For quality control, samples for 24 individuals were duplicated per each 384-well plate. Technicians performing the SNP genotyping were blinded to the duplications. Additionally, two DNA samples from the Centre d'Etude du Polymorphisme Humain (CEPH) were sequenced for each SNP, plated eight times per plate, and also used as blind internal controls. All quality control samples were compared in the Duke Center for Human Genetics Data Coordinating Center. Data were stored and managed by the PEDIGENE® system (Haynes et al. 1995).

Statistical Analysis. All statistical analyses were performed using SAS software release 8.1 (SAS Institute Inc., Cary, N.C.). Statistical significance was declared at α=0.05. A t-test was conducted to test for differences in AAE between cases and controls, with a significant difference found (p-value=0.0001). To assess differences in distribution of sex between cases and controls we used a chi-square test, and found a significant difference in distribution (p-value=0.0001). Therefore, to adjust for potential confounding, we used AAE and sex as covariates in the analyses. We performed unconditional logistic regression to generate odds ratios with their associated 95% confidence intervals to assess odds of carrying each mitochondrial SNP in PD cases compared to controls. In addition, we used unconditional logistic regression to simultaneously assess odds of PD cases carrying specific haplogroups. Since haplogroup carrier status was a categorical independent variable with more than two categories, there are multiple ways to assign the reference group: each haplogroup can be compared against a common haplogroup or each haplogroup can be compared against all other haplogroups pooled into one group. An advantage of using a common haplogroup as the reference is that it is more homogeneous than pooling different haplogroups and means that each haplogroup is compared to the same reference group for consistency. We performed the analysis using both approaches for comparison. Firstly, H was chosen as a reference group since it is found at the highest frequency (40-50%) among European populations. We also tested for association of a specific haplogroup, for example K, relative to all other haplogroups by pooling frequencies of non-K. This is conceptually the same as the binary SNP allele comparison. P-values reported for SNPs and haplogroups are based on the Wald chi-square statistic for the particular SNP or haplogroup, and are not adjusted for multiple testing.

All nine major European haplogroups were observed in our sample and did not differ significantly from a previous study of a similar North American control population (Torroni et al. 1994). (Table 20) In addition, a nearly identical percentage of individuals (8.2% in controls and 8.5% in PD cases) did not fit into these nine pre-defined haplogroups and were classified as “others.” This group most likely consists of rare European haplogroups (R, Z, etc.) or the historical admixture known to exist in the North American Caucasian population (Richards et al. 2000; Finnila et al. 2000). Therefore, comparison of overall population haplogroups suggests that the control population was well matched to our PD cases and supports an absence of significant substructure.

Evaluation of genotyping results revealed 100% match of all duplications using the Taqman method. Though heteroplasmy was not specifically tested, we did not observe the occurrence of multiple mtDNA copies (wild-type and mutant) in any individual sequenced (N=125).

Both haplogroup J (OR=0.55; 95% CI, 0.34 to 0.91; p=0.02) and haplogroup K (OR=0.52; 95% CI, 0.31 to 0.90; p=0.02) were found less frequently, relative to the common haplogroup H, in PD cases compared to controls (Table 21). A similar finding (p=0.03) was revealed when each haplogroup was analyzed by comparing it relative to all other haplogroups pooled together. In comparing what made these two haplogroups (J and K) unique from the other haplogroups tested, one SNP located at position 10398 was identified. We therefore tested this SNP independently and found that the 10398G allele frequency between PD patients and controls was highly significant (OR=0.53; 95% CI, 0.39 to 0.73; p=0.0001). The 10398G allele causes a non-conservative amino acid change from Threonine (hydrophilic) to Alanine (hydrophobic) within the NADH dehydrogenase 3 gene (ND3) which is a subunit of complex I. Further stratification of the data set by sex revealed that the 10398G effect appeared to be stronger in females (OR=0.43; 95% CI, 0.27 to 0.71; p=0.0009) compared to males (OR=0.62; 95% CI, 0.41 to 0.97; p=0.04). Moreover, this analysis showed that SNP 9055A, found within the ATP6 gene, has a mild protective effect in only females when compared to males (OR=0.46; 95% CI, 0.22 to 0.91; p=0.03) (Table 21). Additionally, we found that SNP allele 13708A, located within ND5, is protective in the ≧70 group (OR=0.27; 95% CI, 0.09 to 0.77; p=0.01).

Both associated polymorphisms (10398G, 13708A) cause nonconservative amino acid changes from Threonine (Thr) to Alanine (Ala) within ND3 and Ala to Thr within ND5. These subunits are two of the seven mitochondrially-encoded peptides making up the 43 enzymatic subunits of complex I.

Our data demonstrated that the apparent protective effect of the 10398G allele was stronger in the female set (p=0.0009) compared to males (p=0.04). Furthermore, SNP allele 9055A, which partly defines haplogroup K, was found to decrease PD risk only in females. These findings are interesting given the results from multiple clinical studies that male incidence of PD is higher than that of females (ranging from 1.5-2.5 males: 1.0 females) (Tanner and Goldman 1996; Swerdlow et al. 2001).

In addition, we have shown that stratification by gender revealed that males classified as haplogroup U showed an increased risk of developing PD (OR=2.2, p=0.03) when compared to all other males classified as haplogroup H.

Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference into this application in order to more fully describe the state of the art to which this invention pertains.

### References for Example 5

Anderson et al. (1981) Sequence and organization of the human mitochondrial genome. Nature 290:457-465
Andrews et al. (1999) Reanalysis and revision of the Cambridge reference sequence for human mitochondrial DNA. Nat Genet 23:147
Ardlie et al. (2002) Testing for population subdivision and association in four case-control studies. Am J Hum Genet 71:304-311
Arnason et al. (1996) Comparison between the complete mitochondrial DNA sequences of Homo and the common chimpanzee based on nonchimeric sequences. J Mol Evol 42:145-152
Betarbet et al. (2002) Animal models of Parkinson's disease. Bioessays 24:308-318
Betarbet et al. (2000) Chronic systemic pesticide exposure reproduces features of Parkinson's disease. Nat Neurosci 3:1301-1306
Brown et al. (2002) The role of mtDNA background in disease expression: a new primary LHON mutation associated with Western Eurasian haplogroup J. Hum Genet 110:130-138
Cassarino et al. (1997) Elevated reactive oxygen species and antioxidant enzyme activities in animal and cellular models of Parkinson's disease. Biochim Biophys Acta 1362:77-86
De Benedictis et al. (2000) Does a retrograde response in human aging and longevity exist? Exp Gerontol 35:795-801
De Benedictis et al. (1999) Mitochondrial DNA inherited variants are associated with successful aging and longevity in humans. FASEB J 13:1532-1536
Finnila et al. (2000) Phylogenetic network of the mtDNA haplogroup U in Northern Finland based on sequence analysis of the complete coding region by conformation-sensitive gel electrophoresis. Am J Hum Genet 66:1017-1026
Greenamyre et al. (1999) Mitochondrial dysfunction in Parkinson's disease. Biochem Soc Symp 66:85-97
Greenamyre et al. (2001) Complex I and Parkinson's disease. IUBMB Life 52:135-141
Gu et al. (1998) Mitochondrial DNA Transmission of the Mitochondrial defect in Parkinson's Disease. Ann Neurol 44: 177-186
Haynes et al. (1995) PEDIGENE: A comprehensive data management system to facilitate efficient and rapid disease gene mapping. Am J Hum Genet 57:A193
Herrnstadt et al. (2002) Reduced-median-network analysis of complete mitochondrial DNA coding-region sequences for the major African, Asian, and European haplogroups. Am J Hum Genet 70:1152-1171
Horai et al. (1995) Recent African origin of modern humans revealed by complete sequences of hominoid mitochondrial DNAs. Proc Natl Acad Sci USA 92:532-536
Jenner & Olanow (1998) Understanding cell death in Parkinson's disease. Ann Neurol 44:S72-S84
Muthane et al. (2001) Hunting genes in Parkinson's disease from the roots. Med Hypotheses 57:51-55
Orth & Schapira (2002) Mitochondrial involvement in Parkinson's disease. Neurochem Int 40:533-541
Parker et al. (1989) Abnormalities of the electron transport chain in idiopathic Parkinson's disease. Ann Neurol 26:719-723
Richards et al. (2000) Tracing European founder lineages in the Near Eastern mtDNA pool. Am J Hum Genet 67:1251-1276
Schapira et al. (1990) Mitochondrial complex I deficiency in Parkinson's disease. J Neurochem 54: 823-827
Schapira et al. (1989) Mitochondrial complex I deficiency in Parkinson's disease. Lancet 1:1269
Sherer et al. (2002) An in vitro model of Parkinson's disease: linking mitochondrial impairment to altered alpha-synuclein metabolism and oxidative damage. J Neurosci 22:7006-7015
Simon et al. (2000) Mitochondrial DNA mutations in complex I and tRNA genes in Parkinson's disease. Neurol 54:703-709
Swerdlow et al. (2001) Gender ratio differences between Parkinson's disease patients and their affected relatives. Parkinsonism Relat Disord 7:129-133
Swerdlow et al. (1996) Origin and functional consequences of the complex I defect in Parkinson's disease. Ann Neurology 40:663-671
Tanner & Goldman (1996) Epidemiology of Parkinson's disease. Neurol Clin 14:317-335
Torroni et al. (1996) Classification of European mtDNAs from an analysis of three European populations. Genetics 144:1835-1850
Torroni et al. (1994) mtDNA and the Origin of Caucasians: Identification of Ancient Caucasian-specific Haplogroups, One of Which is Prone to a Recurrent Somatic Duplication in the D-Loop Region. Am J Hum Genet 55:760-776
Torroni & Wallace (1994) Mitochondrial DNA variation in human populations and implications for detection of mitochondrial DNA mutations of pathological significance. J Bioenerg Biomembr 26:261-271
Trimmer et al. (2000) Abnormal mitochondrial morphology in sporadic Parkinson's and Alzheimer's disease cybrid cell lines. Exp Neurol 162:37-50
Veech et al. (2000) Disrupted mitochondrial electron transport function increases expression of anti-apoptotic bcl-2 and bcl-X(L) proteins in SH-SY5Y neuroblastoma and in Parkinson disease cybrid cells through oxidative stress. J Neurosci Res 61: 693-700

Wallace et al. (1999) Mitochondrial DNA variation in human evolution and disease. Gene 238:211-230

TABLE 1
Results of single locus and genotype association analyses

| | PDTsum | genoPDT |
|---|---|---|
| Overall | | |
| 8P0217 | 0.1616 | 0.4077 |
| rs1989756 | 0.3942 | 0.4355 |
| rs1989754 | 0.0006 | 0.0056 |
| rs1721100 | 0.0196 | 0.0713 |
| 8p0215 | 0.0008 | 0.0004 |
| Hx+ | | |
| 8P0217 | 0.2902 | 0.5984 |
| rs1989756 | 0.1218 | 0.2111 |
| rs1989754 | 0.0033 | 0.0249 |
| rs1721100 | 0.2058 | 0.3344 |
| 8p0215 | 0.0047 | 0.0042 |

TABLE 2
Haplotype analysis of FGF20
Estimated haplotypes in the overall dataset

| | SNPs genotyped | | | | | | | | |
|---|---|---|---|---|---|---|---|---|---|
| | 8p0217 | rs1989756 | rs1989754 | rs1721100 | 8p0215 | #Families | Frequency | Z | p-value |
| h1 | 1 | 2 | 1 | 2 | 1 | 228 | 0.42 | −3.318 | 0.0009 |
| h2 | 2 | 2 | 2 | 2 | 1 | 205 | 0.21 | 0.294 | ns |
| h3 | 2 | 2 | 2 | 1 | 1 | 179 | 0.19 | 0.691 | ns |
| h4 | 1 | 2 | 2 | 1 | 2 | 80 | 0.08 | 3.587 | 0.0003 |
| h5 | 2 | 1 | 2 | 2 | 1 | 89 | 0.06 | 0.465 | ns |
| h6 | 1 | 2 | 2 | 2 | 1 | 11 | 0.008 | −0.488 | ns |
| h7 | 2 | 1 | 2 | 1 | 1 | 25 | 0.005 | −0.254 | ns |
| | | | | | | | | Global test | 0.003 |

7 degrees of freedom
ns = not significant

TABLE 3
Multilocus genotype PDTsum analysis

Genotype

| A | B | Z | p-value |
|---|---|---|---|
| 1, 1 | 1, 1 | −2.480 | 0.013 |
| 1, 1 | 1, 2 | 0.000 | 1.000 |
| 1, 2 | 1, 1 | −0.912 | 0.362 |
| 1, 2 | 1, 2 | 0.000 | 0.946 |
| 2, 2 | 1, 1 | 0.697 | 0.486 |
| 2, 2 | 1, 2 | 2.785 | 0.005 |
| 2, 2 | 2, 2 | 0.810 | 0.423 |

A rs1989754
B 8p0215

TABLE 4
Linkage disequilibrium test of FGF 20 SNPs
LD test - R2

| | RS1989756 | RS1989754 | RS1721100 | 8p0215 |
|---|---|---|---|---|
| Affected | | | | |
| 8P0217 | 0.086 | 0.652 | 0.045 | 0.097 |
| RS1989756 | | 0.058 | 0.018 | 0.009 |
| RS1989754 | | | 0.268 | 0.073 |
| RS1721100 | | | | 0.259 |
| Unaffected | | | | |
| 8P0217 | 0.081 | 0.677 | 0.069 | 0.09 |
| RS1989756 | | 0.058 | 0.018 | 0.004 |
| RS1989754 | | | 0.267 | 0.058 |
| RS1721100 | | | | 0.245 |

LD test - D prime

| | 8P0217 | RS1989756 | RS1989754 | RS1721100 | 8p0215 |
|---|---|---|---|---|---|
| Affected | | | | | |
| 8P0217 | | 1 | 0.986 | 0.315 | 0.968 |
| RS1989756 | | | 1 | 0.724 | 1 |
| RS1989754 | | | | 0.943 | 0.961 |
| RS1721100 | | | | | 1 |
| Unaffected | | | | | |
| 8P0217 | | 1 | 0.979 | 0.399 | 1 |
| RS1989756 | | | 1 | 0.75 | 0.717 |
| RS1989754 | | | | 0.94 | 0.873 |
| RS1721100 | | | | | 1 |

TABLE 5
Chromosome regions (genes) linked to Parkinson disease.

| Chromosome | Genes |
|---|---|
| 5 | Synphilin and the ubiquitin conjugating enzyme (UBE2B) |
| 6 | Parkin |
| 8 | NAT1 and NAT2 |
| 9 | Two proteasome subunits (Z and S5) PSMB7, PSMD5; |
| | Torsin A (DYT1) or Torsin B |
| 17 | Ubiquitin B (UBB) and Tau (MAPT) |

TABLE 6
Genomic regions generating LOD scores greater than 1
in the PD genomic screen.

| | | | |
|---|---|---|---|
| | 40 cM Interval | | |
| | on Marshfield | | |
| | 1998 Sex- | | Strata in which |
| | Averaged | Marker boundaries | interval has |
| Peak Marker | Map | for 40 cM Interval | LOD > 1 |

Chromosome 2

| | | | |
|---|---|---|---|
| D2S1329 | 0-35 | D2S2982-D2S1240 | Early onset |
| D2S405 | 26-68 | D2S1400-D2S2291 | Early onset |
| D2S410 | 105-145 | D2S2161-D2S1334 | Early onset |
| D2S434 | 192-232 | D2S161-D2S2297 | Dopa |
| | | | responsive* |

Chromosome 3

| | | | |
|---|---|---|---|
| D3S1768 | 41-81 | D3S1554-D3S3631 | Non-dopa |
| | | | responsive |
| D3S2460 | 114-154 | D3S1251-D3S3546 | Non-dopa |
| | | | responsive |

Chromosome 5

| | | | |
|---|---|---|---|
| D5S2848 | 20-60 | D5S2064-D5S1968 | Overall**, late |
| | | | onset**, Dopa |
| | | | responsive** |
| D5S186 | 119-159 | D5S2027-D5S1499 | Overall, early |
| | | | onset**, late |
| | | | onset**, dopa |
| | | | responsive** |
| D5S1480 | 139-179 | D5S816-D5S1960 | Non-dopa |
| | | | responsive |

Chromosome 6

| | | | |
|---|---|---|---|
| D6S305 | 146-186 | D6S1703-D6S1027 | Early onset |
| D6S503 | 164-193 | D6S1581-D6S2522 | Non-dopa |
| | | | responsive |

Chromosome 8

| | | | |
|---|---|---|---|
| D8S520 | 0-40 | D8S504-D8S258 | Overall, |
| | | | late-onset, |
| | | | dopa |
| | | | responsive |

Chromosome 9

| | | | |
|---|---|---|---|
| D9S301 | 46-86 | D9S259-D9S776 | Non dopa |
| | | | responsive |
| D9S2157 | 126-166 | D9S1811-D9S2168 | Overall, late |
| | | | onset, |
| | | | non-dopa |
| | | | responsive |

Chromosome 10

| | | | |
|---|---|---|---|
| D10S1432 | 73-113 | D10S122-D10S1755 | Early onset** |

Chromosome 11

| | | | |
|---|---|---|---|
| D11S4131 | 118-147 | D11S4132-D11S4112 | Early onset |

Chromosome 12

| | | | |
|---|---|---|---|
| D12S398 | 48-88 | D12S1042-D12S64 | Early onset** |

Chromosome 14

| | | | |
|---|---|---|---|
| D1421426 | 105-138 | D14S291-D14S544 | Overall**, late |
| | | | onset**, dopa |
| | | | responsive |

Chromosome 17

| | | | |
|---|---|---|---|
| D17S921 | 16-56 | D17S1854-D17S1293 | Overall, early |
| | | | onset |
| D17S1293 | 36-76 | D17S921-D17S669 | Late-onset, |
| | | | dopa |
| | | | responsive |

Chromosome 21

| | | | |
|---|---|---|---|
| D21S1437 | 0-33 | D21S1911-D21S1895 | Late onset, |
| | | | dopa |
| | | | responsive |

Chromosome 22

| | | | |
|---|---|---|---|
| D22S685 | 12-52 | D22S425-D22S928 | Late onset**, |
| | | | dopa |
| | | | responsive**, |
| | | | non-dopa |
| | | | responsive** |

Chromosome X

| | | | |
|---|---|---|---|
| GATA165B12 | 113-153# | DXS6796-DXS1205 | Overall**, |
| | | | late-onset**, |
| | | | dopa |
| | | | responsive** |
| DXYS154 | 164-184# | DXS9908-X | Late onset**, |
| | | telomere | dopa |
| | | | responsive** |

*= Multipoint LOD > 1 only
**= Single point LOD > 1 only
#= female map distances

TABLE 7
Parkin mutations detected.

| | Amino Acid | # | # | Mean | | |
|---|---|---|---|---|---|---|
| Nucleotide Change | Change | individuals | families | AO | Range | Ref. |
| Homozygous | Stop | 5 | 2 | 38.0 | 19-53 | |
| 438-477 del 40 bp | | | | | | |
| 438-477 del 40 bp + 1390 | Stop + Gly430Asp | 2 | 1 | 25.5 | 22-29 | Gly > Asp¹ |
| G > A | | | | | | |
| 438-477 del 40 bp | Stop | 9 | 4 | 35.0 | 21-57 | |
| only | | | | | | |
| All 438-477 del 40 bp | Stop | 16 | 7 | 34.8 | 19-57 | |
| 924 C > T + 1412 | Arg275Trp + Pro > Leu | 2 | 1 | 45.0 | 38-52 | Arg > Trp² |
| C > T | | | | | | |
| 924 C > T + 859 | Arg275Trp + Cys > Tyr + Pro > Leu | 2 | 1 | 24.0 | 21-27 | |
| G > A + 1412 | | | | | | |
| C > T | | | | | | |
| 924 C > T only | Arg275Trp | 4 | 4 | 54.0 | 39-71 | |
| | only | | | | | |
| All 924 C > T | All | 8 | 6 | 44.3 | 21-71 | |
| | Arg275Trp | | | | | |
| Homozygous | Gln34/Stop37 | 2 | 1 | 25.5 | 19-32 | Del |
| 202-203 del AG | | | | | | AG² |
| 199 G > A + G > T | Arg > Gln + G > T | 2 | 1 | 16.5 | 12-21 | |
| exon 9 + 4³ | in intron | | | | | |
| 346 C > A | Ala > Glu | 1 | 1 | 62.0 | 62 | |
| 885 G > A | Asp > Asn | 1 | 1 | 52.0 | 52 | |
| All Mutations | | 28 | 17 | 39.6 | 12-71 | |

1) Lucking et al., New England Journal of Medicine 342: 1560-7 (2000)
2) Abbas et al., Human Molecular Genetics 8: 567-74 (1999)
3) Refers to the position 4 base pairs pat the end of exon 9, e.g., in the intron.

TABLE 8
Composition of the data set: Number of Affected Relative Pairs*

| | |
|---|---|
| Mean number of sampled affected members per family | 2.3 ± 0.6 |
| | (range: 2-6) |
| Mean number of sampled affected relative pairs per family | 1.5 ± 1.4 |
| | (range 1-15) |
| Number of sampled affected sibpairs | 185 |
| Number of sampled affected avuncular pairs | 19 |
| Number of sampled affected cousin pairs | 51 |
| Number of sampled affected parent-child pairs | 5 |
| Total number of affected relative pairs | 260 |

*all possible affected relative pairs counted

TABLE 9
Regions generating multipoint LOD* greater than 1.

| | | peak | Two-point | | Multipoint | |
|---|---|---|---|---|---|---|
| Chromosome Set | | marker | MLOD | location | Peak LOD* | location |
| 3q | NLDR | D3S2460 | 1.62 | 135 | 1.54 | 134 |
| 5q | ALL | D5S816 | 2.39 | 139 | 1.5 | 139 |
| | NLDR | D5S820 | 1.47 | 160 | 1.04 | 153 |
| 6q | EOPD | D6S305 | 5.07 | 166 | 5.47 | 166 |
| 8p | ALL | D8S520 | 2.01 | 21 | 2.22 | 27 |
| | LOPD | D8S520 | 1.96 | 21 | 1.92 | 27 |
| 9q | NLDR | D9S301 | 1.52 | 66 | 1.01 | 66 |
| 9q | ALL | D9S2157 | 1.59 | 147 | 1.47 | 147 |
| | LOPD | D9S2157 | 1.36 | 147 | 1.4 | 145 |
| | NLDR | D9S2157 | 0.98 | 147 | 2.59 | 140 |
| 11q | EOPD | D11S4131 | 1.22 | 139 | 1.53 | 139 |
| 17q | ALL | D17S921 | 1.92 | 36 | 2.02 | 56 |
| | LOPD | D17S1293 | 2.05 | 56 | 2.31 | 56 |
| | NLDR | D17S1843 | 2.52 | 41 | 1.26 | 36 |

EOPD = early-onset PD;
LOPD = late-onset PD;
NLDR = non-levodopa-responsive PD

TABLE 10
PCR primers and OLA probes for SNPs used in association analyses.

| | | |
|---|---|---|
| SNP | PCR primer (SEQ ID NO:) | OLA probe (SEQ ID NO:) |

| | | | | | |
|---|---|---|---|---|---|
| 3 | IVS3+9A>G | forward | gggctgctttctggcatatg (14) | Allele 1 G | 5′-Cy3-aggaaccacaggtgagggt |
| | | | | | g (16) |
| | | reverse | cctcacttctgtcacaggtc (15) | Allele 2 A | 5′-Cy3-agaaggaaccacaggtgaggg |
| | | | | | ta (17) |
| | | | | common | 5′-Pho-agccccagagacccccaggcag |
| | | | | | tc (18) |
| 9i | c1632A >G | forward | ccacccgggagcccaagaaggtgcc (19) | Allele 1 G | 5′-Fam-gggagcccaagaaggtggc |
| | | | | | g (21) |
| | Ala544Ala | reverse | ctggtgcttcaggttctcagtg (20) | Allele 2 A | 5′-Fam-cccgggagcccaagaaggtg |
| | | | | | gca (22) |
| | | | | common | 5′-Pho-gtggtccgtactccacccaagtcg |
| | | | | | ccgtcttccgc (23) |
| 9ii | c1716T >C | forward | cgagtcctggcttcactcc (24) | Allele 1 C | 5′-Cy3-ccatgccagacctgaagaa |
| | | | | | c (26) |
| | Asn572Asn | reverse | cttccaggcacagccatacc (25) | Allele 2 T | 5′-Cy3-tgcccatgccagacctgaaga |
| | | | | | at (27) |
| | | | | common | 5′-Pho-gtcaagtccaagatcggctccact |
| | | | | | gaga (28) |
| 9iii | c1761G >A | forward | cgagtcctggcttcactcc (29) | Allele 1 A | 5′-Fam-agaacctgaagcaccagcc |
| | | | | | a (31) |
| | Pro587Pro | reverse | cttccaggcacagccatacc (30) | Allele 2 G | 5′-Fam-ctgagaacctgaagcaccagcc |
| | | | | | g (32) |
| | | | | common | 5′-Pho-ggaggcgggaaggtgagagtggct |
| | | | | | gg (33) |
| 11 | IVS11 +34G >A | forward | gctcattctctctcctcctc (34) | Allele 1 A | 5′-Cy3-ggtgagggttgggacggga |
| | | | | | a (36) |
| | | reverse | ccaggactcctccaccccatgcagc (35) | Allele 2 G | 5′-Cy3-gaaggtgagggttgggacggga |
| | | | | | g (37) |
| | | | | common | 5′-Pho-ggtgcagggggtggaggagtcct |
| | | | | | ggtgaggctggaac (38) |

TABLE 11
P-values for PDT and Transmit single-locus tests.

| | MLEs for Allele | | |
|---|---|---|---|
| SNP | Frequencies¹ | PDT² | Transmit² |
| 3 | 0.794 | 0.062 | 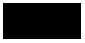 |
| 9i | 0.793 | 0.076 | 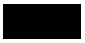 |
| 9ii | 0.790 | 0.113 | 0.106 |
| 9iii | 0.955 | 0.638 | 0.866 |
| 11 | 0.793 | 0.055 | 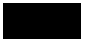 |

¹For positively associated allele
²P-values from chi-squared distribution
Note:
P-values ≦ 0.05 are highlighted.

TABLE 12
P-values for Transmit tests for five-locus SNP haplotypes.

| Haplotype for | |
|---|---|
| 3/9i/9ii/9iii/11 | P-values |
| 11121 | 0.007 |
| 22212 | 0.863 |
| 22222 | 0.009 |
| Global Test | 0.024 |

Note:
Individual haplotype tests are compared to a chi-square distribution with 1 df. Global test is compared to chi-square distribution with 2df.

TABLE 13
P-values for single-locus and 5-locus haplotype
Transmit tests in stratified data sets.

| | Family-history | Family-history | Early | Late |
|---|---|---|---|---|
| | positive | negative | onset | onset |
| SNPs | (N = 181) | (N = 54) | (N = 39) | (N = 196) |
| 3 | 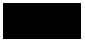 | 0.957 | 0.076 | 0.076 |
| 9i | 0.055 | 0.645 | 0.682 | 0.059 |
| 9ii | 0.128 | 0.585 | 0.534 | 0.149 |
| 9iii | 0.707 | 0.170 | 0.076 | 0.816 |
| 11 | 0.055 | 0.524 | 0.199 | 0.095 |
| Haplotype for 3/9i/9ii/9iii/11 | 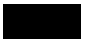 | 0.479 | 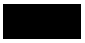 | 0.093 |

Note
P-values < 0.05 are highlighted. N is the number of families in the stratum.

TABLE 14
Genes differentially expressed in PD cases versus controls in
microarray and serial analysis of gene expression (SAGE)
experiments that map to the chromosome 1p AAO linkage peak.

| | | | PD vs | |
|---|---|---|---|---|
| | | | Control | |
| | Gene | UniGene ID | fold | |
| Gene name | symbol | or clone_id | change | P-value* |
| Ubiquinol-cytochrome | UQCRH | 202233_s_at | −1.4 | 0.0244 |
| c reductase | | | | |
| hinge protein⁺ | | | | |
| ATPase, | ATP6V0B | 200078_s_at | −1.3 | 0.0356 |
| H+ transporting, | | | | |
| lysosomal | | | | |
| 21 kDa, V0 subunit c⁺ | | | | |
| Ring finger | RNF11 | Hs. 96334 | −4.1 | <0.0001 |
| protein 11 | | | | |
| Chromosome 1 open | C1orf8 | Hs. 416495 | 3.6 | 0.0006 |
| reading frame 8 | | | | |
| Tetratricopeptide | TTC4 | Hs. 412482 | −12.3 | 0.0149 |
| repeat domain 4 | | | | |
| Phosphatidic acid | PPAP2B | Hs. 432840 | −6.2 | 0.0359 |
| phosphatase type 2B | | | | |

(2005)⁺ and Noureddine et al. (2005a).
*These P-values were not corrected for multiple testing and were obtained from Hauser et al.

TABLE 15
Summary of haplotypes showing significant association with AAO in the overall
PD data set. The keys to SNP numbers are listed in Table 17.

| Gene | Marker 1 | Marker 2 | Marker 3 | Marker 4 | Marker 5 | Frequency | P-value |
|---|---|---|---|---|---|---|---|
| C1orf8 | SNP 192_G | SNP 193_A | SNP 194_C | | | 66.4% | 0.004 |
| | SNP 192_G | SNP 193_T | SNP 194_C | | | 29% | 0.009 |
| TESK2 | SNP 72_C | SNP 75_A | SNP 76_A | | | 40.6% | 0.012 |
| FLJ14442 | SNP 117_T | SNP 118_A | SNP 119_C | SNP 121_A | SNP 123_A | 7.5% | 0.037 |
| | SNP 117_G | SNP 118_C | SNP 119_C | SNP 121_A | SNP 123_A | 6.7% | 0.018 |
| EIF2B3 | SNP 59_C | SNP 60_C | SNP 64_G | SNP 65_T | SNP 66_G | 17.2% | 0.002 |
| | SNP 59_A | SNP 60_C | SNP 64_A | SNP 65_T | SNP 66_G | 15.2% | 0.002 |
| USP24 | SNP 218_C | SNP 219_T | SNP 227_T | | | 62.6% | 0.003 |
| | SNP 218_C | SNP 219_T | SNP 227_C | | | 19.9% | 0.026 |
| HIVEP3 | SNP 13_A | SNP 15_G | SNP 17_T | SNP 19_G | SNP 21_C | 2.1% | 0.003 |

TABLE 16
Summary of P-values from orthogonal model (OM) and Monks-Kaplan
(MK) method for markers in EIF2B3 and USP24 in the overall,
positive linkage, and negative linkage data sets.

| | | | | | Positive | Negative |
|---|---|---|---|---|---|---|
| | | | | | linkage | linkage |
| | | | Overall data set | | subset | subset |
| | SNP | | (N = 267) | | (N = 83)* | (N = 84)* |
| Gene | ID | Probe name | OM | MK** | MK | MK |
| EIF2B3 | 57 | rs12733586 | 1.000 | 0.325 | 0.714 | 0.460 |
| | 58 | rs12139143 | 0.584 | 0.288 | 0.820 | 0.496 |
| | 59 | rs263977 | 0.109 | 0.039 | 0.005 | 0.138 |
| | 60 | rs263978 | 0.663 | 0.590 | 0.160 | 0.850 |
| | 61 | rs263965 | 0.099 | 0.041 | 0.003 | 0.210 |
| | 62 | rs1022814 | 0.012 | 0.001 | 0.001 | 0.034 |
| | 63 | rs12405721 | 0.018 | 0.0005 | 0.001 | 0.045 |
| | 64 | rs546354 | 0.01 | 0.0004 | 0.0003 | 0.096 |
| | 65 | rs566063 | 0.663 | 0.078 | 0.655 | 0.250 |
| | 66 | rs364482 | 0.842 | 0.598 | 0.767 | 0.890 |
| | 67 | rs489676 | 0.055 | 0.046 | 0.013 | 0.160 |
| USP24 | 218 | rs13312 | 0.122 | 0.274 | 0.068 | 0.483 |
| | 219 | rs1043671 | 0.791 | 0.850 | N/A | N/A |
| | 220 | rs487230 | 0.004 | 0.039 | 0.115 | 0.655 |
| | 221 | rs683880 | 0.006 | 0.049 | 0.057 | 0.245 |
| | 222 | rs667353 | 0.002 | 0.061 | 0.273 | 0.811 |
| | 223 | rs615652 | 0.232 | 0.757 | 0.177 | 0.743 |
| | 224 | rs594226 | 0.007 | 0.094 | 0.052 | 0.889 |
| | 225 | rs567734 | 0.124 | 0.221 | 0.071 | 0.714 |
| | 226 | rs625219 | 0.249 | 0.626 | 0.113 | 0.736 |
| | 227 | rs1165226 | 0.001 | 0.007 | 0.440 | 0.662 |
| | 228 | rs1024305 | 0.116 | 0.196 | 0.071 | 0.714 |
| | 229 | rs287234 | 0.632 | 0.648 | N/A | N/A |
| | 230 | rs287235 | 0.001 | 0.004 | 0.058 | 0.166 |
| | 231 | rs2047422 | 0.003 | 0.007 | 0.648 | 0.487 |

*In total, 167 out of 267 families were included in the previous AAO genomic screen study (Li et al. 2002). The positive linkage subset includes families with a positive LOD score at D1S2134 and the negative linkage subset includes those with a negative LOD score.
**P-values ≦0.01 are highlighted in bold and 0.01<P-values ≦0.05 are in italic.
Markers that are not informative for the MK test are listed as N/A.

TABLE 17

| Single nucleotide polymorphisms (SNPs) analyzed: The SNP identification | | | | | | | | | |
|---|---|---|---|---|---|---|---|---|---|
| numbers used throughout Example 4 are indicated in the first column of this table. | | | | | | | | | |
| The second column gives the official dbSNP name (if available). SNPs that do not | | | | | | | | | |
| have an rs number can be located by the primers and probes sequence or Applied | | | | | | | | | |
| Biosystems assay ID number (fourth column), or by their NCBI Build 34 genomic | | | | | | | | | |
| position (fifth column). Finally, the minor allele frequencies (MAF) in the | | | | | | | | | |
| control sample and the Hardy-Weinberg equilibrium (HWE) p-values in the normal | | | | | | | | | |
| and affected groups are shown in the last three columns. | | | | | | | | | |
| SNP | | | ABI Assay ID or | | Celera | NCBI Build | MAF | HWE | |
| ID | Probe name | Gene | Primers and Probes | | Location | 34 Location | Control | Normal | Affected |
| 1 | rs11208299 | FLJ21144 | C_25755461_10 | | 39263124 | 40394025 | 36.2 | 0.207 | 0.694 |
| 2 | rs570671 | RIM 3 | C_11868741_1_{—} | | 39373520 | 40504421 | 20.0 | 0.078 | 0.495 |
| 3 | rs6702983 | NFYC | C_{———}36079_10 | | 39483570 | 40614551 | 22.5 | 0.315 | 0.406 |
| 4 | rs729589 | KCNQ4 | GGTGGGTCCTCTGTGCAA | (SEQ ID NO:39) | 39583332 | 40714313 | 47.2 | 0.558 | 0.387 |
| | | | GGCTGATTATTTTAGGACCAGGAAACA | (SEQ ID NO:40) | | | | | |
| | | | VIC-CTATTGACTCATAtGCCTTG-NFQ | (SEQ ID NO:41) | | | | | |
| | | | FAM-TATTGACTCATAcGCCTTG-NFQ | (SEQ ID NO:42) | | | | | |
| 5 | rs7523029 | CTPS | C_{——}376232_10 | | 39732787 | 40863153 | 29.9 | 0.498 | 0.879 |
| 6 | rs3738369 | FLJ23878 | C_{———}42611_1_{—} | | 39769329 | 40899702 | 11.0 | 0.459 | 0.273 |
| 7 | rs2024859 | SCMH1 | C_11740023_1_{—} | | 39845243 | 40975579 | 11.2 | 0.712 | 0.247 |
| 8 | rs6656085 | SCMH1 | C_{——}1484416_10 | | 39924291 | 41054621 | 20.5 | 0.298 | 0.862 |
| 9 | rs4131949 | | C_{———}374440_10 | | 40021599 | 41151931 | 46.7 | 0.473 | 0.712 |
| 10 | rs7547654 | | C_{———}264011_10 | | 40114286 | 41244655 | 43.1 | 0.381 | 0.227 |
| 11 | rs2095289 | | C_{——}1774080_10 | | 40217855 | 41347902 | 42.6 | 0.760 | 0.628 |
| 12 | rs747459 | | C_{——}3056556_10 | | 40245933 | 41375975 | 29.9 | 0.081 | 0.268 |
| 13 | rs648178 | HIVEP3 | C_{——}1654040_10 | | 40284466 | 41415457 | 23.1 | 0.842 | 0.183 |
| 14 | rs1007221 | HIVEP3 | C_{——}1654075_10 | | 40322097 | 41453065 | 10.8 | 1.000 | 0.328 |
| 15 | rs2038978 | HIVEP3 | C_{——}3160228_10 | | 40377052 | 41508013 | 47.2 | 0.013 | 1.000 |
| 16 | rs10493099 | HIVEP3 | TGCCTGACCCTTACTGCAATTT | (SEQ ID NO:43) | 40476147 | 41600499 | 2.8 | 1.000 | 1.000 |
| | | | CCTATGCACCTACCTACGTCTCTT | (SEQ ID NO:44) | | | | | |
| | | | VIC-TTTTAAAAGCTCATAAGCTAGAAC-NFQ | (SEQ ID NO:45) | | | | | |
| | | | FAM-AAGCTCATAGGCTAGAAC-NFQ | (SEQ ID NO:46) | | | | | |
| 17 | rs1039997 | HIVEP3 | C_{——}1471920_10 | | 40513403 | 41644400 | 35.0 | 0.275 | 0.663 |
| 18 | rs616366 | HIVEP3 | C_{——}3177926_10 | | 40560078 | 41691075 | 38.1 | 1.000 | 0.789 |
| 19 | rs661225 | HIVEP3 | C_{——}1778763_10 | | 40592456 | 41723459 | 37.6 | 0.543 | 0.045 |
| 20 | rs710229 | HIVEP3 | C_{——}8374669_10 | | 40619538 | 41750542 | 20.2 | 0.644 | 1.000 |
| 21 | rs7554964 | HIVEP3 | C_{——}1974841_10 | | 40660515 | 41791523 | 44.4 | 0.575 | 1.000 |
| 22 | rs11210568 | | C_{——}2038148_10 | | 40796745 | 41927746 | 42.3 | 0.561 | 0.903 |
| 23 | rs1047047 | GUCA2B | C_11291674_10 | | 40901426 | 42032433 | 16.1 | 0.061 | 0.810 |
| 24 | rs16829212 | KIAA1041 | C_{——}1488855_10 | | 40938817 | 42070113 | 45.2 | 0.776 | 0.176 |
| 25 | rs1125792 | KIAA1041 | C_{——}8374853_10 | | 41031314 | 42162627 | 24.6 | 0.704 | 0.158 |
| 26 | rs12036838 | | C_11864308_10 | | 41119493 | 42250829 | 45.0 | 0.653 | 0.178 |
| 27 | rs2275116 | | C_{——}1805838_1_{—} | | 41210917 | 42342273 | 34.5 | 0.515 | 0.599 |
| 28 | rs12038786 | BX640642 | C_25642179_10 | | 41303751 | 42435104 | 34.2 | 0.621 | 0.604 |
| 29 | rs3768026 | PPIH | C_{——}1689877_10 | | 41408693 | 42540060 | 34.6 | 1.000 | 0.059 |
| 30 | rs3738505 | | C_{——}1689837_1_{—} | | 41514809 | 42646171 | 24.1 | 0.158 | 0.616 |
| 31 | rs9960 | LOC51058 | C_{——}8375036_10 | | 41599779 | 42731087 | 20.7 | 0.415 | 0.837 |
| 32 | rs3738515 | | C_{——}1166211_1_{—} | | 41708713 | 42839915 | 49.5 | 0.043 | 0.415 |
| 33 | rs515781 | | GCCTCCCAGGAACAGGAT | (SEQ ID NO:47) | 41817105 | 42948307 | 9.8 | 0.687 | 1.000 |
| | | | CGCTGAGAAGGTGCCATTTT | (SEQ ID NO:48) | | | | | |
| | | | VIC-CCATAGAATTCACGGGACAA-NFQ | (SEQ ID NO:49) | | | | | |
| | | | FAM-CCATAGAATTCATGGGACAA-NFQ | (SEQ ID NO:50) | | | | | |
| 34 | rs674684 | | C_{——}3138229_10 | | 41905257 | 43036439 | 39.2 | 1.000 | 0.237 |
| 35 | rs3862227 | | C_{——}3138279_10 | | 42003093 | 43134288 | 39.5 | 0.450 | 1.000 |
| 36 | rs839763 | CDC20 | C_{——}8375554_10 | | 42107798 | 43238938 | 37.4 | 0.538 | 0.158 |
| 37 | rs839761 | LOC149469 | C_{——}1799825_10 | | 42146009 | 43277151 | 41.1 | 0.190 | 0.393 |
| 38 | rs6954 | KIAA0467 | C_{——}1799810_1_{—} | | 42198839 | 43329936 | 40.9 | 1.000 | 0.358 |
| 39 | rs2782641 | PTPRF | C_{——}1799763_10 | | 42295238 | 43426649 | 38.6 | 0.448 | 0.612 |
| 40 | rs613976 | JMJD2A | C_{———}992847_10 | | 42401831 | 43533291 | 48.0 | 0.316 | 0.807 |
| 41 | rs11579637 | SIAT6 | C_{———}336312_10 | | 42505719 | 43637180 | 42.0 | 0.253 | 0.384 |
| 42 | rs3011225 | SIAT6 | C_{——}2982431_10 | | 42601223 | 43732667 | 21.6 | 1.000 | 0.464 |
| 43 | rs1990150 | IPO13 | C_11733857_10 | | 42697660 | 43827421 | 14.3 | 1.000 | 0.794 |
| 44 | rs2286241 | ATP6V0B | C_11291594_10 | | | 43854063 | 6.6 | 0.112 | 0.599 |
| 45 | rs2286243 | ATP6V0B | C_25474361_10 | | | 43854827 | 6.9 | 0.119 | 1.000 |
| 46 | rs12410334 | ATP6V0B | C_{——}1252855_10 | | 42726060 | 43855815 | 16.7 | 1.000 | 0.671 |
| 47 | rs2428953 | ATP6V0B | GTGCTTGACTGAGTTGATTCTTAGTG | (SEQ ID NO:51) | 42726998 | 43856753 | 10.6 | 0.416 | 0.519 |
| | | | GGACAGACAACCACAGAGTTACG | (SEQ ID NO:52) | | | | | |
| | | | VIC-ACTTCTCTCCGTCTGTC-NFQ | (SEQ ID NO:53) | | | | | |
| | | | FAM-ACTTCTCTCCATCTGTC-NFQ | (SEQ ID NO:54) | | | | | |
| 48 | rs1766967 | SLC6A9 | C_{——}8375736_1_{—} | | 42759125 | 43888880 | 6.6 | 0.192 | 0.595 |
| 49 | rs1408919 | | C_{——}3144502_10_{—} | | 42854654 | 43984422 | 33.3 | 0.411 | 0.529 |
| 50 | rs709267 | DMAP1 | C_{——}2515512_10_{—} | | 42964806 | 44094777 | 39.5 | 1.000 | 0.428 |
| 51 | rs325143 | PRNPIP | C_{——}2558254_10_{—} | | 43057058 | 44187021 | 32.1 | 0.099 | 0.889 |
| 52 | rs3866642 | FLJ10597 | C_{——}9773842_10_{—} | | 43169118 | 44299216 | 44.7 | 0.572 | 1.000 |
| 53 | rs270724 | FLJ10597 | TTCCTTTCACCCTCATACAAACATC | (SEQ ID NO:55) | 43274474 | 44404572 | 21.7 | 0.675 | 0.171 |
| | | | GCCAACGTTCCTGCTGAATAG | (SEQ ID NO:56) | | | | | |
| | | | FAM-CTGCTCTTTTGAGACCATTCGATCCTCT-BHQ1 | (SEQ ID NO:57) | | | | | |
| | | | TET-TGCTCTTTTGAGGCCATTCGATCC-BHQ1 | (SEQ ID NO:58) | | | | | |
| 54 | rs11585508 | FLJ10597 | C_3210787_10 | | 43365235 | 44495634 | 40.4 | 0.757 | 0.466 |
| 55 | rs6683133 | FLJ22353 | C_9774292_10 | | 43416450 | 44546855 | 49.5 | 0.497 | 0.715 |
| 56 | rs12732939 | KIF2C | C_149689_10 | | 43504326 | 44634726 | 18.9 | 0.037 | 0.098 |
| 57 | rs12733586 | EIF2B3 | C_3072600_10 | | 43609971 | 44740524 | 19.2 | 0.034 | 0.051 |
| 58 | rs12139143 | EIF2B3 | C_3072605_10 | | 43632815 | 44763322 | 19.3 | 0.045 | 0.059 |
| 59 | rs263977 | EIF2B3 | AGTGTGACTTTATTGAAAACATGATGCTTTT | (SEQ ID NO:59) | 43643074 | 44773581 | 38.0 | 0.215 | 0.518 |
| | | | GCAATCCTTTGTTATATTTTACCTCTGAGAGT | (SEQ ID NO:60) | | | | | |
| | | | VIC-CCCTGTGTTATTTATG-NFQ | (SEQ ID NO:61) | | | | | |
| | | | FAM-CCCTGTGTTCTTTATG-NFQ | (SEQ ID NO:62) | | | | | |
| 60 | rs263978 | EIF2B3 | C_{——}3072613_10 | | 43645780 | 44776286 | 41.1 | 0.054 | 0.618 |
| 61 | rs263965 | EIF2B3 | C_{——}808948_10 | | 43658314 | 44788819 | 38.6 | 0.449 | 0.603 |
| 62 | rs1022814 | EIF2B3 | C_{——}8725461_10 | | 43696617 | 44827140 | 18.7 | 0.455 | 0.152 |
| 63 | rs12405721 | EIF2B3 | C_{——}3072628_10 | | 43697204 | 44827727 | 18.4 | 0.627 | 0.110 |
| 64 | rs546354 | EIF2B3 | CACCATGCCTGGCCAAAAG | (SEQ ID NO:63) | 43714435 | 44844958 | 19.6 | 0.099 | 0.324 |
| | | | CCGGTTCTCTTCCTTCAGAGG | (SEQ ID NO:64) | | | | | |
| | | | VIC-AAAGCGTAGTTAAAAGCATA-NFQ | (SEQ ID NO:65) | | | | | |
| | | | FAM-AAGCGTAGTTAAGAGCATA-NFQ | (SEQ ID NO:66) | | | | | |
| 65 | rs566063 | EIF2B3 | C_809016_10 | | 43733621 | 44864129 | 24.5 | 0.058 | 0.433 |
| 66 | rs364482 | EIF2B3 | GGGAATCATGGCAACGAGTCT | (SEQ ID NO:67) | 43734263 | 44864771 | 12.9 | 0.206 | 1.000 |
| | | | AGTCTGAGATGCGGTGAACAC | (SEQ ID NO:68) | | | | | |
| | | | VIC-AAAGCTTGGGAGGCAG-NFQ | (SEQ ID NO:69) | | | | | |
| | | | FAM-AGCTTGGAAGGCAG-NFQ | (SEQ ID NO:70) | | | | | |
| 67 | rs489676 | EIF2B3 | GGCAGAAGTCACAGCTATAACTCA | (SEQ ID NO:71 | 43735013 | 44865521 | 43.8 | 0.674 | 0.896 |
| | | (5′UTR) | | | | | | | |
| | | | AGGCGGCGTGGAGATC | (SEQ ID NO:72) | | | | | |
| | | | VIC-CTCCCGGCACGCC-NFQ | (SEQ ID NO:73) | | | | | |
| | | | FAM-CTCCCCGCACGCC-NFQ | (SEQ ID NO:74) | | | | | |
| 68 | rs11809982 | ZSWIM5 | C_{——}1506165_10 | | 43771496 | 44901506 | 27.0 | 0.003 | 0.083 |
| 69 | rs2036426 | ZSWIM5 | C_12105318_10 | | 43794389 | 44924393 | 7.7 | 1.000 | 0.365 |
| 70 | rs1226749 | ZSWIM5 | TCACAGTTTAGAGCAGTTAAACAAAGGA | (SEQ ID NO:75) | 43921776 | 45051780 | 14.4 | 0.177 | 0.008 |
| | | | AGGCACAACATTCTGAAGAGTGATT | (SEQ ID NO:76) | | | | | |
| | | | VIC-AAGAATGATTTGCATAATAA-NFQ | (SEQ ID NO:77) | | | | | |
| | | | FAM-AGAATGATTTGCGTAATAA-NFQ | (SEQ ID NO:78) | | | | | |
| 71 | rs11576668 | BC006119 | C_{——}9168020_10 | | 44053549 | 45183461 | 10.6 | 0.481 | 0.512 |
| 72 | rs7544178 | TESK.2 | C_{——}479587_10 | | 44102443 | 45232363 | 24.7 | 1.000 | 0.284 |
| 73 | rs1417578 | TESK2 | C_{——}331583_10 | | 44133891 | 45263884 | 25.5 | 1.000 | 0.181 |
| 74 | rs781062 | TESK2 | C_12109356_10 | | 44216045 | 45346032 | 27.1 | 0.477 | 0.660 |
| 75 | rs781061 | TESK2 | TGATGGACTGCCAATAATATTTTTGTTTCC | (SEQ ID NO:79) | 44216194 | 45346181 | 26.6 | 0.278 | 0.544 |
| | | | GCAGAAAAGAGTACAGTATAATAAATAACACCCA | (SEQ ID NO:80) | | | | | |
| | | | VIC-CATTTTGTGTTATTTGCC-NFQ | (SEQ ID NO:81) | | | | | |
| | | | FAM-ATTTTGTGTTGTTTGCC-NFQ | (SEQ ID NO:82) | | | | | |
| 76 | rs12743512 | TESK2 | C_{——}1238861_10 | | 44237353 | 45367327 | 43.3 | 0.239 | 0.525 |
| 77 | rs3014216 | | C_11869471_10 | | 44319745 | 45449054 | 44.3 | 0.880 | 0.798 |
| 78 | rs6656279 | SP192 | C_{——}482652_10 | | 44408070 | 45537382 | 44.3 | 1.000 | 0.714 |
| 79 | rs6658700 | | C_434443_10 | | 44444241 | 45573540 | 28.7 | 1.000 | 0.080 |
| 80 | rs10437063 | MAST2 | C_{———}518427_10 | | 44561309 | 45643583 | 28.7 | 0.737 | 0.340 |
| 81 | rs6686134 | MAST2 | C_{———}167598_10 | | 44665571 | 45748185 | 42.2 | 0.466 | 1.000 |
| 82 | rs1707336 | MAST2 | C_{——}8358540_1_{—} | | 44780753 | 45863377 | 42.2 | 0.555 | 0.899 |
| 83 | rs785467 | PIK3R3 | C_{——}1595972_1_{—} | | 44808850 | 45891476 | 27.9 | 1.000 | 0.202 |
| 84 | rs1473840 | | C_{——}1595904_10 | | 44888498 | 45971114 | 32.3 | 0.870 | 0.519 |
| 85 | rs12028248 | AK057892 | C_{——}1595867_10 | | 44978075 | 46060248 | 23.5 | 0.846 | 1.000 |
| 86 | rs10890388 | MUF1 | C_{——}3159725_10 | | 45048876 | 46131413 | 24.4 | 1.000 | 0.198 |
| 87 | rs11588062 | UQCRH | CCAATTTTCCATCCATAGATGCAAAGATT | (SEQ ID NO:83) | | 46149681 | 29.8 | 0.611 | 0.767 |
| | | | CTTGGCCTCCCAAAGTGTTG | (SEQ ID NO:84) | | | | | |
| | | | VIC-CCCCGGCCCCCTT | (SEQ ID NO:85) | | | | | |
| | | | FAM-CCCCAGCCCCCTT | (SEQ ID NO:86) | | | | | |
| 88 | rs4660920 | UQCRH | TGGATAAACCTTGCAAACATGC | (SEQ ID NO:87) | 45068842 | 46151379 | 24.8 | 0.188 | 0.454 |
| | | | GGGAACAGATCATGACTTGCCTA | (SEQ ID NO:88) | | | | | |
| | | | FAM-ATATGATTTGTATGAAATGT-NFQ | (SEQ ID NO:89) | | | | | |
| | | | VIC-TATGATTTCTATGAAATGTTNFQ | (SEQ ID NO:90) | | | | | |
| 89 | rs4660921 | UQCRH | TTTGTCAGCCAAGCACTGGTT | (SEQ ID NO:91) | 45068982 | 46151519 | 27.4 | 0.858 | 1.000 |
| | | | GCTCATAAACTCAGTGAAGGAATGAA | (SEQ ID NO:92) | | | | | |
| | | | FAM-ATCTGGgAGTAAGATAG-NFQ | (SEQ ID NO:93) | | | | | |
| | | | VIC-ATCTGGtAGTAAGATAGAC-NFQ | (SEQ ID NO:94) | | | | | |
| 90 | rs324420 | FAAH | C_{——}1897306_10 | | 45158121 | 46240678 | 19.9 | 0.403 | 0.848 |
| 91 | rs12132747 | OTX3 | C_{——}1897131_10 | | 45262684 | 46345211 | 21.1 | 0.818 | 0.557 |
| 92 | rs1933934 | MKNK1 | C_{——}11729224_10 | | 45322305 | 46404845 | 27.7 | 0.110 | 0.463 |
| 93 | rs614486 | BC057818 | C_{———}809542_10 | | 45426170 | 46508736 | 27.6 | 0.057 | 0.882 |
| 94 | rs2297810 | CYP4B1 | C_16187548_10 | | 45568234 | 46650776 | 11.6 | 1.000 | 0.054 |
| 95 | rs2297809 | CYP4B1 | C_{——}16187547_10 | | 45570147 | 46652689 | 11.5 | 1.000 | 0.115 |
| 96 | rs6429627 | | CTGCCTGCTATCTGTCATCTTCA | (SEQ ID NO:95) | 45671404 | 46753946 | 22.5 | 1.000 | 0.164 |
| | | | GTCCTGGCCAAAGCAATCAG | (SEQ ID NO:96) | | | | | |
| | | | VIC-CAAGAGGAAGACATAGTT-NFQ | (SEQ ID NO:97) | | | | | |
| | | | FAM-AGAGGAAGGCATAGTT-NFQ | (SEQ ID NO:98) | | | | | |
| 97 | rs6669062 | | C_{———}163689_10 | | 45755653 | 46838386 | 25.5 | 1.000 | 0.260 |
| 98 | rs6675902 | CYP4Z1 | C_11871078_10 | | 45859347 | 46941421 | 33.0 | 0.740 | 0.291 |
| 99 | rs941412 | | C_{——}2808085_10 | | 45944961 | 47028609 | 21.8 | 0.848 | 0.213 |
| 100 | rs11577960 | SIL | C_11871209_{——}10 | | 46035124 | 47118769 | 31.6 | 0.860 | 1.000 |
| 101 | rs6795 | UMP-CMPK | C_12102717_10 | | 46130734 | 47214381 | 47.4 | 0.320 | 0.019 |
| 102 | rs564914 | | C_{———}552994_10 | | 46201531 | 47285150 | 45.1 | 0.063 | 0.048 |
| 103 | rs513464 | | GGCCCCTCTCCGTGGAT | (SEQ ID NO:99) | 46267361 | 47350913 | 10.0 | 0.430 | 0.102 |
| | | | TTAGGCATTTGCTTCTTTATCTGA | (SEQ ID NO:100) | | | | | |
| | | | FAM-TCTCCCTCCTGCTCTCATACCACCC-BHQ1 | (SEQ ID NO:101) | | | | | |
| | | | TET-TCTCCCTCCTGCTTTCATACCACCC-BHQ1 | (SEQ ID NO:102) | | | | | |
| 104 | rs893762 | | GTGGCAGAAGTAGCACTGAGA | (SEQ ID NO:103) | 46406354 | 47489906 | 7.4 | 1.000 | 0.644 |
| | | | GCCACAGAGGGAACTTGTTTTTAAC | (SEQ ID NO:104) | | | | | |
| | | | VIC-CAGAGAAAGTGACAGATT-NFQ | (SEQ ID NO:105) | | | | | |
| | | | FAM-AACAGAGAAAGTAACAGATT-NFQ | (SEQ ID NO:106) | | | | | |
| 105 | rs1079181 | | C_{——}1053545_10 | | 46464292 | 47547844 | 2.1 | 1.000 | 0.279 |
| 106 | rs2282361 | | C_{——}1053541_1_{—} | | 46526807 | 47609922 | 49.2 | 0.573 | 1.000 |
| 107 | rs1538779 | | C_11285422_10 | | 46600632 | 47683753 | 32.5 | 0.250 | 0.889 |
| 108 | rs303913 | | C_{———}701909_10 | | 46737114 | 47820279 | 8.3 | 1.000 | 0.206 |
| 109 | rs823385 | | C_{——}7554154_1_{—} | | 46801354 | 47884416 | 46.9 | 0.029 | 0.712 |
| 110 | rs10788882 | | C_{——}3027932_10 | | 46917248 | 48000355 | 29.0 | 0.130 | 0.399 |
| 111 | rs550663 | | C_{——}2809699_10 | | 47011013 | 48094154 | 27.7 | 0.109 | 1.000 |
| 112 | rs6700461 | spata6 | C_{——}1575325_10 | | 47081817 | 48165024 | 43.5 | 0.370 | 0.711 |
| 113 | rs3738309 | spata6 | C_{———}473660_1_{—} | | 47155632 | 48239205 | 43.6 | 0.083 | 0.133 |
| 114 | rs2485911 | spata6 | C_11873394_10 | | 47197325 | 48280893 | 28.1 | 0.158 | 1.000 |
| 115 | rs2798125 | | C_{———}193129_10 | | 47326438 | 48410301 | 35.6 | 0.214 | 0.474 |
| 116 | rs320029 | FLJ14442 | C_{——}3146199_10 | | 47371754 | 48455620 | 40.9 | 0.227 | 0.462 |
| 117 | rs561383 | FLJ14442 | C_{———}959821_10 | | 47424205 | 48508096 | 44.6 | 1.000 | 0.383 |
| 118 | rs10888617 | FLJ14442 | C_{——}1962672_10 | | 47470996 | 48554905 | 45.9 | 0.552 | 0.901 |
| 119 | rs6664435 | FLJ14442 | C_{———}203871_10 | | 47524743 | 48608667 | 31.6 | 0.863 | 0.755 |
| 120 | rs1934404 | FLJ14442 | C_11727910_10 | | 47583457 | 48667410 | 20.9 | 0.271 | 0.558 |
| 121 | rs11205566 | FLJ14442 | C_{———}393112_10 | | 47633357 | 48717307 | 38.1 | 0.766 | 0.789 |
| 122 | rs959145 | FLJ14442 | C_{——}8853273_10 | | 47687088 | 48771031 | 10.3 | 1.000 | 0.761 |
| 123 | rs1925425 | FLJ14442 | C_{——}1964081_10 | | 47731309 | 48815251 | 41.9 | 1.000 | 0.447 |
| 124 | rs1361544 | FLJ14442 | C_{——}8853256_10 | | 47777318 | 48861294 | 11.3 | 0.732 | 1.000 |
| 125 | rs3905053 | | C_{———}434038_10 | | 47818641 | 48902617 | 37.1 | 0.758 | 0.685 |
| 126 | rs355206 | | C_{——}3205907_10 | | 47958113 | 49042091 | 32.1 | 0.620 | 0.398 |
| 127 | rs1431638 | | C_{——}3205878_10 | | 48048326 | 49132335 | 36.2 | 0.879 | 0.909 |
| 128 | rs1167272 | | CCAATACAGAGCACTTTTACATTCATTA | (SEQ ID NO:107) | 48171895 | 49255904 | 31.6 | 0.868 | 0.582 |
| | | | AGGTATGAAATTGGGTGTATTGCTAA | (SEQ ID NO:108) | | | | | |
| | | | FAM-TGGAGTGAGGCAAACTAAGTCCCAGAA-BHQ1 | (SEQ ID NO:109) | | | | | |
| | | | TET-AGTGAGGCAAACTGAGTCCCAGAAACTC-BHQ1 | (SEQ ID NO:110) | | | | | |
| 129 | rs1415985 | | CACAAAGAACACTGGCATTTTAAGA | (SEQ ID NO:111) | 48216657 | 49300666 | 43.0 | 1.000 | 0.794 |
| | | | TTCTCAAAATAGCTCCACAGTGTATGT | (SEQ ID NO:112_{—} | | | | | |
| | | | FAM-ACCAAACAAAGCAGAATGTCAGGCC-BHQ1 | (SEQ ID NO:113) | | | | | |
| | | | TET-CCAAACAAAGTAGAATGTCAGGCCCTG-BHQ1 | (SEQ ID NO:114) | | | | | |
| 130 | rs2103266 | | CGGAGCTGCCTGCTAGTC | (SEQ ID NO:115) | 48308281 | 49392290 | 35.9 | 0.753 | 0.701 |
| | | | GCCCAAGGGCTGAAGAGT | (SEQ ID NO:116) | | | | | |
| | | | VIC-CAGTGCTAGGTGCCG-NFQ | (SEQ ID NO:117) | | | | | |
| | | | FAM-CAGTGCTAAGTGCCG-NFQ | (SEQ ID NO:118) | | | | | |
| 131 | rs1343161 | | C_{———}118289_10 | | 48396710 | 49480767 | 31.4 | 0.608 | 0.391 |
| 132 | rs7364999 | | CCCTGTTTGCCTGGATGTCA | (SEQ ID NO:119) | 48506057 | 49590114 | 31.5 | 0.753 | 0.478 |
| | | | GGAGCAGGCAGCAATCTTTG | (SEQ ID NO:120) | | | | | |
| | | | VIC-CTGTTGCACAGGCT-NFQ | (SEQ ID NO:121) | | | | | |
| | | | FAM-CTGTTGCGCAGGCT-NFQ | (SEQ ID NO:122) | | | | | |
| 133 | rs6693846 | | ACCACTCTACTGCAAGTCTCATGTA | (SEQ ID NO:123) | 48601212 | 49685269 | 31.0 | 0.513 | 0.486 |
| | | | TCACCAAATAAATAATGCATATTTTCCCAACAAT | (SEQ ID NO:124) | | | | | |
| | | | VIC-CTGATACAACCAATTATTCATA-NFQ | (SEQ ID NO:125) | | | | | |
| | | | FAM-TGATACAACCAATTGTTCATA-NFQ | (SEQ ID NO:126) | | | | | |
| 134 | rs12725018 | | C_{——}500007_10 | | 48741182 | 49825243 | 31.9 | 0.323 | 0.200 |
| 135 | rs7520915 | | C_{———}109654_10 | | 48841577 | 49925579 | 39.6 | 0.654 | 0.293 |
| 136 | rs967582 | | C_{——}1406377_{——}10 | | 48868089 | 49952089 | 36.4 | 0.826 | 0.074 |
| 137 | rs5000809 | ELAVL4 | C_{———}92611_10 | | 48882375 | 49966374 | 31.9 | 0.238 | 0.234 |
| 138 | rs3902720 | ELAVL4 | C_{——}1406360_10 | | 48891263 | 49975254 | 31.6 | 0.554 | 0.054 |
| 139 | rs4412638 | ELAVL4 | C_{———}432130_10 | | 48899602 | 49983593 | 27.4 | 0.093 | 0.542 |
| 140 | rs10888681 | ELAVL4 | C_{——}1406368_10 | | 48903216 | 49987207 | 31.8 | 0.168 | 0.128 |
| 141 | rs1018670 | ELAVL4 | C_{——}1406371_10 | | 48923480 | 50007471 | 32.6 | 0.169 | 0.110 |
| 142 | rs3009113 | ELAVL4 | C_{——}1406373_10 | | 48935628 | 50019629 | 41.1 | 0.348 | 0.480 |
| 143 | rs2494876 | ELAVL4 | GTGTGTTATCCTTGGTCAGACTGATG | (SEQ ID NO:127) | 48952089 | 50036432 | 10.5 | 1.000 | 0.244 |
| | | | CTGTGTGACCAGGGATGTTCATT | (SEQ ID NO:128) | | | | | |
| | | | TET-CCTTCTGCTTGTCCCCCCAGGTTCT-BHQ1 | (SEQ ID NO:129) | | | | | |
| | | | FAM-CCTTCTGCTTGTTCCCCCAGGTTC-BHQ1 | (SEQ ID NO:130 | | | | | |
| 144 | rs1948808 | | C_12108074_10 | | 49080212 | 50164213 | 45.6 | 0.781 | 0.902 |
| 145 | rs1278527 | | C_{——}7618775_10 | | 49176861 | 50260885 | 42.3 | 1.000 | 0.318 |
| 146 | rs3862271 | FAF1 | C_{———}576976_10 | | 49240891 | 50324418 | 26.7 | 0.790 | 0.326 |
| 147 | rs12568008 | FAF1 | C_11302783_10 | | 49362716 | 50446740 | 7.5 | 1.000 | 0.641 |
| 148 | rs11587750 | FAF1 | C_11860065_10 | | 49436570 | 50520097 | 24.2 | 0.583 | 0.919 |
| 149 | rs1416685 | FAF1 | C_{———}216050_10 | | 49529765 | 50613292 | 37.3 | 1.000 | 0.898 |
| 150 | rs1398868 | FAF1 | C_{——}9509099_10 | | 49605735 | 50689264 | 27.9 | 0.813 | 0.918 |
| 151 | rs12855 | CDKN2C | C_{——}8847082_10 | | 49726604 | 50810011 | 10.0 | 0.438 | 0.708 |
| 152 | rs6588399 | | CACACACACACACACACACATTAT | (SEQ ID NO:131) | 49876046 | 50959573 | 21.1 | 1.000 | 0.836 |
| | | | GGCTGGGAAAAAATATTTGCAAAGTACATA | (SEQ ID NO:132) | | | | | |
| | | | VIC-TCGCTCTCTCTCTCTATATA-NFQ | (SEQ ID NO:133) | | | | | |
| | | | FAM-CGCTCTCTCTCTATATATA-NFQ | (SEQ ID NO:134) | | | | | |
| 153 | rs7526029 | RNF11 | TCTCTGCTGATTTGTCATGTACAGTTT | (SEQ ID NO:135) | 49995312 | 51078701 | 9.5 | 0.375 | 1.000 |
| | | | GATGTGGAGAAACAACTGTTAAAGCA | (SEQ ID NO:136) | | | | | |
| | | | VIC-ATCTGGAAATCATATATTG-NFQ | (SEQ ID NO:137) | | | | | |
| | | | FAM-TCTGGAAATCGTATATTG-NFQ | (SEQ ID NO:138) | | | | | |
| 154 | rs6701572 | RNF11 | C_{——}1413758_10 | | 50005845 | 51089233 | 9.1 | 0.324 | 0.802 |
| 155 | rs616055 | RNF11 | C_{———}937775_10 | | 50020915 | 51104304 | 15.9 | 1.000 | 0.773 |
| 156 | rs17567 | EPS15 | C_{——}11740230_10 | | 50113450 | 51196839 | 26.9 | 0.368 | 0.139 |
| 157 | rs6694583 | EPS15 | C_{——}3125026_10 | | 50250286 | 51333681 | 26.8 | 0.353 | 0.144 |
| 158 | rs1316981 | | C_{———}386562_10 | | 50321582 | 51404976 | 28.8 | 0.357 | 0.902 |
| 159 | rs7524425 | OSBPL9 | C_{———}519863_10 | | 50438644 | 51522025 | 14.8 | 0.772 | 1.000 |
| 160 | rs1770791 | NRD1 | C_{——}8847889_1_{—} | | 50550601 | 51633982 | 24.5 | 0.548 | 0.635 |
| 161 | rs10888734 | NRD1 | C_{——}2776353_1_{—} | | 50552779 | 51636160 | 46.9 | 0.775 | 0.138 |
| 162 | rs11205896 | NRD1 | C_{——}2776339_10 | | 50577600 | 51660902 | 47.1 | 0.668 | 0.177 |
| 163 | rs3765687 | RAB3B | C_11865895_10 | | 50689440 | 51772024 | 47.7 | 0.473 | 0.193 |
| 164 | rs7529324 | TLP19 | C_{——}1805290_10 | | 50804888 | 51887330 | 13.7 | 0.094 | 0.117 |
| 165 | rs10888748 | MADHIP | C_{——}1918486_10 | | 50915767 | 51998207 | 13.2 | 0.522 | 0.220 |
| 166 | rs3790522 | MADHIP | C_{———}251124_10 | | 50991996 | 52075345 | 8.5 | 1.000 | 0.336 |
| 167 | rs2762818 | MADHIP | C_{——}1914956_10 | | 51085710 | 52168931 | 8.3 | 1.000 | 0.306 |
| 168 | rs9633423 | | C_{——}1914945_10 | | 51122833 | 52206057 | 28.6 | 0.397 | 0.693 |
| 169 | rs2274147 | D83776 | C_{——}1918085_1_{—} | | 51187521 | 52270741 | 26.0 | 0.707 | 0.740 |
| 170 | rs835036 | BC048301 | CATCTTCTGGGCATACCACAGT | (SEQ ID NO:139) | 51283938 | 52367158 | 28.5 | 0.076 | 0.405 |
| | | | TCTTTTGGATTTCATGTATTTTTAAAGTGTGAACA | (SEQ ID NO:140) | | | | | |
| | | | VIC-TTTATTGGGTGCCTACTTT-NFQ | (SEQ ID NO:141) | | | | | |
| | | | FAM-TGGGTGCCTGCTTT-NFQ | (SEQ ID NO:142) | | | | | |
| 171 | rs1970951 | GPX7 | C_11730536_1_{—} | | 51359148 | 52442372 | 19.3 | 0.673 | 0.283 |
| 172 | rs6588434 | MGC52498 | C_11875165_10 | | 51397518 | 52480679 | 33.0 | 0.410 | 0.435 |
| 173 | rs443751 | FLJ12439 | C_{——}1755656_10 | | 51440196 | 52523350 | 39.2 | 0.068 | 0.488 |
| 174 | rs6588441 | AB0515617 | C_{——}1755700_10 | | 51510244 | 52593412 | 42.7 | 0.881 | 0.902 |
| 175 | rs554301 | | C_{——}1643943_10 | | 51609408 | 52691866 | 41.7 | 0.655 | 0.536 |
| 176 | rs7548389 | SCP2 | C_{———}170668_10 | | 51692186 | 52774129 | 37.8 | 1.000 | 1.000 |
| 177 | rs12747412 | SCP2 | C_{——}7838616_10 | | 51791259 | 52873200 | 40.7 | 0.871 | 0.691 |
| 178 | rs899974 | PODN | C_{——}8329979_1_{—} | | 51838159 | 52920105 | 3.9 | 1.000 | 1.000 |
| 179 | rs899976 | SLC1A7 | C_{——}7842292_10 | | 51881768 | 52963731 | 25.8 | 0.713 | 0.271 |
| 180 | rs1799821 | CPT2 | C_{——}1797305_1_{—} | | 51964290 | 53046366 | 46.4 | 0.553 | 0.084 |
| 181 | rs5174 | LRP8 | C_{———}190754_10 | | 52000573 | 53082645 | 42.8 | 0.317 | 0.121 |
| 182 | rs2782497 | | C_15933601_10 | | 52096339 | 53178948 | 30.4 | 0.182 | 0.586 |
| 183 | rs1288599 | AK097753 | C_12108624_10 | | 52192317 | 53274900 | 15.2 | 0.002 | 0.829 |
| 184 | rs496933 | FLJ36155 | C_{——}3176687_10 | | 52296963 | 53379197 | 28.6 | 0.393 | 0.398 |
| 185 | rs7551844 | FLJ36155 | C_{——}7836297_{——}10 | | 52349017 | 53431251 | 30.1 | 0.238 | 1.000 |
| 186 | rs3013777 | FLJ36155 | TGTCCATCACCTAACTGAACTTCCT | (SEQ ID NO:143) | 52440305 | 53522539 | 38.7 | 1.000 | 0.160 |
| | | | CACTGTGTACCAGGGCAAAGA | (SEQ ID NO:144) | | | | | |
| | | | VIC-AGGGCTCaACACTG-NFQ | (SEQ ID NO:145) | | | | | |
| | | | FAM-AAGGGCTCgACACTG-NFQ | (SEQ ID NO:146) | | | | | |
| 187 | rs1569783 | FLJ10407 | C_{——}8328074_10 | | 52534976 | 53617189 | 15.7 | 0.431 | 0.451 |
| 188 | rs3817871 | DJ167A19.1 | C_{——}2494217_10 | | 52642605 | 53724853 | 16.1 | 0.438 | 0.443 |
| 189 | rs1063162 | MGC8974 | C_{——}7547909_10 | | 52699869 | 53782099 | 17.3 | 0.441 | 0.683 |
| 190 | rs914720 | | C_{——}7547859_10 | | 52772159 | 53854400 | 45.4 | 0.662 | 0.433 |
| 191 | rs7528837 | C1orf8 | GCTTTTCCAGTATGAGAGTAGCTTTAAGA | (SEQ ID NO:147) | 52787873 | 53870103 | 1.8 | 0.043 | 0.254 |
| | | | CGAACTCCTGACCTCAAGTGATTC | (SEQ ID NO:148) | | | | | |
| | | | VIC-AGTGGCTCACACCTGT-NFQ | (SEQ ID NO:149) | | | | | |
| | | | FAM-TGGCTCACGCCTGT-NFQ | (SEQ ID NO:150) | | | | | |
| 192 | rs3766466 | C1orf8 | AGCAGAAACTTGTTTACCACTCACT | (SEQ ID NO:151) | 53875355 | 2.0 | 0.037 | 0.227 | |
| | | | AGAGAAAGATAGTGGGCCATACCA | (SEQ ID NO:152) | | | | | |
| | | | VIC-TCACCTACTCGGTGTCAG-NFQ | (SEQ ID NO:153) | | | | | |
| | | | FAM-TATCACCTACTCTGTGTCAG-NFQ | (SEQ ID NO:154) | | | | | |
| 193 | rs914722 | C1orf8 | CACATGGCAAATGGTGACACAA | (SEQ ID NO:155) | 52801515 | 53883745 | 35.6 | 1.000 | 0.208 |
| | | | GTAAGCCCAGTTTTAAAAAATCCCTTCA | (SEQ ID NO:156) | | | | | |
| | | | VIC-CCTTACTTTATCAGGCCC-NFQ | (SEQ ID NO:157) | | | | | |
| | | | FAM-CTTACTTTTTCAGGCCC-NFQ | (SEQ ID NO:158) | | | | | |
| 194 | rs2236512 | C1orf8 | CAACCATCGCAAGCGTTAGC | (SEQ ID NO:159) | | 53889025 | 2.3 | 0.004 | 1.000 |
| | | | CCCCGCGAAGGGAAGAAG | (SEQ ID NO:160) | | | | | |
| | | | VIC-TCAGGAGGCCCCGCT-NFQ | (SEQ ID NO:161) | | | | | |
| | | | FAM-AGGAGGCGCCGCT-NFQ | (SEQ ID NO:162) | | | | | |
| 195 | hcv1452882 | LOC200008 | C_{——}1452882_10 | | 52897356 | 53979607 | 35.9 | 0.344 | 0.603 |
| 196 | rs13571 | MRPL37 | C_{——}2206322_1_{—} | | 52969546 | 54051838 | 23.7 | 0.541 | 1.000 |
| 197 | rs646534 | SSBP3 | C_{——}2431627_10 | | 53022287 | 54104656 | 46.4 | 0.559 | 0.795 |
| 198 | rs3927580 | SSBP3 | C_{——}11870668_10 | | 53072252 | 54154634 | 22.4 | 0.048 | 0.201 |
| 199 | rs4927095 | SSBP3 | C_{——}2801176_10 | | 53110290 | 54192533 | 15.2 | 0.056 | 0.586 |
| 200 | rs213501 | SSBP3 | C_{——}3025515_10 | | 53150346 | 54232588 | 37.1 | 1.000 | 0.298 |
| 201 | rs910112 | | CCAAGGACCTCCATAAATAGTGACA | (SEQ ID NO:163) | 53213457 | 54295699 | 5.6 | 0.399 | 0.604 |
| | | | ACAGAGGTAGGGCTGCAACTG | (SEQ ID NO:164) | | | | | |
| | | | FAM-CATGACTTTGCAAGAGACCAGAAGCATT-BHQ1 | (SEQ ID NO:165) | | | | | |
| | | | TET-ATGACTTTGCAAGAGGCCAGAAGCAT-BHQ1 | (SEQ ID NO:166) | | | | | |
| | IMS- | | | | | | | | |
| 202 | JST105898 | THEA | C_{——}3025495_10 | | 53301715 | 54384057 | 28.4 | 0.141 | 0.453 |
| 203 | rs1702003 | THEA | C_{——}7549360_1_{—} | 53347938 | 54430280 | 3.1 | 1.000 | 1.000 | |
| 204 | rs644955 | FLJ46354 | C_{———}970030_10 | | 53455678 | 54538002 | 48.5 | 1.000 | 0.802 |
| 205 | rs1147990 | TTC4 | C_{——}3154981_10 | | 53469894 | 54552218 | 49.0 | 0.381 | 0.174 |
| 206 | rs3766415 | TTC4 | GTCTTGGCCTGTTCTGCAAAG | (SEQ ID NO:167) | 53470726 | 54553050 | 6.8 | 0.603 | 1.000 |
| | | | GGTGTGTCATATAGTACATTATTACATGATTTAGAAT | (SEQ ID NO:168) | | | | | |
| | | | CTATTTT | | | | | | |
| | | | VIC-ATAATCACTATTGCTTACTTTT-NFQ | (SEQ ID NO:169) | | | | | |
| | | | FAM-CACTATTGCCTACTTTT-NFQ | (SEQ ID NO:170) | | | | | |
| 207 | rs3737825 | TTC4 | C_{——}3154985_1_{—} | | 53474519 | 54556843 | 6.7 | 0.602 | 1.000 |
| 208 | rs4926653 | TTC4 | C_{——}3155005_10 | | 53483691 | 54566017 | 49.0 | 0.080 | 0.214 |
| 209 | rs11206424 | TTC4 | GGAGCAAGTCACCTCTTACGT | (SEQ ID NO:171) | | 54573462 | 6.5 | 1.000 | 1.000 |
| | | | TTCCTGCACAAGCTCTCTCTTTT | (SEQ ID NO:172) | | | | | |
| | | | VIC-ATGGCGGAAGGCA | (SEQ ID NO:173) | | | | | |
| | | | FAM-ATGGCAGAAGGCA | (SEQ ID NO:174) | | | | | |
| | | DKFZP727A | | | | | | | |
| 210 | rs2270004 | 071 | C_{——}3155029_1 | | 53511728 | 54594049 | 15.0 | 0.083 | 1.000 |
| 211 | rs4926658 | FLJ40201 | C_{——}2636133_10 | | 53570776 | 54652994 | 33.7 | 1.000 | 0.151 |
| 212 | rs7374 | DHCR24 | C_{——}2794200_1_{—} | | 53603987 | 54686240 | 31.3 | 0.869 | 0.332 |
| 213 | rs638944 | DHCR24 | C_{——}2794232_10 | | 53629520 | 54711833 | 43.7 | 0.550 | 0.211 |
| 214 | rs2433675 | LOC199964 | C_{——}2794414_10 | | 53735658 | 54817971 | 21.1 | 0.192 | 0.229 |
| 215 | hcv201363 | BSND | C_{———}201363_10 | | 53761870 | 54844180 | 20.7 | 0.193 | 0.474 |
| 216 | rs1165287 | PCSK9 | C_{——}3184726_10 | | 53807832 | 54890130 | 33.8 | 0.441 | 0.901 |
| 217 | rs516499 | PCSK9 | C_{——}3184712_10 | | 53814289 | 54896603 | 13.8 | 1.000 | 0.620 |
| | | USP24 | AGCAACATGATCTGAAGCGTATAATATAC | | | | | | |
| 218 | rs13312 | (3′UTR) | | (SEQ ID NO:175) | 53820346 | 54902660 | 18.1 | 0.480 | 0.525 |
| | | | GCCACTTCTAGTCCCCTTATTTCC | (SEQ ID NO:176) | | | | | |
| | | | FAM-CGATCCTGATGAAGCTTTACAGTGAGGA-BHQ1 | (SEQ ID NO:177) | | | | | |
| | | | TET-CGATCCTGATGAACCTTTACAGTGAGGA-BHQ1 | (SEQ ID NO:178) | | | | | |
| 219 | rs1043671 | USP24 | CAATACCAAGGGTTTTCAGTAATTATGTT | (SEQ ID NO:179) | 53821415 | 54903729 | 4.1 | 1.000 | 1.000 |
| | | (3′UTR) | | | | | | | |
| | | | GCTTGGAGACATATTGAATAAACTGTAGTC | (SEQ ID NO:180) | | | | | |
| | | | FAM-AGCAAACGATTGCAGATCACATGATTTAA-BHQ1 | (SEQ ID NO:181) | | | | | |
| | | | TET-AGCAAACGATTGCAGACCACATGATT-BHQ1 | (SEQ ID NO:182) | | | | | |
| | | USP24 | | | | | | | |
| 220 | rs487230 | (A286V) | C_{——}3184710_1_{—} | | 53828772 | 54911092 | 22.7 | 0.683 | 0.114 |
| 221 | rs683880 | USP24 | C_{———}998732_1_{—} | | 53834484 | 54916813 | 22.1 | 1.000 | 0.385 |
| 222 | rs667353 | USP24 | C_11289191_1_{—} | | 53845130 | 54927458 | 36.8 | 0.880 | 1.000 |
| 223 | rs615652 | USP24 | C_{——}3184701_10 | | 53854998 | 54937328 | 12.8 | 0.755 | 0.804 |
| 224 | rs594226 | AK127075 | C_{———}998715_1_{—} | | 53860456 | 54942785 | 22.5 | 0.698 | 0.081 |
| 225 | rs567734 | AK127075 | C_{———}998713_10 | | 53861957 | 54944282 | 18.8 | 0.830 | 0.335 |
| 226 | rs625219 | AK127075 | C_11732132_10 | | 53873282 | 54955599 | 13.3 | 0.760 | 1.000 |
| 227 | rs1165226 | AK127075 | C_11732134_10 | | 53895603 | 54977923 | 38.1 | 0.457 | 0.708 |
| 228 | rs1024305 | | C_{——}7548615_10 | | 53917799 | 55000122 | 18.8 | 0.817 | 0.323 |
| 229 | rs287234 | | CTCCTTACTAACGTAGAGCTCACCTA | (SEQ ID NO:183) | 53954100 | 55036438 | 4.6 | 1.000 | 1.000 |
| | | | ACACAAGAAAGAACATAGTGGATGCT | (SEQ ID NO:184) | | | | | |
| | | | VIC-AAACCCTTTTTAAGCCTTTA-NFQ | (SEQ ID NO:185) | | | | | |
| | | | FAM-AAACCCTTTTTAAACCTTTA-NFQ | (SEQ ID NO:186) | | | | | |
| 230 | rs287235 | | C_{———}686425_10 | | 53966079 | 55048417 | 23.0 | 1.000 | 0.735 |
| 231 | rs2047422 | | CGTGCCTGTTTGTTGCTTAAATG | (SEQ ID NO:187) | 53999547 | 55081885 | 40.2 | 0.873 | 0.132 |
| | | | AGACCAAGGGATAAACAGTTGAAAAGT | (SEQ ID NO:188) | | | | | |
| | | | VIC-TATTCTCACATATTTATCATTGTT-NFQ | (SEQ ID NO:189) | | | | | |
| | | | FAM-TCACATATTTGTCATTGTT-NFQ | (SEQ ID NO:190) | | | | | |
| 232 | rs2047418 | | CCCACCTGGAGATTCTGACTCA | (SEQ ID NO:191) | 54030679 | 55113017 | 21.4 | 1.000 | 0.269 |
| | | | CTCCCTCCCTTCATCAGTTGTTC | (SEQ ID NO:192) | | | | | |
| | | | VIC-CCACCCAGACCCAG-NFQ | (SEQ ID NO:193) | | | | | |
| | | | FAM-CCACCCACACCCAG-NFQ | (SEQ ID NO:194) | | | | | |
| 233 | rs10493202 | | AGAATTCAATATGGTGAGATGAATGC | (SEQ ID NO:195) | 54051686 | 55134024 | 15.0 | 0.773 | 1.000 |
| | | | ATCCTCTGAACTGTTCTGAGTGTCA | (SEQ ID NO:196) | | | | | |
| | | | FAM-TGCCAAACCCAAGCTGAAAGGC-BHQ1 | (SEQ ID NO:197 | | | | | |
| | | | TET-TGCCAAACCCACGCTGAAAGG-BHQ1 | (SEQ ID NO:198) | | | | | |
| 234 | rs207150 | | GTGCTCTGATAGCACCAGTGAGA | (SEQ ID NO:199) | 54094045 | 55176383 | 6.5 | 0.123 | 0.393 |
| | | | GACTGGCAACTTCTTTTAACATTACCT | (SEQ ID NO:200) | | | | | |
| | | | FAM-AGGCCTAAACCCTAGAATTGGCAATGA-BHQ1 | (SEQ ID NO:201) | | | | | |
| | | | TET-AGGCCTAAACCCTGGAATTGGCA-BHQ1 | (SEQ ID NO:202) | | | | | |
| 235 | rs12565257 | | C_{——}2524674_10 | | 54124661 | 55205348 | 37.9 | 0.884 | 0.180 |
| 236 | rs2015252 | | C_{——}2524652C_10 | | 54161982 | 55242698 | 44.5 | 0.760 | 0.459 |
| 237 | rs904610 | | TGCCCATTACATGCCTGACA | (SEQ ID NO:203) | 54276994 | 55359332 | 24.4 | 0.308 | 0.493 |
| | | | CCAGGTAAACAAACAAATATGATATCG | (SEQ ID NO:204) | | | | | |
| | | | FAM-TGTCTCAAGAGTTGAGTGGGGAAGACA-BHQ1 | (SEQ ID NO:205) | | | | | |
| | | | TET-CTGTCTCAAGAGTTGATTGGGGAAGACA-BHQ1 | (SEQ ID NO:206) | | | | | |
| 238 | rs1514135 | AK127270 | GCCAGAAATCCTACTCTTTGGGAAA | (SEQ ID NO:207) | 54403812 | 55486150 | 37.1 | 0.436 | 0.187 |
| | | | AGCAGAAGTTTGGATGGAGGAAAA | (SEQ ID NO:208) | | | | | |
| | | | VIC-CAAATGCTGCAAGTAC-NFQ | (SEQ ID NO:209) | | | | | |
| | | | FAM-CAAATGCTGGAAGTAC-NFQ | (SEQ ID NO:210) | | | | | |
| 239 | rs753978 | | CTGGGACCGAAAGGAGTTAGC | (SEQ ID NO:211) | 54526841 | 55609179 | 41.3 | 0.770 | 0.898 |
| | | | CAGTTTGCTGGGTACTCACTGATAA | (SEQ ID NO:212) | | | | | |
| | | | VIC-ACATGATTGGATAGAGTTA-NFQ | (SEQ ID NO:213) | | | | | |
| | | | FAM-ACATGATTGGTTAGAGTTA-NFQ | (SEQ ID NO:214) | | | | | |
| 240 | rs11587235 | | C_{——}7833748_10 | | 54590171 | 55670818 | 6.1 | 1.000 | 0.180 |
| 241 | rs4926698 | | C_{————}40273_10 | | 54617868 | 55698514 | 49.5 | 0.051 | 0.619 |
| 242 | rs6664825 | | AGTCCCAGTTGAAACTTACTAGATCAGA | (SEQ ID NO:215) | 54728601 | 55809247 | 31.4 | 1.000 | 0.631 |
| | | | CAGCTATTTTACTGTGCACAACCAT | (SEQ ID NO:216) | | | | | |
| | | | VIC-ATAAATGGTCTCTATGGTTCT-NFQ | (SEQ ID NO:217) | | | | | |
| | | | FAM-TGGTCTCTAGGGTTCT-NFQ | (SEQ ID NO:218) | | | | | |
| 243 | rs1412216 | | AGGCAAACAACTTTCTCAGTATCTTCT | (SEQ ID NO:219) | 54855189 | 55935835 | 33.0 | 0.036 | 0.547 |
| | | | ACAGTTGCTTCTCTTTATGAAAATGATCCT | (SEQ ID NO:220) | | | | | |
| | | | VIC-AGCACAAAGAGAGAAA-NFQ | (SEQ ID NO:221) | | | | | |
| | | | FAM-CAGCACAAATAGAGAAA-NFQ | (SEQ ID NO:222) | | | | | |
| 244 | rs778430 | | C_{——}2738616_10 | | 54953165 | 56034137 | 37.3 | 0.762 | 0.440 |
| 245 | rs1557061 | | GGACACTAGAACCTTTGCTACATCT | (SEQ ID NO:223) | 55037128 | 56118100 | 37.8 | 0.538 | 0.311 |
| | | | CTGCTGTTTTTGCTAGTATGCGTAAT | (SEQ ID NO:224) | | | | | |
| | | | VIC-CTGCAATTTATTTTTTG-NFQ | (SEQ ID NO:225) | | | | | |
| | | | FAM-CTGCAATTTATATTTTG-NFQ | (SEQ ID NO:226) | | | | | |
| 246 | rs914833 | | C_11873160_10 | | 55176678 | 56261978 | 18.7 | 1.000 | 0.539 |
| 247 | rs7532239 | | C_11870788_10 | | 55238759 | 56323857 | 30.1 | 1.000 | 0.460 |
| 248 | rs11206831 | PPAP2B | C_{——}1761462_10 | | 55247846 | 56332944 | 23.5 | 0.563 | 0.852 |
| 249 | rs1759752 | PPAP2B | C_{——}1761454_10 | | 55248235 | 56363333 | 45.2 | 0.553 | 0.616 |
| 250 | rs1930760 | PPAP2B | C_{——}1761449_10 | | 55262359 | 56377457 | 34.8 | 0.638 | 0.568 |
| 251 | rs1777284 | PPAP2B | C_{——}8326604_10 | | 55280584 | 56395682 | 43.3 | 0.378 | 0.385 |
| 252 | rs12566304 | PPAP2B | C_11873142_10 | | 55321233 | 56406275 | 34.7 | 0.114 | 0.410 |
| 253 | rs914830 | PPAP2B | C_{——}1761421_20 | | | 56414249 | 48.6 | 0.379 | 0.217 |
| 254 | rs857156 | PRKAA2 | C_{——}9583671_10 | | 55448128 | 56533172 | 49.7 | 1.000 | 0.816 |
| 255 | rs1738403 | AK125198 | C_{——}2821438_10 | | 55531078 | 56616477 | 48.1 | 0.457 | 1.000 |
| 256 | rs652785 | C8A | C_{——}3024292_1_{—} | | 55625247 | 56710645 | 37.5 | 0.640 | 0.420 |
| 257 | rs1411008 | | C_{——}9585012_10 | | 55726421 | 56811543 | 22.0 | 0.311 | 0.851 |
| 258 | rs514412 | DAB1 | C_{———}935471_10 | | 55836403 | 56921487 | 26.1 | 0.849 | 0.864 |
| 259 | rs1504589 | DAB1 | C_{——}3160293_10 | | 55930904 | 57015219 | 43.0 | 0.462 | 0.074 |
| 260 | rs632935 | DAB1 | C_{——}3144357_10 | | 56062978 | 57147300 | 49.7 | 0.655 | 0.806 |
| 261 | rs1556585 | DAB1 | C_{——}1772053_10 | | 56176279 | 57260679 | 39.9 | 0.883 | 0.298 |
| 262 | rs12120223 | DAB1 | C_{——}11287321_10 | | 56259339 | 57343766 | 39.7 | 0.136 | 0.303 |
| 263 | rs7528953 | DAB1 | C_{———}393878_10 | | 56353614 | 57438037 | 17.7 | 0.138 | 0.279 |
| 264 | rs985783 | DAB1 | C_{——}1899963_10 | | 56477154 | 57561719 | 23.7 | 0.680 | 0.575 |
| 265 | rs852778 | DAB1 | C_{——}1900064_10 | | 56580044 | 57664628 | 46.2 | 0.768 | 0.537 |
| 266 | rs1202822 | DAB1 | C_{——}1212518_1_{—} | | 56631211 | 57716125 | 13.3 | 1.000 | 1.000 |
| 267 | rs1188008 | DAB1 | GACCATGAAATACAGAGATGAGTCACA | (SEQ ID NO:227) | 56762803 | 57847717 | 48.7 | 0.896 | 0.222 |
| | | | CCTCTGATTGGTCAGTCCTTCTCA | (SEQ ID NO:228) | | | | | |
| | | | VIC-CTCAGGGAGATTACA-NFQ | (SEQ ID NO:229) | | | | | |
| | | | FAM-TCTCAGGGATATTACA-NFQ | (SEQ ID NO:230) | | | | | |
| 268 | rs4110981 | DAB1 | C_{——}1964002_10 | | 56797091 | 57881967 | 49.8 | 0.033 | 1.000 |
| 269 | rs1213757 | DAB1 | GGATTTCTTCTTGGACTCACACTCT | (SEQ ID NO:231) | 56901236 | 57986150 | 33.9 | 0.258 | 0.894 |
| | | | CCCAACCTGCTCCCACTTTT | (SEQ ID NO:232) | | | | | |
| | | | VIC-CAGTGAATTTGCATTTAG-NFQ | (SEQ ID NO:233) | | | | | |
| | | | FAM-CAGTGAATTTGCGTTTAG-NFQ | (SEQ ID NO:234) | | | | | |
| 270 | rs1416343 | DAB1 | CCTGGAAAATCTAATCGCATGAGGTA | (SEQ ID NO:235) | 56965614 | 58050528 | 16.2 | 0.182 | 1.000 |
| | | | CTGCCCATGCTGAAAATCCTATG | (SEQ ID NO:236) | | | | | |
| | | | VIC-CTGGAAGGAAAACCCCAT-NFQ | (SEQ ID NO:237) | | | | | |
| | | | FAM-TGGAAGGAAAACACCAT-NFQ | (SEQ ID NO:238) | | | | | |
| 271 | rs1341743 | DAB1 | GCATGAGGCACTGAGACTAAGTC | (SEQ ID NO:239) | 57111174 | 58196088 | 9.9 | 0.223 | 0.380 |
| | | | AGTGCAGTGGAAATCAGTCTAAAGG | (SEQ ID NO:240) | | | | | |
| | | | VIC-TGCCGCCTTTTCAT-NFQ | (SEQ ID NO:241) | | | | | |
| | | | FAM-TTGCCCCCTTTTCAT-NFQ | (SEQ ID NO:242) | | | | | |
| 272 | rs338901 | DAB1 | C_{——}3120903_10 | | 57162375 | 58248188 | 40.1 | 0.306 | 0.358 |
| 273 | rs1503646 | DAB1 | C_{———}9586070_10 | | 57252046 | 58337860 | 10.0 | 0.126 | 0.044 |
| 274 | rs232840 | TACSTD2 | C_{———}572140_1_{—} | | 57324571 | 58410636 | 17.7 | 0.311 | 0.288 |
| 275 | rs232795 | AB067502 | C_{——}2968548_10 | | 57416778 | 58503185 | 14.6 | 0.033 | 0.142 |
| 276 | rs11688 | JUN | C_{——}1626096_10 | | 57531826 | 58617910 | 5.1 | 1.000 | 1.000 |
| 277 | rs7552624 | | C_{——}1626068_10 | | 57597277 | 58683353 | 31.5 | 0.513 | 0.875 |
| 278 | rs2764915 | | TCTTTTCAGAGCTCTCCTCAGACT | (SEQ ID NO:243) | 57682591 | 58764375 | 41.1 | 0.769 | 0.178 |
| | | | GACTGGGAAGGAACAGAGAAAGG | (SEQ ID NO:244) | | | | | |
| | | | VIC-ACTCATTGACCTCCTCC-NFQ | (SEQ ID NO:245) | | | | | |
| | | | FAM-CTCATTGAACTCCTCC-NFQ | (SEQ ID NO:246) | | | | | |
| 279 | rs2716140 | | C_{——}1975951_10 | | 57760530 | 58842314 | 38.1 | 0.758 | 0.897 |
| 280 | rs4598514 | | C_{———}290870_10 | | 57807771 | 58889535 | 25.9 | 1.000 | 1.000 |
| 281 | rs6691259 | | C_{——}3124975_10 | | 57898769 | 58980524 | 8.6 | 0.381 | 1.000 |
| 282 | rs331635 | | CTTTCCATTTCCCTCCACTACACT | (SEQ ID NO:247) | 57953675 | 59035459 | 6.0 | 1.000 | 0.376 |
| | | | AACTACATAGAGACTTTCAAGGTGAAGAAG | (SEQ ID NO:248) | | | | | |
| | | | FAM-ACTTGTAAGTCTCCGACCATGCCATG-BHQ1 | (SEQ ID NO:249) | | | | | |
| | | | TET-ACTTGTAAGTCTCTGACCATGCCATGCT-BHQ1 | (SEQ ID NO:250) | | | | | |
| 283 | hcv376342 | FLJ10986 | C_{———}376342_10 | | 58053918 | 59135700 | 6.8 | 1.000 | 0.383 |
| 284 | rs835441 | FLJ10986 | C_{——}9003228_10 | | 58111381 | 59193161 | 25.8 | 0.864 | 0.862 |

TABLE 18
Pairwise Pearson correlation coefficient (r²) for the expression genes identified by
the genomic convergence approach. The lower triangle is for the unaffected group and
upper triangle is for the affected group. Highlighted in bold are the strong LD values.

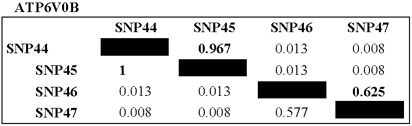

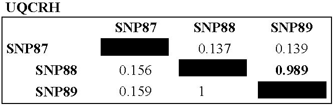

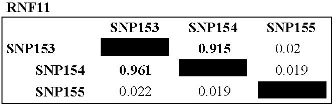

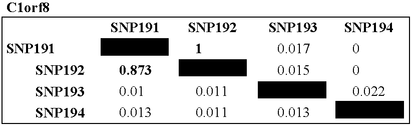

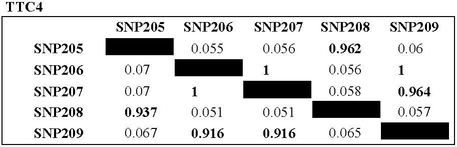

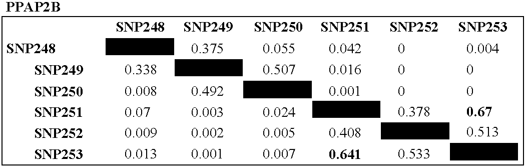

TABLE 19
Characterization of European haplogroups

| Haplogroup | 1719 | 4580 | 7028 | 8251 | 9055 | 10398 | 12308 | 13368 | 13708 | 16391 |
|---|---|---|---|---|---|---|---|---|---|---|
| H | | | C | | | A | | | | |
| I | A | | T | A | | G | | | | A |
| J | | | T | | | G | | | A | |
| K | | | T | | A | G | G | | | |
| T | | | T | | | A | | A | | |
| U | | | T | | | A | G | | | |
| V | | A | T | | | A | | | | |
| W | | | T | A | | A | | | | |
| X | A | | T | | | A | | | | |

TABLE 20
Haplogroup counts and frequencies overall

| | PD cases | | Control | | Total | |
|---|---|---|---|---|---|---|
| | n = 609 | | n = 340 | | n = 949 | |
| Haplogroup | n | Freq. | n | Freq. | n | Freq. |
| H | 273 | 44.8 | 134 | 39.4 | 407 | 42.9 |
| I | 20 | 3.3 | 11 | 3.2 | 31 | 3.3 |
| J | 43 | 7.1 | 38 | 11.2 | 81 | 8.5 |
| K | 34 | 5.6 | 32 | 9.4 | 66 | 6.9 |
| T | 53 | 8.7 | 36 | 10.6 | 89 | 9.4 |
| U | 94 | 15.4 | 41 | 12.1 | 135 | 14.2 |
| V | 24 | 3.9 | 10 | 2.9 | 36 | 3.6 |
| W | 8 | 1.3 | 5 | 1.5 | 13 | 1.4 |
| X | 8 | 1.3 | 5 | 1.5 | 13 | 1.4 |
| other | 52 | 8.5 | 28 | 8.2 | 80 | 8.4 |

TABLE 21
Odds ratio (OR) of mt haplogroups and SNPs overall

| | OR | LB 95% CI | UB 95% CI | p-value |
|---|---|---|---|---|
| Haplogroup | | | | |
| I | 0.83 | 0.38 | 1.83 | 0.65 |
| J | 0.55 | 0.34 | 0.91 | 0.02 |
| K | 0.52 | 0.30 | 0.90 | 0.02 |
| T | 0.74 | 0.46 | 1.21 | 0.23 |
| U | 1.24 | 0.81 | 1.92 | 0.33 |
| V | 1.19 | 0.54 | 2.62 | 0.67 |
| W | 0.67 | 0.20 | 2.11 | 0.48 |
| X | 0.59 | 0.18 | 1.90 | 0.37 |
| other | 0.90 | 0.53 | 1.51 | 0.69 |
| SNP | | | | |
| 1719GA | 1.30 | 0.77 | 2.21 | 0.33 |
| 4580GA | 0.74 | 0.34 | 1.59 | 0.44 |
| 7028TC | 0.83 | 0.63 | 1.09 | 0.18 |
| 8251GA | 1.05 | 0.58 | 1.89 | 0.88 |
| 9055GA | 0.69 | 0.44 | 1.09 | 0.11 |
| 10398GA | 0.53 | 0.39 | 0.73 | 0.0001 |
| 12308AG | 1.04 | 0.75 | 1.45 | 0.80 |
| 13368AG | 1.26 | 0.80 | 1.98 | 0.31 |
| 13708GA | 0.72 | 0.47 | 1.11 | 0.14 |
| 16391AG | 1.06 | 0.49 | 2.29 | 0.88 |

N = 949 total individuals/609 cases; for OR haplogroups were compared to reference haplogroup H

TABLE 22
Association results for mitochondrial haplogroups

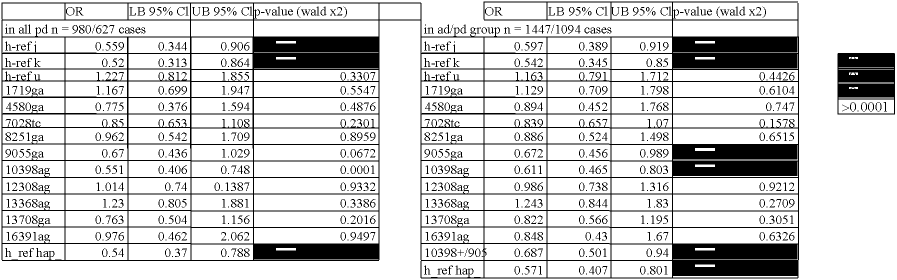

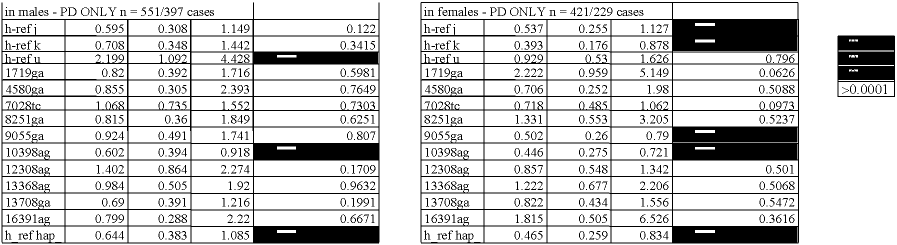

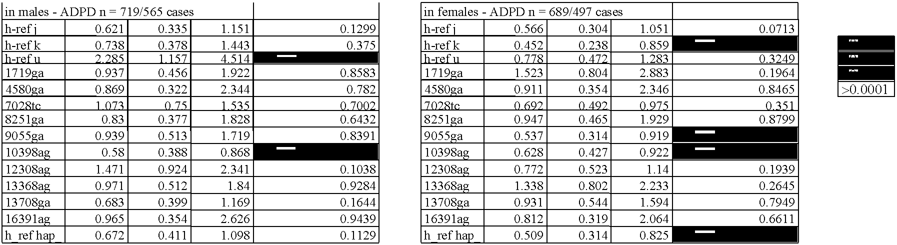

That which is claimed is:

1. A method of identifying a subject as having Parkinson disease and/or having an earlier or later age of developing Parkinson disease and/or having an increased risk of developing Parkinson disease, comprising detecting in the subject the presence of a single nucleotide polymorphism in the eukaryotic translation initiation factor EIF2B3 gene, wherein the single nucleotide polymorphism is correlated with Parkinson disease and/or an earlier or later age of developing Parkinson disease and/or an increased risk of developing Parkinson disease, thereby identifying the subject as having Parkinson disease and/or having an earlier or later age of developing Parkinson disease and/or having an increased risk of developing Parkinson disease.

2. The method of claim 1, wherein the single nucleotide polymorphism in the EIF2B3 gene is selected from the group consisting of rs263977 (SNP 59), rs263978 (SNP 60), rs263965 (SNP 61), rs1022814 (SNP 62), rs12405721 (SNP 63), rs546354 (SNP 64), rs489676 (SNP 67) and any combination of rs263977 (SNP 59), rs263978 (SNP 60), rs263965 (SNP 61), rs10222814 (SNP 62), rs12405721 (SNP 63), rs546354 (SNP 64) and rs489676 (SNP 67).

3. A method of identifying a subject as having Parkinson disease and/or having an increased risk of developing Parkinson disease and/or having an earlier or later age of developing Parkinson disease, comprising detecting in the subject the presence of a haplotype in the EIF2B3 gene of the subject comprising the following single nucleotide polymorphisms:
rs263977_C (SNP 59_C),
rs263978_C (SNP 60_C),
rs546354_G (SNP 64_G),
rs566063_T (SNP 65_T), and
rs364482_G (SNP 66_G).

4. A method of identifying a subject as having Parkinson disease and/or having an increased risk of developing Parkinson disease and/or having an earlier or later age of developing Parkinson disease, comprising detecting in the subject the presence of a haplotype in the EIF2B3 gene of the subject comprising the following single nucleotide polymorphisms:
rs263977_A (SNP 59_A),
rs263978_C (SNP 60_C),
rs546354_A (SNP 64_A),
rs566063_T (SNP 65_T), and
rs364482_G (SNP 66_G).
